(19) United States
(12) Patent Application Publication
(10) Pub. No.: US 2006/0147520 A1
(43) Pub. Date: Jul. 6, 2006

(54) Treatment of pulmonary hypertension by inhaled iloprost with a microparticle formulation

(76) Inventor: Curtis Ruegg, Redwood City, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/189,553

(22) Filed: Jul. 26, 2005

Related U.S. Application Data
(60) Provisional application No. 60/591,253, filed on Jul. 26, 2004.

(51) Int. Cl. A61K 31/7024; A61K 31/557; A61K 9/20; A61K 9/14

(52) U.S. Cl. 424/464; 424/489; 514/53; 514/61

(57) ABSTRACT

Microparticles comprising iloprost are disclosed. In some embodiments, the microparticles are used to treat pulmonary hypertension. Devices comprising the microparticles are also disclosed. Combination therapies utilizing the microparticles are also provided.

### RELATED APPLICATIONS

The present application is a nonprovisional of U.S. Provisional Patent Application Ser. No. 60/591,253, entitled Treatment of Pulmonary Hypertension by Inhaled Iloprost with a Microparticle Formulation filed Jul. 26, 2004, the disclosure of which is incorporated by reference herein in its entirety.

### FIELD OF THE INVENTION

Preferred embodiments of the present invention are related to microparticles comprising iloprost and therapeutic methods for treating pulmonary hypertension by pulmonary delivery of such microparticles.

### BACKGROUND OF THE INVENTION

Pulmonary hypertension is a debilitating disease characterized by an increase in pulmonary vascular resistance leading to right ventricular failure and death. Pulmonary hypertension (PH) with no apparent cause is termed primary pulmonary hypertension (PPH). Pulmonary hypertension includes pulmonary arterial hypertension as well as other disorders. Recently, various pathophysiological changes associated with this disorder, including vasoconstriction, vascular remodeling (i.e. proliferation of both media and intima of the pulmonary resistance vessels), and in situ thrombosis have been characterized (e.g., D'Alonzo, G. E. et al. 1991 Ann Intern Med 115:343-349; Palevsky, H. I. et al. 1989 Circulation 80:1207-1221; Rubin, L. J. 1997 N Engl J Med 336:111-117; Wagenvoort, C. A. & Wagenvoort, N. 1970 Circulation 42:1163-1184; Wood, P. 1958 Br Heart J 20:557-570). Impairment of vascular and endothelial homeostasis is evidenced from a reduced synthesis of prostacyclin (PGI₂), increased thromboxane production, decreased formation of nitric oxide and increased synthesis of endothelin-1 (Giaid, A. & Saleh, D. 1995 N Engl J Med 333:214-221; Xue, C & Johns, R. A. 1995 N Engl J Med 333:1642-1644). The intracellular free calcium concentration of vascular smooth muscle cells of pulmonary arteries in PPH has been reported to be elevated.

Current therapies for pulmonary hypertension utilize calcium channel antagonists, prostacyclins, endothelin receptor antagonists and long-term anticoagulant therapy. However, each treatment has limitations and side effects.

The present invention includes microparticles comprising iloprost which are convenient to administer, thereby leading to enhanced patient compliance. In some embodiments, the microparticles may be administered in a single puff. In other embodiments, the microparticles may be provided in a sustained-release inhalable formulation, which exhibits fewer or no adverse effects (i.e., less toxicity) and a favorable profile in terms effectiveness in patients in different stages of PH.

### SUMMARY OF THE INVENTION

Some embodiments of the present invention are described in the following paragraphs.

A first embodiment of the present invention relates to microparticles comprising iloprost therein. In some aspects of the first embodiment, the microparticles provide a dosage of iloprost which provides an efficacious amount of iloprost when the microparticles are administered 1 to 10 times daily. In other aspects of the first embodiment, the microparticles provide a dosage of iloprost which provides an efficacious amount of iloprost when the microparticles are administered 1 to 4 times daily. In additional aspects of the first embodiment, the microparticles provide a dosage of iloprost which provides an efficacious amount of iloprost when the microparticles are administered 3 to 4 times daily. In some aspects of the first embodiment, the microparticles are in the form of a dry powder. In other aspects of the first embodiment, the microparticles have a porosity which facilitates the continued release of an efficacious amount of iloprost more than 2 hours after the microparticles have been inhaled by a human subject. In further aspects of the first embodiment, more than about 50% by weight of the iloprost is released from the microparticles by 24 hours after inhalation by a human subject. In additional aspects of the first embodiment, the microparticles are porous. For example, in some aspects of the first embodiment, the microparticles release an effective amount of iloprost over a duration of at least two hours from inhalation of the microparticles by a human subject. In some aspects of the first embodiment, substantially all of the iloprost is released by 24 hours from inhalation of the microparticles by a human subject. In still further aspects of the first embodiment, the microparticles have a volume average diameter of between about 0.1 and about micrometers. In some aspects of the first embodiment, the microparticles have an average porosity between about 15% and about 90%. In other aspects of the first embodiment, the microparticles comprise a matrix material which reduces the rate at which the iloprost is released from the microparticles. For example, the matrix material may be selected from the group consisting of a polymer, a lipid, a salt, a hydrophobic small molecule or a combination of any of the foregoing. The matrix material may comprise at least about 5% by weight of the microparticle in some aspects of the first embodiment. In other aspects of the first embodiment, the microparticles comprise a surfactant. In further aspects of the first embodiment, the surfactant is present in amount less than about 10% by weight of the microparticles. In some aspects of the first embodiment, the iloprost is present in an amount from about 1% to about 70% by weight of the microparticles. In other aspects of the first embodiment, iloprost is present in an amount from about 0.1% to 20% by weight of the microparticles. In further aspects of the first embodiment, the microparticles comprise a matrix having one or more lipids, hydrophobic compounds, or amphiphilic compounds incorporated in the matrix. In additional aspects of the first embodiment, the microparticles comprise components which limit diffusion of the drug out of the microparticle. In still further aspects of the first embodiment, the microparticles comprise components for modifying the degradation kinetics of the microparticles. In other aspects of the first embodiment, the microparticles have a tap density of less than 0.4 gm/cm³. In some aspects of the first embodiment, the microparticles comprise an amino acid, a salt of an amino acid, or an amino acid analog.

A second embodiment of the present invention relates to a method of reducing the symptoms of pulmonary hypertension comprising administering microparticles according to the first embodiment or any aspect thereof to a subject suffering from pulmonary hypertension.

A third embodiment of the present invention relates to a therapeutic combination for the treatment of PH, comprising microparticles according to the first embodiment or any aspect thereof and at least one additional pharmaceutical agent selected from the group consisting of an endothelin receptor antagonist, a PDE inhibitor, and a calcium channel blocker, wherein the iloprost and the at least one additional agent are provided at dosages sufficient to ameliorate at least one symptom associated with PH. In some aspects of the third embodiment, the endothelin receptor antagonist is selected from the group consisting of bosentan, sitaxentan, and ambrisentan. In other aspects of the third embodiment, the at least one additional agent is bosentan. In some aspects of the third embodiment, the at least one additional agent comprises a PDE inhibitor selected from the group consisting of sildenafil (Viagra®), tadalafil (Cialis®) and vardenafil (LEVITRA®). In other aspects of the third embodiment, the microparticles of the first embodiment or any aspect thereof further comprise the at least one additional pharmaceutical agent. In additional aspects of the third embodiment, the at least one additional pharmaceutical agent is provided in microparticles distinct from said microparticles of the first embodiment or any aspect thereof. In further aspects of the first embodiment, the at least one additional pharmaceutical agent is provided in a form other than microparticles.

A fourth embodiment of the present invention relates to a method of treating PH, comprising administering effective amounts of microparticles according to the first embodiment or any aspect thereof and administering at least one additional agent, selected from the group consisting of an endothelin receptor antagonist, a PDE inhibitor, and a calcium channel blocker. In some aspects of the fourth embodiment, the endothelin receptor antagonist is selected from the group consisting of bosentan, sitaxentan, and ambrisentan. In other aspects of the fourth embodiment, the at least one additional agent is bosentan. In some aspects of the fourth embodiment, the at least one additional agent comprises a PDE inhibitor selected from the group consisting of sildenafil (Viagra®), tadalafil (Cialis®) and vardenafil (LEVITRA®). In additional aspects of the fourth embodiment, the microparticles of the first embodiment or any aspect thereof further comprise the at least one additional pharmaceutical agent. In still further aspects of the fourth embodiment, the at least one additional pharmaceutical agent is administered in microparticles distinct from the microparticles of the first embodiment or any aspect thereof. In additional aspects of the fourth embodiment, the at least one additional pharmaceutical agent is administered in a form other than microparticles.

A fifth embodiment of the present invention relates to an inhalation device comprising any one of the compositions of the first embodiment or any aspect thereof. In some aspects of the fifth embodiment, the device is selected from the group consisting of a dry powder inhalation device and a metered dose inhaler.

A sixth embodiment of the present invention relates to a composition comprising a solid dose delivery system comprising a vehicle and an effective amount of iloprost wherein the vehicle comprises a hydrophobic derivatized carbohydrate (HDC). In one aspect of the sixth embodiment, the composition further comprises at least one physiologically acceptable glass selected from the group consisting of carboxylate, nitrate, sulfate, and bisulfate. In another aspect of the sixth embodiment, the HDC has a carbohydrate backbone and more than one hydroxyl group substituted with a less hydrophilic derivative thereof. For example, the derivative may be an ester or ether of any carbon chain length or type or any functional modifications thereof, wherein the functional modifications are selected from the group consisting of replacing the oxygen atom by a heteroatom. In some aspects of the sixth embodiment, the HDC is selected from the group consisting of 6:6′-bis(β-Tetraacetyl glucuronyl)hexaacetyl trehalose, sorbitol hexaacetate, α-Glucose pentaacetate, β-Glucose pentaacetate, 1-0-Octyl-β-D-Glucose tetraacetate, trehalose octaacetate, trehalose octapropanoate, sucrose octaacetate, sucrose octapropanoate, cellobiose octaacetate, cellobiose octapropanoate, raffinose undecaacetate and raffinose undecapropanoate. In other aspects of the sixth embodiment, the guest substance has increased stability in the presence of elevated temperatures or organic solvents. In further aspects of the sixth embodiment, the form of the solid dose is selected from the group consisting of microparticles, microspheres and powders. In another aspect of the sixth embodiment, the composition further comprises a pharmaceutical agent in addition to iloprost, wherein said pharmaceutical agent in addition to iloprost is selected from the group consisting of vasodilators, antihypertensive agents, cardiovascular drugs, an endothelin receptor antagonist, a PDE inhibitor, and a calcium channel blocker, wherein the iloprost and the at least one additional agent are provided at dosages sufficient to ameliorate at least one symptom associated with PH. In one aspect of the sixth embodiment, the vehicle comprises a hydrophobic derivatized carbohydrate (HDC) in which the iloprost can be dried and stored. In another aspect of the sixth embodiment, the vehicle comprises a hydrophobic derivatized carbohydrate (HDC) in which the iloprost can be dried and stored without losses in activity.

In an additional aspect of the sixth embodiment, the hydrophobic derivatized carbohydrate (HDC) is non-toxic. In one aspect of the sixth embodiment, the vehicle comprises a hydrophobic derivatized carbohydrate (HDC) which is glassy or amorphous. In another aspect of the sixth embodiment, the composition is capable of controlled release of the iloprost. In a further aspect of the sixth embodiment, the composition is resistant to devitrification. In one aspect of the sixth embodiment, the HDC is a carbohydrate no greater than a pentasaccharide, and wherein more than one hydroxyl group of the HDC is derivatized as an ester or ether. In one aspect of the sixth embodiment, the composition further comprises a surfactant. For example, the surfactant may have a hydrophile-lipophile balance of at least about 3. In some instances, the surfactant is selected from the group consisting of dipalmitoyl phosphatidylglycerol, dipalmitoyl phosphatidylcholine, glyceryl monostearate, sorbitan monolaurate, polyoxyethylene-4-lauryl ether, polyethylene glycol 400 monostearate, polyoxyethylene-4-sorbitan monolaurate, polyoxyethylene-20-sorbitan monopalmitate, polyoxyethylene-40-stearate, sodium oleate and sodium lauryl sulfate and lung surfactants. In some instances, the composition provides increased bioavailability of the iloprost. In other instances, the composition is obtained by dissolving or suspending the iloprost, the surface active agent and the hydrophobically derivatized carbohydrate in at least one solvent therefor and evaporating the solvent from the mixture. In some instances, the evaporating is by spray drying. In other instances, the composition further comprises a pharmaceutical agent in addition to iloprost, wherein said pharmaceutical agent in addition to iloprost is selected from the group consisting of vasodilators, antihypertensive agents, cardiovascular drugs, an endothelin receptor antagonist, a PDE inhibitor, and a calcium channel blocker, wherein the iloprost and the at least one additional agent are provided at dosages sufficient to ameliorate at least one symptom associated with PH. In some instances, the hydrophobically derivatized carbohydrate is selected from the group consisting of sorbitol hexaacetate (SHAC), α-glucose pentaacetate (α-GPAC), β-glucose pentaacetate (β-GPAC), 1-0-Octyl-β-D-glucose tetraacetate (OGTA), trehalose octaacetate (TOAC), trehalose octapropionate (TOP), trehalose octa-3,3,dimethylbutyrate (T033DMB), trehalose diisobutyrate hexaacetate, trehalose octaisobutyrate, lactose octaacetate, sucrose octaacetate (SOAC), cellobiose octaacetate (COAC), raffinose undecaacetate (RUDA), sucrose octapropanoate, cellobiose octapropanoate, raffinose undecapropanoate, tetra-0-methyl trehalose, trehalose octapivalate, trehalose hexaacetate dipivalate and di-0-methyl-hexa-0-actyl sucrose and mixtures thereof. For example, the hydrophobically derivatized carbohydrate may be a trehalose derivative and comprises: 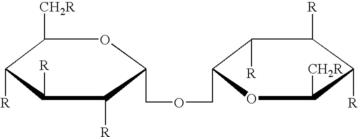
where R represents a hydroxyl group, or less hydrophilic derivative thereof, including an ester or ether or any functional modifications thereof where at least one R is not hydroxyl but a hydrophobic derivative; where functional modifications include where the oxygen atom is replaced by a heteroatom, such as N or S and where R can be of any chain length from C₂ upwards and can be straight, branched, cyclic or modified and mixtures thereof. In some instances, the pharmaceutical composition is in powder form, suspended in an aqueous solution.

In another aspect of the sixth embodiment, the composition further comprises a stabilizing polyol. In some instances the stabilizing polyol is selected from the group consisting of monosaccharides, disaccharides, trisaccharides, oligosaccharides and their corresponding sugar alcohols, polysaccharides and chemically modified carbohydrates such as hydroxyethyl starch and sugar copolymers and Ficoll. In some instances, the stabilizing polyol is trehalose. In one aspect of the sixth embodiment wherein the composition comprises a polyol, the composition further comprises at least one physiologically acceptable glass selected from the group consisting of carboxylate, nitrate, sulfate, and bisulfate. In another aspect of the sixth embodiment wherein the composition comprises a polyol, the HDC has a carbohydrate backbone and more than one hydroxyl group substituted with a less hydrophilic derivative thereof. In a further aspect of the sixth embodiment wherein the composition comprises a polyol, the HDC is selected from the group consisting of 6:6′-bis(β-Tetraacetyl glucuronyl)hexaacetyl trehalose, sorbitol hexaacetate, α-Glucose pentaacetate, β-Glucose pentaacetate, 1-0-Octyl-β-D-Glucose tetraacetate, trehalose octaacetate, trehalose octapropanoate, sucrose octaacetate, sucrose octapropanoate, cellobiose octaacetate, cellobiose octapropanoate, raffinose undecaacetate and raffinose undecapropanoate. In another aspect of the sixth embodiment wherein the composition comprises a polyol, the iloprost has increased stability in the presence of elevated temperatures or organic solvents. In yet another aspect of the sixth embodiment wherein the composition comprises a polyol, the form of the solid dose is selected from the group consisting of microparticles, microspheres, and powders.

A seventh embodiment of the present invention relates to a composition comprising iloprost and a modified glycoside, said modified glycoside having the formula:
(Y)ₙ—X
wherein Y represents a saccharide subunit, n is 1-6, and, when n is greater than 1, the subunits are linked in a linear or branch chain by glycosidic linkages and wherein X is a 5 or 6 carbon monosaccharide polyalcohol, and wherein the polyalcohol has a hydroxy group linked via a glycosidic bond to the anomeric carbon of one of the saccharide subunits, and wherein the glycoside has at least one hydroxy group derivatized in the form of an ester, mixed ester, ether or mixed ether, and wherein the modified glycoside is in the form of a vitreous glass matrix and has a bioactive substance incorporated therein. In one aspect of the seventh embodiment, the saccharide subunits, Y, are the same or different and are selected from the group consisting of glucose, galactose, fructose, ribulose, mannose, ribose, arabinose, xylose, lyxose, allose, altrose, and gulose. In another aspect of the seventh embodiment, the polyalcohol is selected from the group consisting of erythritol, ribitol, xylitol, galactitol, glucitol and mannitol. In a further aspect of the seventh embodiment, the modified glycoside is a hydrogenated maltooligosaccharide or isomaltooligosaccharide. For example, the hydrogenated maltooligosaccharide may be selected from the group consisting of maltotritol, maltotetraitol, maltopentaitol, maltohexaitol, maltooctaitol, maltononaitol and maltodecaitol. In one aspect of the seventh embodiment, the modified glycoside is selected from the group consisting of hydrophobic esters, mixed esters, ethers or mixed ethers of a glycoside of a sugar alcohol. In another aspect of the seventh embodiment, the modified glycoside is selected from the group consisting of lactitol nonaacetate, palatinit nonaacetate, glycopyranosyl sorbitol nonaacetate, glucopyranosyl mannitol nonaacetate, maltitol nonaacetate and mixtures thereof. In a further aspect of the seventh embodiment, the composition further comprises at least one physiologically acceptable glass selected from the group consisting of carboxylate, nitrate, sulfate, bisulfate, a hydrophobic carbohydrate derivative, and combinations thereof. In one aspect of the seventh embodiment, the composition is in the form of a solid delivery system selected from the group consisting of microparticles, microspheres, and powders. In another aspect of the seventh embodiment, the composition further comprises a surfactant. For example, the surfactant may have a hydrophile-lipophile balance of at least about 3. In some instances, the surfactant is selected from the group consisting of dipalmitoyl phosphatidylglycerol, dipalmitoyl phosphatidylcholine, glyceryl monostearate, sorbitan monolaurate, polyoxyethylene-4-lauryl ether, polyethylene glycol 400 monostearate, polyoxyethylene-4-sorbitan monolaurate, polyoxyethylene-20-sorbitan monopalmitate, polyoxyethylene-40-stearate, sodium oleate and sodium lauryl sulfate and lung surfactants. In one aspect of the seventh embodiment, the composition further comprises a stabilizing polyol. For example, the stabilizing polyol may be selected from the group consisting of monosaccharides, disaccharides, trisaccharides, oligosaccharides and their corresponding sugar alcohols, polysaccharides and chemically modified carbohydrates such as hydroxyethyl starch and sugar copolymers and Ficoll. In some instances, the stabilizing polyol is trehalose. In one aspect of the seventh embodiment, the composition further comprises a pharmaceutical agent in addition to iloprost, wherein said pharmaceutical agent in addition to iloprost is selected from the group consisting of vasodilators, antihypertensive agents, cardiovascular drugs, an endothelin receptor antagonist, a PDE inhibitor, and a calcium channel blocker, wherein the iloprost and the at least one additional agent are provided at dosages sufficient to ameliorate at least one symptom associated with PH.

An eighth embodiment of the present invention is a pharmaceutical composition for pulmonary delivery comprising an intimate mixture of a therapeutically effective amount of iloprost, a surface active agent, and a hydrophobically derivatized carbohydrate (HDC) where the composition is in powder form. In one aspect of the eighth embodiment, the composition provides increased bioavailability of the iloprost to the pulmonary system. In another aspect of the eighth embodiment, the surface active agent forms a continuous phase with the HDC. In yet another aspect of the eighth embodiment, the surface active agent is a surfactant with a hydrophile-lipophile balance. In some instances, the hydrophile-lipophile balance is of at least about 3. In one aspect of the eighth embodiment, the surfactant is selected from the group consisting of dipalmitoyl phosphatidylglycerol, dipalmitoyl phosphatidylcholine, glyceryl monostearate, sorbitan monolaurate, polyoxyethylene-4-lauryl ether, polyethylene glycol 400 monostearate, polyoxyethylene-4-sorbitan monolaurate, polyoxyethylene-20-sorbitan monopalmitate, polyoxyethylene-40-stearate, sodium oleate sodium lauryl sulfate and lung surfactants. In another aspect of the eighth embodiment, mucosal delivery is via by-inhalation delivery. In a further aspect of the eighth embodiment, the powder contains particles with a mass median aerodynamic diameter of about 0.1 to 10 microns. In one aspect of the eighth embodiment, the powder contains particles with a mass median aerodynamic diameter of about 0.5 to 5 microns. In one aspect of the eighth embodiment, the powder contains particles with a mass median aerodynamic diameter of about 1 to 4 microns. In a further aspect of the eighth embodiment, the intimate mixture is obtained by dissolving or suspending the bioactive agent and the hydrophobically derivatized carbohydrate in at least one solvent therefor and evaporating the solvent from the mixture. In one aspect of the eighth embodiment, the evaporating is by spray drying. In another aspect of the eighth embodiment, the composition further comprises a pharmaceutical agent in addition to iloprost, wherein said pharmaceutical agent in addition to iloprost is selected from the group consisting of vasodilators, antihypertensive agents, cardiovascular drugs, an endothelin receptor antagonist, a PDE inhibitor, and a calcium channel blocker, wherein the iloprost and the at least one additional agent are provided at dosages sufficient to ameliorate at least one symptom associated with PH. In one aspect of the eighth embodiment, the hydrophobically derivatized carbohydrate is selected from the group consisting of sorbitol hexaacetate (SHAC), α-glucose pentaacetate (α-GPAC), β-glucose pentaacetate (β-GPAC), 1-0-Octyl-β-D-glucose tetraacetate (OGTA), trehalose octaacetate (TOAQ, tetralose octapropionate (TOP), trehalose octa-3,3,dimethylbutyrate (T033DMB), trehalose diisobutyrate hexaacetate, trehalose octaisobutyrate, lactose octaacetate, sucrose octaacetate (SOAC), cellobiose octaacetate (COAC), raffinose undecaacetate (RUDA), sucrose octapropanoate, cellobiose octapropanoate, raffinose undecapropanoate, tetra-0-methyl trehalose, trehalose octapivalate, trehalose hexaacetate dipivalate and di-0-methyl-hexa-0-actyl sucrose and mixtures thereof. In another aspect of the eighth embodiment, the hydrophobically derivatized carbohydrate is a trehalose derivative and comprises 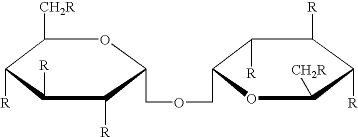
where R represents a hydroxyl group, or less hydrophilic derivative thereof, including an ester or ether or any functional modifications thereof where at least one R is not hydroxyl but a hydrophobic derivative; where functional modifications include where the oxygen atom is replaced by a heteroatom, such as N or S and where R can be of any chain length from C₂ upwards and can be straight, branched, cyclic or modified and mixtures thereof. In one aspect of the eighth embodiment, the composition is suspended in an aqueous solution. In another aspect of the eighth embodiment, the powder contains particles with a mass median aerodynamic diameter of 1.5-3 microns. In a further aspect of the eighth embodiment, the HDC is 6:6′-bis(β-Tetraacetyl glucuronyl)hexaacetyl trehalose.

A ninth embodiment of the present invention relates to a composition comprising iloprost and 6:6′-bis(β-Tetraacetyl glucuronyl)hexaacetyl trehalose. In one aspect of the ninth embodiment, the composition further comprises a surfactant. In another aspect of the ninth embodiment, the composition further comprises trehalose. In a further aspect of the ninth embodiment, the iloprost is present at a concentration from about 0.01%-about 30% by weight. In another aspect of the ninth embodiment, the iloprost is present at a concentration from about 0.05%-about 20% by weight. In one aspect of the ninth embodiment, the iloprost is present at a concentration from about 0.1%-about 5% by weight. In some instances, the surfactant is selected from the group consisting of dipalmitoyl phosphatidylglycerol and dipalmitoyl phosphatidylcholine. In other instances, the surfactant is present in a concentration of about 0.01%-about 30% by weight. In further instances, surfactant is present in a concentration of 0.1%-20% by weight. In some instances, the surfactant is present in a concentration of about 0.1%-about 10% by weight. For example, the surfact may be present in a concentration of about 0.1%-5% by weight.

A tenth embodiment of the present invention is microparticles comprising iloprost therein. In one aspect of the tenth embodiment, the microparticles provide a dosage of iloprost which provides an efficacious amount of iloprost when said microparticles are administered 1 to 10 times daily. In another aspect of the tenth embodiment, the microparticles provide a dosage of iloprost which provides an efficacious amount of iloprost when said microparticles are administered 1 to 4 times daily. In a further aspect of the tenth embodiment, the microparticles provide a dosage of iloprost which provides an efficacious amount of iloprost when said microparticles are administered 3 to 4 times daily.

In one aspect of the tenth embodiment, the microparticles are in the form of a dry powder. In another aspect of the tenth embodiment, the microparticles release an effective amount of iloprost over a duration of at least two hours from inhalation of said microparticles by a human subject. In a further aspect of the tenth embodiment, substantially all of the iloprost is released by 24 hours from inhalation of said microparticles by a human subject. In one aspect of the tenth embodiment, the microparticles further comprise a carbohydrate or derivative of a carbohydrate. In another aspect of the tenth embodiment, the derivative of a carbohydrate is an ether or ester. In a further aspect of the tenth embodiment, the derivative of a carbohydrate is an ester.

An eleventh embodiment of the present invention relates to a method of treating PH, comprising administering effective amounts of microparticles comprising iloprost to an individual suffering from PH. The microparticles may be any of the microparticles described in the present application.

A twelfth embodiment of the present invention relates to an inhalation device comprising microparticles comprising iloprost. The microparticles may be any of the microparticles described in the present application. In some aspects of the twelfth embodiment, the device is selected from the group consisting of a dry powder inhalation device and a metered dose inhaler.

Other embodiments of the present invention are described throughout the present application.

### BRIEF DESCRIPTION OF THE DRAWINGS

FIG. 1A shows the release profile of the RDD/05/233, RDD/05/255 and RDD/05/257 formulations over a time period of 300 minutes.

FIG. 1B shows the release profile of the RDD/05/233, RDD/05/255 and RDD/05/257 formulations over a time period of 300 minutes.

FIG. 2 shows the release profile of the RDD/05/233, RDD/05/255 and RDD/05/257 formulations over a time period of 300 minutes.

FIG. 3 shows the release profile of the RDD/05/233 and RDD/05/262 formulations over a time period of 300 minutes.

FIG. 4 shows the release profile of the RDD/05/233 and RDD/05/267 formulations over a time period of 300 minutes.

FIG. 5 shows the release profile of the RDD/05/233 and RDD/05/270 formulations over a time period of 300 minutes.

FIG. 6 shows the release profile of the RDD/05/273 and RDD/05/274 formulations over a time period of 300 minutes.

### DETAILED DESCRIPTION OF THE PREFERRED EMBODIMENT

Preferred embodiments of the present invention relate to microparticles comprising iloprost. In some embodiments, the microparticles comprising iloprost are administered with another pharmaceutical agent. In such embodiments, the other pharmaceutical agent may be in the same microparticles as the iloprost, in different microparticles than the iloprost or it may not be in the form of a microparticle.

Microparticle Formulations of Iloprost for Pulmonary Delivery

Some embodiments of the present invention relate to a composition comprising microparticles containing iloprost and/or another pharmaceutical agent to be administered in addition to iloprost therein. Microparticle compositions have been described in U.S. Patent Application Publication No. 2003/0068277A1 to Vanbever, et al., U.S. Pat. No. 6,060,069 to Hill et al., PCT WO 01/13891 to Basu et al., U.S. Patent Application No. 2004/0105821, U.S. Pat. No. 6,586,008, and U.S. Pat. No. 6,730,322, U.S. Pat. No. 6,586,006, U.S. Pat. No. 6,517,860, U.S. Pat. No. 6,352,722, and U.S. patent application Ser. No. 09/923,023 (published as US 2002/0009464) the disclosures of which are incorporated herein by reference in their entireties. Iloprost (see U.S. Pat. No. 4,692,464; incorporated herein in its entirety by reference thereto) is a stable analogue of prostacyclin that is associated with a longer duration of vasodilatation (Fitscha P. et al. 1987 Adv Prostaglandin Thromboxane Leukot Res 17:450-454). When administered by aerosolization to patients with pulmonary hypertension, its pulmonary vasodilative potency was similar to that of prostacyclin, but its effects lasted for 30 to 90 minutes, as compared with only 15 minutes for the prostacyclin (Hoeper M. M. et al. 2000 J Am Coll Cardiol 35:176-182; Olschewski H. et al. 1999 Am J Respir Crit Care Med 160:600-607; Olschewski H. et al. 1996 Ann Intern Med 124:820-824; Gessler T. et al. 2001 Eur Respir J 17:14-19; Wensel R. et al. 2000 Circulation 101:2388-2392). Several open-label, uncontrolled studies of patients with severe pulmonary hypertension suggested that long-term use of aerosolized iloprost results in substantial clinical improvement (Olschewski H. et al. 1999 Am J Respir Crit Care Med 160:600-607; Olschewski H. et al. 1996 Ann Intern Med 124:820-824; Hoeper M. M. et al. 2000 N Engl J Med 342:1866-1870; Olschewski H. et al. 1998 Intensive Care Med 24:631-634.; Stricker H. et al. 1999 Schweiz Med Wochenschr 129:923-927; Olschewski H. et al. 2000 Ann Intern Med 132:435-443; Beghetti M. et al. 2001 Heart 86:E10-E10). A multi-center randomized placebo controlled study of patients with severe PAH has demonstrated improved exercise capacity in patients receiving iloprost versus those receiving placebo (Olschewski H et al 2002 NEJM 2002;345:322-9).

Microparticles are convenient to administer, thereby enhancing the extent of patient compliance. In some embodiments, the microparticles may be administered in a single puff. In other embodiments, the microparticles are formulated to provide sustained release of iloprost. The microparticles may facilitate local delivery of iloprost to the lungs or systemic delivery via the lungs. In some embodiments, the microparticles enable less frequent dosing of iloprost. For example, in some embodiments, the microparticles provide efficacious 1-10 times daily dosing of iloprost useful in the treatment of PH. In other embodiments, the microparticles permit 1-4 times or 3-4 times daily dosing of iloprost.

Aerosols for the delivery of therapeutic agents to the respiratory tract have been described, for example, Adjei, A. and Garren, J. Pharm. Res., 7: 565-569 (1990); and Zanen, P. and Lamm, J.-W. J. Int. J. Pharm., 114:111-115 (1995). The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung. Gonda, I. “Aerosols for delivery of therapeutic and diagnostic agents to the respiratory tract,” in Critical Reviews in Therapeutic Drug Carrier Systems, 6:273-313 (1990). The deep lung, or alveoli, is the primary target of inhaled therapeutic aerosols for systemic drug delivery.

Considerable attention has been devoted to the design of therapeutic aerosol inhalers to improve the efficiency of inhalation therapies. Timsina et. al., Int. J. Pharm., 101: 1-13 (1995); and Tansey, I. P., Spray Technol. Market, 4: 26-29 (1994). Attention has also been given to the design of dry powder aerosol surface texture, regarding particularly the need to avoid particle aggregation, a phenomenon which considerably diminishes the efficiency of inhalation therapies. French, D. L., Edwards, D. A. and Niven, R. W., J. Aerosol Sci., 27: 769-783 (1996). Dry powder formulations (“DPFs”) with large particle size have improved flowability characteristics, such as less aggregation (Visser, J., Powder Technology 58: 1-10 (1989)), easier aerosolization, and potentially less phagocytosis. Rudt, S. and R. H. Muller, J. Controlled Release, 22: 263-272 (1992); Tabata, Y. and Y. Ikada, J. Biomed. Mater. Res., 22: 837-858 (1988). Dry powder aerosols for inhalation therapy are generally produced with mean geometric diameters primarily in the range of less than 5 micrometers. Ganderton, D., J Biopharmaceutical Sciences, 3: 101-105 (1992); and Gonda, I. “Physico-Chemical Principles in Aerosol Delivery,” in Topics in Pharmaceutical Sciences 1991, Crommelin, D. J. and K. K. Midha, Eds., Medpharm Scientific Publishers, Stuttgart, pp. 95-115, 1992. Large “carrier” particles (containing no drug) have been co-delivered with therapeutic aerosols to aid in achieving efficient aerosolization among other possible benefits. French, D. L., Edwards, D. A. and Niven, R. W., J. Aerosol Sci., 27: 769-783 (1996).

The human lungs can remove or rapidly degrade hydrolytically cleavable deposited aerosols over periods ranging from minutes to hours. In the upper airways, ciliated epithelia contribute to the “mucociliary escalator” by which particles are swept from the airways toward the mouth. Pavia, D. “Lung Mucociliary Clearance,” in Aerosols and the Lung. Clinical and Experimental Aspects, Clarke, S. W. and Pavia, D., Eds., Butterworths, London, 1984. Anderson, Am. Rev. Respir. Dis., 140: 1317-1324 (1989). In the deep lungs, alveolar macrophages are capable of phagocytosing particles soon after their deposition. Warheit, M. B. and Hartsky, M. A., Microscopy Res. Tech., 26: 412-422 (1993); Brain, J. D., “Physiology and Pathophysiology of Pulmonary Macrophages,” in The Reticuloendothelial System, S. M. Reichard and J. Filkins, Eds., Plenum, N.Y., pp. 315-327, 1985; Dorries, A. M. and Valberg, P. A., Am. Rev. Resp. Disease 146: 831-837 (1991); and Gehr, P., Microscopy Res. and Tech., 26: 423-436 (1993). As the diameter of particles exceeds 3 micrometers, there is increasingly less phagocytosis by macrophages. Kawaguchi, H., Biomaterials 7: 61-66 (1986); Krenis, L. J. and Strauss, B., Proc. Soc. Exp. Med., 107: 748-750 (1961); and Rudt, S. and Muller, R. H., J. Contr. Rel., 22: 263-272 (1992). However, increasing the particle size also has been found to minimize the probability of particles (possessing standard mass density) entering the airways and acini due to excessive deposition in the oropharyngeal or nasal regions. Heyder, J., J. Aerosol Sci., 17: 811-825 (1986).

Local and systemic inhalation therapies can often benefit from a relatively slow controlled release of the therapeutic agent. Gonda, I., “Physico-chemical principles in aerosol delivery,” in: Topics in Pharmaceutical Sciences 1991, D. J. A. Crommelin and K. K. Midha, Eds., Stuttgart: Medpharm Scientific Publishers, pp. 95-117 (1992). Slow release from a therapeutic aerosol can prolong the residence of an administered drug in the airways or acini, and diminish the rate of drug appearance in the bloodstream. Also, patient compliance is increased by reducing the frequency of dosing. Langer, R., Science, 249: 1527-1533 (1990); and Gonda, I., “Aerosols for delivery of therapeutic and diagnostic agents to the respiratory tract,” in Critical Reviews in Therapeutic Drug Carrier Systems 6: 273-313 (1990).

Controlled release drug delivery to the lung may simplify the way in which many drugs are taken. Gonda, I., Adv. Drug Del. Rev., 5: 1-9 (1990); and Zeng, X., et al., Int. J. Pharm., 124: 149-164 (1995). Pulmonary drug delivery is an attractive alternative to oral, transdermal, and parenteral administration because self-administration is simple, the lungs provide a large mucosal surface for drug absorption, there is no first-pass liver effect of absorbed drugs, and there is reduced enzymatic activity and pH mediated drug degradation compared with the oral route. Relatively high bioavailability of many molecules, including macromolecules, can be achieved via inhalation. Wall, D. A., Drug Delivery, 2: 1-20 1995); Patton, J. and Platz, R., Adv. Drug Del. Rev., 8: 179-196 (1992); and Byron, P., Adv. Drug. Del. Rev., 5: 107-132 (1990). As a result, several aerosol formulations of therapeutic drugs are in use or are being tested for delivery to the lung. Patton, J. S., et al., J. Controlled Release, 28: 79-85 (1994); Damms, B. and Bains, W., Nature Biotechnology (1996); Niven, R. W., et al., Pharm. Res., 12(9): 1343-1349 (1995); and Kobayashi, S., et al., Pharm. Res., 13(1): 80-83 (1996).

Drugs currently administered by inhalation come primarily as liquid aerosol formulations. However, many drugs and excipients, especially proteins, peptides (Liu, R., et al., Biotechnol. Bioeng., 37: 177-184 (1991)), and biodegradable carriers such as poly(lactide-co-glycolides) (PLGA), are unstable in aqueous environments for extended periods of time. This can make storage as a liquid formulation problematic. In addition, protein denaturation can occur during aerosolization with liquid formulations. Mumenthaler, M., et al., Pharm. Res., 11: 12-20 (1994). Considering these and other limitations, dry powder formulations (DPF's) are gaining increased interest as aerosol formulations for pulmonary delivery. Darnms, B. and W. Bains, Nature Biotechnology (1996); Kobayashi, S., et al, Pharm. Res., 13(1): 80-83 (1996); and Timsina, M., et al., Int. J. Pharm., 101: 1-13 (1994). However, among the disadvantages of DPF's is that powders of ultrafine particulates usually have poor flowability and aerosolization properties, leading to relatively low respirable fractions of aerosol, which are the fractions of inhaled aerosol that escape deposition in the mouth and throat. Gonda, I., in Topics in Pharmaceutical Sciences 1991, D. Crommelin and K. Midha, Editors, Stuttgart: Medpharm Scientific Publishers, 95-117 (1992). A primary concern with many aerosols is particulate aggregation caused by particle-particle interactions, such as hydrophobic, electrostatic, and capillary interactions.

An effective dry-powder inhalation therapy for both short and long term release of therapeutics, either for local or systemic delivery, utilizes a powder that displays minimum aggregation, as well as a means of avoiding or suspending the lung's natural clearance mechanisms until drugs have been effectively delivered.

One formulation for dry powder pulmonary delivery involves the separation of active particles from a carrier on actuation of the inhaler. Due to blending requirements, preparing these powders is associated with an increased number of steps. Furthermore, the method of delivery of these powders is associated with several disadvantages. For example, there are inefficiencies in the release of active particles from the carrier. Moreover, the carrier takes up significantly more volume than the active particle, thus high drug doses are difficult to achieve. In addition, the large lactose particles can impact the back of the throat, causing coughing.

As used herein, the term “microparticle” comprising iloprost and/or another pharmaceutical agent to be administered in addition to iloprost includes microspheres and microcapsules, as well as microparticles, unless otherwise specified. The term “microparticle” also includes the glassy formulations described herein. Microparticles may or may not be spherical in shape.

Several sustained release delivery systems for pharmaceutical agents delivered locally to the lung or for pharmaceutical agents delivered systemically through the lungs, have been developed. One such delivery system is a formulation comprising porous microparticles, where porosity, particle geometric diameter and composition are selected and used to control the rate of release of pharmaceutical agent from the microparticles following inhalation into the lungs. In particular, it has been discovered that the composition of the microparticles (e.g., the matrix material, surfactant) can be selected to provide delayed release (and avoid the burst effect associated with immediate release formulations), and the porosity of the microparticles can be selected to provide the majority of the pharmaceutical agent release before the microparticles are removed by the pulmonary clearance mechanisms. Although the composition of the microparticles can be selected to slow the release of the pharmaceutical agent, selection of the composition alone may not ensure that a sufficient amount of pharmaceutical agent is released before the microparticles are removed by the pulmonary clearance mechanisms. For a given composition of the microparticles, the porosity can be selected to ensure that a therapeutically or prophylactically effective amount of the pharmaceutical agent continues to be released after 2 hours, preferably such that a majority (e.g., more than about 20%, more than about 30%, more than about 40%, 50%, more than about 60%, more than about 75%, more than about 80% or more than about 90% by weight of the pharmaceutical agent) of the pharmaceutical agent is released from the microparticles by 24 hours following inhalation.

In some embodiments, the porous microparticles can provide sustained local delivery of pharmaceutical agent and/or sustained plasma levels without the need to complex the pharmaceutical agent molecule with another molecule. In addition, the sustained delivery formulations advantageously can moderate the pharmaceutical agent peaks and troughs associated with immediate release pharmaceutical agents, which can cause added toxicity or reduced efficacy.

In some embodiments, the sustained release formulations can deliver a majority of the inhaled microparticles to the appropriate region of the lung for the desired therapeutic or prophylactic use. That is, preferably, at least 50% by weight of the microparticles delivered to the lung is delivered, upon inhalation by the patient, to the appropriate region of the lung (for example, the combined central and upper lung) for the desired therapeutic or prophylactic use.

In some embodiments, the method and formulation can provide local or plasma concentrations at approximately constant values. For example, in some embodiments, they may not fluctuate by more than a factor of four over the period of sustained release.

As used herein, the terms “comprise,” “comprising,” “include,” and “including” are intended to be open, non-limiting terms, unless the contrary is expressly indicated.

The sustained release pharmaceutical formulations for pulmonary administration in accordance with one embodiment of the present invention include porous microparticles that comprise iloprost and/or another pharmaceutical agent to be administered in addition to iloprost and a matrix material. In some embodiments, the microparticle's composition, geometric diameter, and porosity provide that upon inhalation of the formulation into the lungs a therapeutically or prophylactically effective amount of iloprost and/or another pharmaceutical agent to be administered in addition to iloprost is released in a sustained manner from the microparticles in the lungs over a duration that extends up to at least about 2 hours, and preferably completes release by about 24 hours.

As a measure of the release rate, the mean absorption time following inhalation (NIATᵢₙₕ) for the drug can be used. The MATᵢₙₕ is the average time it takes for a drug molecule to be absorbed into the bloodstream from the lungs following inhalation and can be calculated from the pharmaceutical agent plasma profile following inhalation as follows:
MATᵢₙₕ=(AUMC_{inh∞}/AUC_{inh∞})−MRTᵢᵥ (EQ. 1)

where AUMCᵢₙₕ is area under the first moment curve (product of time and plasma concentration) from time zero to infinity following inhalation, AUCᵢₙₕ is the area under the plasma concentration curve from time zero to infinity following inhalation, and MRTᵢᵥ, is the mean residence time for the pharmaceutical agent of interest following intravenous administration.

The MRTᵢᵥ can be determined as follows:
MRTᵢᵥ=(AUMC_{iv∞}/AUC_{iv∞}) (EQ. 2)
where AUMCᵢᵥ is area under the first moment curve (product of time and plasma concentration) from time zero to infinity following intravenous administration, and AUCᵢᵥ is the area under the plasma concentration curve from time zero to infinity following intravenous administration.

For example, in some embodiments the porous microparticles can provide a mean absorption time for iloprost and/or another pharmaceutical agent to be administered in addition to iloprost following inhalation greater than that following inhalation when iloprost and/or another pharmaceutical agent to be administered in addition to iloprost is not delivered in microparticle form. The desired MATᵢₙₕ will depend on the drug molecule to be administered and the clinical indication, and it is helpful to consider the increase in MATᵢₙₕ obtained using the present microparticle formulations compared to the drug molecule when not delivered as microparticles. In some embodiments, a drug administered in microparticles of the present compositions and methods will provide an increase in MATᵢₙₕ of at least between about 25 and 50% as compared to the drug administered not in the present microparticles.

Formulations having a desired release profile are achieved by controlling microparticle composition, microparticle geometric size, and microparticle porosity. Porosity (ε) is the ratio of the volume of voids contained in the microparticles (Vᵥ) to the total volume of the microparticles (Vₜ).
ε=Vᵥ/Vₜ (EQ. 3)

This relationship can be expressed in terms of the envelope density (pₑ) of the microparticles and the absolute density (pₐ) of the microparticles:
ε=1−ρₑ/ρₐ (EQ. 4)

The absolute density is a measurement of the density of the solid material present in the microparticles, and is equal to the mass of the microparticles (which is assumed to equal the mass of solid material, as the mass of voids is assumed to be negligible) divided by the volume of the solid material (i.e., excludes the volume of voids contained in the microparticles and the volume between the microparticles). Absolute density can be measured using techniques such as helium pycnometry. The envelope density is equal to the mass of the microparticles divided by the volume occupied by the microparticles (i.e., equals the sum of the volume of the solid material and the volume of voids contained in the microparticles and excludes the volume between the microparticles). Envelope density can be measured using techniques such as mercury porosimetry or using a GeoPyc™ instrument (Micromeritics, Norcross, Ga.).

However, such methods are limited to geometric particle sizes larger than desirable for pulmonary applications. The envelope density can be estimated from the tap density of the microparticles. The tap density is a measurement of the packing density and is equal to the mass of microparticles divided by the sum of the volume of solid material in the microparticles, the volume of voids within the microparticles, and the volume between the packed microparticles of the material. Tap density (pt) can be measured using a GeoPyc™ instrument or techniques such as those described in the British Pharmacopoeia and ASTM standard test methods for tap density. It is known in the art that the envelope density can be estimated from the tap density for essentially spherical microparticles by accounting for the volume between the microparticles:
ρₑ=ρₜ0.794 (EQ. 5)

The porosity can be expressed as follows:
ε=1−ρₜ/(0.794*ρₐ) (EQ. 6)

For a given microparticle composition (pharmaceutical agent and matrix material) and structure (microparticle porosity and thus density) an iterative process can be used to define where the microparticles go in the lung and the duration over which the microparticles release the pharmaceutical agent: (1) the matrix material, the pharmaceutical agent content, and the microparticle geometric size are selected to determine the time and amount of initial pharmaceutical agent release; (2) the porosity of the microparticles is selected to adjust the amount of initial pharmaceutical agent release, and to ensure that significant release of the pharmaceutical agent occurs beyond the initial release and that the majority of the pharmaceutical agent release occurs within 24 hours; and then (3) the geometric particle size and the porosity are adjusted to 9 achieve a certain aerodynamic diameter which enables the particles to be deposited by inhalation to the region of interest in the lung.

As used herein, the term “initial release” refers to the amount of pharmaceutical agent released shortly after the microparticles become wetted. The initial release upon wetting of the microparticles results from pharmaceutical agent which is not fully encapsulated and/or pharmaceutical agent which is located close to the exterior surface of the microparticle. The amount of pharmaceutical agent released in the first 10 minutes is used as a measure of the initial release.

As used herein, the terms “diameter” or “d” in reference to particles refers to the number average particle size, unless otherwise specified. An example of an equation that can be used to describe the number average particle size is shown below:

$$d = \frac{{\sum }_{i = 1}^{ρ} {n}_{i} {d}_{i}}{{\sum }_{i = 1}^{ρ} {n}_{i}} \qquad ( EQ . 7 )$$

where n=number of particles of a given diameter (d).

As used herein, the terms “geometric size”, “geometric diameter, “volume average size,” “volume average diameter” or “d_{g}” refers to the volume weighted diameter average. An example of equations that can be used to describe the volume average diameter is shown below:

$${d}_{g} = {[ \frac{{\sum }_{i = 1}^{ρ} {n}_{i} {d}_{i}^{3}}{{\sum }_{i = 1}^{ρ} {n}_{i}} ]}^{1 / 3} \qquad ( EQ . 8 )$$

where n=number of particles of a given diameter (d).

As used herein, the term “volume median” refers to the median diameter value of the “volume-weighted” distribution. The median is the diameter for which 505 of the total are smaller and 50% are larger and corresponds to a cumulative fraction of 50%.

Geometric particle size analysis can be performed on a Coulter counter, by light scattering, by light microscopy, scanning electron microscopy, or transmittance electron microscopy, as known in the art. It is a generally held belief that the ideal scenario for delivery to the lung is to have an aerodynamic diameter<5 micrometers. See, e.g., Edwards et al., J. Appl. Physiol. 85(2):379-85 (1998); Suarez & Hickey, Respir. Care, 45(6):652-66 (2000); incorporated herein in its entirety by reference.

As used herein, the term “aerodynamic diameter” refers to the equivalent diameter of a sphere with density of 1 g/mL were it to fall under gravity with the same velocity as the particle analyzed. The aerodynamic diameter (dₐ) of a microparticle is related to the geometric diameter (d_{g}) and the envelope density (pₑ) by the following:
da=dg\sqrt{ρe} (EQ. 9)

Porosity affects envelope density (EQ. 4) which in turn affects aerodynamic diameter. Thus porosity can be used to affect both where the microparticles go in the lung and the rate at which the microparticles release the pharmaceutical agent in the lung. Gravitational settling (sedimentation), inertial impaction, Brownian diffusion, interception and electrostatic precipitation affect particle deposition in the lungs. Gravitational settling and inertial impaction are dependent on dₐ and are the most important factors for deposition of particles with aerodynamic diameters between 1 μm and 10 μm. Particles with dₐ>10 μm will not penetrate the tracheobronchial tree, particles with dₐ in the 3-10 μm range have predominantly tracheobronchial deposition, particles with dₐ in the 1-3 μm range are deposited in the alveolar region (deep lung), and particles with dₐ<1 μm are mostly exhaled. Respiratory patterns during inhalation can shift these aerodynamic particle size ranges slightly. For example, with rapid inhalation, the tracheobronchial region shifts to between 3 μm and 6 μm. It is a generally held belief that the ideal scenario for delivery to the lung is to have dₐ<5 μm. See, e.g., Edwards et al., J. Appl. Physiol. 85(2):379-85 (1998); Suarez & Hickey, Respir. Care, 45(6):652-66 (2000).

Aerodynamic particle size analysis can be performed via cascade impaction, liquid impinger analysis, or time-of-flight methods, as known in the art.

In some embodiments, the microparticles comprising iloprost and/or another pharmaceutical agent to be administered in addition to iloprost comprise a matrix material. The matrix material may be a structure including one or more materials in which the iloprost and/or another pharmaceutical agent to be administered in addition to iloprost are dispersed, entrapped, or encapsulated. The matrix is preferably in the form of porous microparticles. Optionally, the porous microparticles further include one or more surfactants.

As used herein, the term “microparticle” comprising iloprost and/or another pharmaceutical agent to be administered in addition to iloprost includes microspheres and microcapsules, as well as microparticles, unless otherwise specified. Microparticles may or may not be spherical in shape. Microcapsules are defined as microparticles having an outer shell surrounding a core containing another material, for example, the pharmaceutical agent. Microspheres comprising pharmaceutical agent and matrix can be porous having a honeycombed structure or a single internal void. Either type of microparticle may also have pores on the surface of the microparticle.

In one embodiment, the microparticles comprising iloprost and/or another pharmaceutical agent to be administered in addition to iloprost have a volume average diameter between 0.1 and 5 micrometers (e.g., between 1 and 5 micrometers, between 2 and 5 micrometers, etc.). In another embodiment, the microparticles have a volume average diameter of up to 10 micrometers, for targeting delivery to the large bronchi. Particle size (geometric diameter and aerodynamic diameter) is selected to provide an easily dispersed powder that upon aerosolization and inhalation readily deposits at a targeted site in the respiratory tract (e.g., upper airway, deep lung, etc.), preferably while avoiding or minimizing excessive deposition of the particles in the oropharyngel or nasal regions. In one preferred embodiment, the porous microparticles have a volume average diameter of between 2 and 5 micrometers. The volume average diameter is also selected to avoid and minimize effects of one of the lung's natural clearance mechanisms (e.g. phagocytosis by macrophages). Generally, larger particles are phagocytosed at a slower rate.

In one embodiment, the microparticles comprising iloprost and/or another pharmaceutical agent to be administered in addition to iloprost have an average porosity between about 15 and 90%. The porosity of the microparticles is preferably selected so that the majority of the pharmaceutical agent is released before the particle is removed from the lung by biological clearance mechanisms such as mucociliary clearance. In specific embodiments, the average porosity can be between about 25 and about 75%, between about 35 and about 65%, or between about 40 and about 60%.

Matrix Material

The matrix material is a material that functions to slow down release of the pharmaceutical agent from the microparticle. It can be formed of non-biodegradable or biodegradable materials, although biodegradable materials are preferred, particularly for inhalation administration.

The matrix material can be crystalline, semi-crystalline, or amorphous. The matrix material may be a polymer, a lipid, a salt, a hydrophobic small molecule, or a combination thereof. In some embodiments the matrix material is a lipid such that the microparticle is a liposome.

The iloprost and/or another pharmaceutical agent to be administered in addition to iloprost can be present in the porous microparticle in an amount that is greater than or less than the amount of matrix material that is present in the porous microparticle, depending upon the particular formulation needs.

In some embodiments, the matrix material comprises at least 5% w/w of the microparticle. In some embodiments, the content of matrix material in the microparticles can be between 5 and about 95 wt %. In typical embodiments, the matrix material is present in an amount between about 50 and 90 wt %.

Representative synthetic polymers include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polyalkylenes such as polyethylene and polypropylene, polyalkylene glycols such as poly(ethylene glycol), polyalkylene oxides such as poly(ethylene oxide), polyvinyl alcohols, polyvinyl ethers, polyvinylpyrrolidone, poly(butyric acid), poly(valeric acid), and poly(lactide-co-caprolactone), copolymers, derivatives, and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxy acids such as lactic acid and glycolic acid (including poly(lactide-co-glycolide)), and copolymers with PEG, polyanhydrides, poly(ortho)esters, poly(butyric acid), poly(valeric acid), poly(lactide-co-caprolactone), blends and copolymers thereof.

Examples of preferred natural polymers include proteins such as albumin, fibrinogen, gelatin, and prolamines, for example, zein, and polysaccharides such as alginate, cellulose and polyhydroxyalkanoates, for example, polyhydroxybutyrate.

Representative lipids include the following classes of molecules: fatty acids and derivatives, mono-, di- and triglycerides, phospholipids, sphingolipids, cholesterol and steroid derivatives, terpenes, and vitamins. Fatty acids and derivatives thereof may include saturated and unsaturated fatty acids, odd and even number fatty acids, cis and trans isomers, and fatty acid derivatives including alcohols, esters, anhydrides, hydroxy fatty acids and prostaglandins. Saturated and unsaturated fatty acids that may be used include molecules that have between 12 carbon atoms and 22 carbon atoms in either linear or branched form. Examples of saturated fatty acids that may be used include lauric, myristic, palmitic, and stearic acids. Examples of unsaturated fatty acids that may be used include lauric, physeteric, myristoleic, palmitoleic, petroselinic, and oleic acids. Examples of branched fatty acids that may be used include isolauric, isomyristic, isopalmitic, and isostearic acids and isoprenoids. Fatty acid derivatives include 12(((7′-diethylaminocoumarin-3 yl)carbonyl)methylamino)-octadecanoic acid; N-[12(((7′diethylaminocoumarin-3-yl) carbonyl)methyl-amino) octadecanoyl]-2-aminopalmitic acid, N succinyl-dioleoylphosphatidylethanol amine and palmitoyl-homocysteine; and/or combinations thereof. Mono, di- and triglycerides or derivatives thereof that may be used include molecules that have fatty acids or mixtures of fatty acids between 6 and 24 carbon atoms, digalactosyldiglyceride, 1,2-dioleoyl-snglycerol; 1,2-dipalmitoyl-sn-3 succinylglycerol; and 1,3-dipalmitoyl succinylglycerol.

In one preferred embodiment, the matrix material comprises a phospholipid or combinations of phospholipids. Phospholipids that may be used include phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phosphatidylcholines include such as dioleoylphosphatidylcholine, dimyristoylphosphatidylcholine (DUTC), dipentadecanoylphosphatidylcholine dilauroylphosphatidylcholine, dipalmitoylphosphatidylcholine (DPPC), distearoylphosphatidylcholine (DSPQ, diarachidoylphosphatidylcholine (DAPQ, dibehenoylphosphatidylcholine (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) may also be used. Examples of phosphatidylethanolamines include dicaprylphosphatidylethanolamine, dioctanoylphosphatidylethanolamine, dilauroylphosphatidylethanolamine, dimyristoylphosphatidylethanolamine (DMPE), dipalmitoylphosphatidylethanolamine (DPPE), dipalmitoleoylphosphatidylethanolamine, distearoylphosphatidylethanolamine (DSPE), dioleoylphosphatidylethanolamine, and dilineoylphosphatidylethanolamine. Examples of phosphatidylglycerols include dicaprylphosphatidylglycerol, dioctanoylphosphatidylglycerol, dilauroylphosphatidylglycerol, dimyristoylphosphatidylglycerol (DMPG), dipalmitoylphosphatidylglycerol (DPPG), dipalmitoleoylphosphatidylglycerol, distearoylphosphatidylglycerol (DSPG), dioleoylphosphatidylglycerol, and dilineoylphosphatidylglycerol. Preferred phospholipids include DMPC, DPPC, DAPC, DSPC, DTPC, DBPC, DMPG, DPPG, DSPG, DMPE, DPPE, and DSPE.

Additional examples of phospholipids include modified phospholipids for example phospholipids having their head group modified, e.g., alkylated or polyethylene glycol (PEG)-modified, hydrogenated phospholipids, phospholipids with multifarious head groups (phosphatidylmethanol, phosphatidylethanol, phosphatidylpropanol, phosphatidylbutanol, etc.), dibromo phosphatidylcholines, mono and diphytanoly phosphatides, mono and diacetylenic phosphatides, and PEG phosphatides.

Sphingolipids that may be used include ceramides, sphingomyelins, cerebrosides, gangliosides, sulfatides and lysosulfatides. Examples of sphinglolipids include the gangliosides GM1 and GM2.

Steroids which may be used include cholesterol, cholesterol sulfate, cholesterol hemisuccinate, 6-(5-cholesterol 3β-yloxy) hexyl amino deoxy-1-thio-(α-Dgalactopyranoside, 6-(5-cholesten-3β-yloxy)hexyl amino deoxyl-1-thio-α-D mannopyranoside and cholesteryl(4′-trimethyl 35 ammonio)butanoate.

Additional lipid compounds that may be used include tocopherol and derivatives, and oils and derivatized oils such as stearlyamine.

Other suitable hydrophobic compounds include amino acids such as tryptophane, tyrosine, isoleucine, leucine, and valine, aromatic compounds such as an alkyl paraben, for example, methyl paraben, tyloxapol, and benzoic acid.

The matrix may comprise pharmaceutically acceptable small molecules such as carbohydrates (including mono and disaccharides, sugar alcohols and derivatives of carbohydrates such as esters), and amino acids, their salts and their derivatives such as esters and amides.

A variety of cationic lipids such as DOTMA, N-[1-(2,3-dioleoyloxy)propylN,N,N-trimethylammonium chloride; DOTAP, 1,2-dioleoyloxy-3-(trimethylammonio) propane; and DOTB, 1,2-dioleoyl-3-(4′-trimethyl-ammonio) butanoyl-sn glycerol may be used.

Inorganic materials can be included in the microparticles. Salts of metals (inorganic salts), such as calcium chloride or sodium chloride may be present in the particle or used in the production of the particles. Metal ions such as calcium, magnesium, aluminum, zinc, sodium, potassium, lithium and iron may be used as the counterion for salts with organic acids such as citric acid and/or lipids including phospholipids. Examples of salts of organic acids include sodium citrate, sodium ascorbate, magnesium gluconate, and sodium gluconate. A variety of metal ions may be used in such complexes, including lanthanides, transition metals, alkaline earth metals, and mixtures of metal ions. Salts of organic bases may be included such as tromethamine hydrochloride.

In one embodiment, the microparticles may include one or more carboxylic acid as the free acid or the salt form. The salt can be a divalent salt. The carboxylate moiety can be a hydrophilic carboxylic acid or salt thereof. Suitable carboxylic acids include hydroxydicarboxylic acids, hydroxytricarboxilic acids and the like. Citric acid and citrate are preferred. Suitable counterions for salts include sodium and alkaline earth metals such as calcium. Such salts can be formed during the preparation of the particles, from the combination of one type of salt such as calcium chloride and carboxylic acid as the free acid or an alternative salt form such as the sodium salt.

Surfactants

In one embodiment, the porous microparticles further include one or more surfactants. As used herein, a “surfactant” is a compound that is hydrophobic or amphiphilic (i.e., including both a hydrophilic and a hydrophobic component or region). Surfactants can be used to facilitate microparticle formation, to modify the surface properties of the microparticles and alter the way in which the microparticles are dispersed with a dry powder inhalation device or a metered dose inhaler, to alter the properties of the matrix material (e.g. to increase or decrease the hydrophobicity of the matrix), or to perform a combination of functions thereof. It is to be distinguished from similar or identical materials forming the “matrix material.” The content of surfactant in the porous microparticles generally is less than about 10% by weight of the microparticles.

In one embodiment, the surfactant comprises a lipid. Lipids that may be used include the following classes of lipids: fatty acids and derivatives, mono-, di- and triglycerides, phospholipids, sphingolipids, cholesterol and steroid derivatives, terpenes, prostaglandins and vitamins. Fatty acids and derivatives thereof may include saturated and unsaturated fatty acids, odd and even number fatty acids, cis and trans isomers, and fatty acid derivatives including alcohols, esters, anhydrides, hydroxy fatty acids, and salts of fatty acids. Saturated and unsaturated fatty acids that may be used include molecules that have between 12 carbon atoms and 22 carbon atoms in either linear or branched form. Examples of saturated fatty acids that may be used include lauric, myristic, palmitic, and stearic acids. Examples of unsaturated fatty acids that may be used include lauric, physeteric, myristoleic, palmitoleic, petroselinic, and oleic acids. Examples of branched fatty acids that may be used include isolauric, isomyristic, isopalmitic, and isostearic acids and isoprenoids. Fatty acid derivatives include 12(((7′-diethylaminocoumarin-3 yl)carbonyl)methylamino)-octadecanoic acid; N-[12(((7′diethylaminocoumarin-3 yl) carbonyl)methyl-amino) octadecanoyl]aminopalmitic acid, N succinyl-dioleoylphosphatidylethanol amine and palmitoylhomocysteine; and/or combinations thereof. Mono, di- and triglycerides or derivatives thereof that may be used include molecules that have fatty acids or mixtures of fatty acids between 6 and 24 carbon atoms, digalactosyldiglyceride, 1,2-dioleoyl-snglycerol; 1,2-dipalmitoyl-sn-3 succinylglycerol; and 1,3-dipalmitoyl-2-succinylglycerol.

In one preferred embodiment, the surfactant comprises a phospholipid. Phospholipids that may be used include phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phosphatidylcholines include such as dioleoylphosphatidylcholine, dimyristoylphosphatidylcholine (DMPC), dipentadecanoylphosphatidylcholine dilauroylphosphatidylcholine, dipalmitoylphosphatidylcholine (DPPQ, distearoylphosphatidylcholine (DSPQ, diarachidoylphosphatidylcholine (DAPC), dibehenoylphosphatidylcholine (DBPQ, ditricosanoylphosphatidylcholine (DTPQ, dilignoceroylphatidylcholine (DLPC); and phosphatidylethanolamines such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) may also be used. Examples of phosphatidylethanolamines include dicaprylphosphatidylethanolamine, dioctanoylphosphatidylethanolamine, dilauroylphosphatidylethanolamine, dimyristoylphosphatidylethanolamine (DMPE), dipalmitoylphosphatidylethanolamine (DPPE), dipalmitoleoylphosphatidylethanolamine, distearoylphosphatidylethanolamine (DSPE), dioleoylphosphatidylethanolamine, and dilineoylphosphatidylethanolamine. Examples of phosphatidylglycerols include dicaprylphosphatidylglycerol, dioctanoylphosphatidylglycerol, dilauroylphosphatidylglycerol, dimyristoylphosphatidylglycerol (DMPG), dipalmitoylphosphatidylglycerol (DPPG), dipalmitoleoylphosphatidylglycerol, distearoylphosphatidylglycerol (DSPG), dioleoylphosphatidylglycerol, and dilineoylphosphatidylglycerol. Preferred phospholipids include DMPC, DPPC, DAPC, DSPC, DTPC, DBPC, DLPC, DMPG, DPPG, DSPG, DMPE, DPPE, and DSPE, and most preferably DPPC, DAPC and DSPC.

Sphingolipids that may be used include ceramides, sphingomyelins, cerebrosides, gangliosides, sulfatides and lysosulfatides. Examples of sphinglolipids include the gangliosides GM1 and GM2.

Steroids which may be used include cholesterol, cholesterol sulfate, cholesterol hemisuccinate, 6-(5-cholesterol 3β-yloxy) hexyl-6-amino-6-deoxy-1-thio-α-Dgalactopyranoside, 6-(5-cholesten-3-yloxy)hexyl-6-amino-6-deoxy]-1-thio-(α-D mannopyranoside and cholesteryl(4′-trimethyl 35 ammonio)butanoate.

Additional lipid compounds that may be used include tocopherol and derivatives, and oils and derivatized oils such as stearlyamine.

A variety of cationic lipids such as DOTMA, N-[1-(2,3-dioleoyloxy)propylN,N,N-trimethylammonium chloride; DOTAP, 1,2-dioleoyloxy-3-(trimethylammonio) propane; and DOTB, 1,2-dioleoyl-3-(4′-trimethyl-ammonio) butanoyl-sn glycerol may be used.

A variety of other surfactants may be used including ethoxylated sorbitan esters, sorbitan esters, fatty acid salts, sugar esters, pluronics, tetronics, ethylene oxides, butylene oxides, propylene oxides, anionic surfactants, cationic surfactants, mono and diacyl glycerols, mono and diacyl ethylene glycols, mono and diacyl sorbitols, mono and diacyl glycerol succinates, alkyl acyl phosphatides, fatty alcohols, fatty amines and their salts, fatty ethers, fatty esters, fatty amides, fatty carbonates, cholesterol esters, cholesterol amides and cholesterol ethers.

Examples of anionic or cationic surfactants include aluminum monostearate, ammonium lauryl sulfate, calcium stearate, dioctyl calcium sulfosuccinate, dioctyl potassium sulfosuccinate, dioctyl sodium sulfosuccinate, emulsifying wax, magnesium lauryl sulfate, potassium oleate, sodium caster oil, sodium cetostearyl sulfate, sodium lauryl ether sulfate, sodium lauryl sulfate, sodium lauryl sulfbacetate, sodium oleate, sodium stearate, sodium stearyl fumarate, sodium tetradecyl sulfate, zinc oleate, zinc stearate, benzalconium chloride, cetrimide, cetrimide bromide, and cetylpyridinium chloride.

Iloprost or Other Pharmaceutical Agent to be Administered in Addition to Iloprost

The iloprost and/or another pharmaceutical agent to be administered in addition to iloprost may be provided in any form suitable for administration to the desired subject. For example, the iloprost and/or another pharmaceutical agent to be administered in addition to iloprost may be present in an amorphous state, a crystalline state, or a mixture thereof.

In addition, the iloprost and/or another pharmaceutical agent to be administered in addition to iloprost may be provided in alternative salt forms, free acid forms, free base forms, and hydrates.

In some embodiments, the content of pharmaceutical agent in the microparticles is between about 1 and about 70 wt %. In other embodiments, the pharmaceutical agent is present in an amount between about 5 and 50 wt %.

In one embodiment, microparticles comprise iloprost and another pharmaceutical agent. In one embodiment, the iloprost and the other pharmaceutical agent are combined into and delivered from one microparticle In another embodiment, the formulation comprises a mixture of two or more different microparticles, one of which contains the iloprost and the others of which contain the other pharmaceutical agent or pharmaceutical agents to be administered in addition to iloprost. In one embodiment, the formulation includes at least one pharmaceutical agent for sustained release and at least one other pharmaceutical agent for immediate release. Thus, either the iloprost or the other pharmaceutical agents to be administered in addition to iloprost can be provided in sustained release or immediate release form.

In yet another embodiment, the microparticle formulations comprise a mixture of different microparticles each containing a single pharmaceutical agent (either iloprost or another pharmaceutical agent to be administered in addition to iloprost, but having different porosities, so that some particles of the mixture have a first release profile (e.g., a majority of the first pharmaceutical agent is released between 2 and 6 hours) and other particles have a second pharmaceutical agent release profile (e.g., a majority of the second pharmaceutical agent is released between 6 and 12 hours, or between 6 and 24 hours).

Materials to Inhibit Uptake by the RES

In some embodiments, uptake and removal of the microparticles by macrophages can be slowed or minimized through increasing the geometric particle size (e.g., >3 micrometers slows phagocytosis) the selection of the polymer and/or incorporation or coupling of molecules that minimize adhesion or uptake or by incorporating the poly(alkylene glycol) into the matrix such that at least one glycol unit is surface exposed. For example, tissue adhesion by the microparticles can be minimized by covalently binding poly(alkylene glycol) moieties to the surface of the microparticle. The surface poly(alkylene glycol) moieties have a high affinity for water that reduces protein adsorption onto the surface of the particle. The recognition and uptake of the microparticles by the reticulo-endothelial system (RES) is therefore reduced.

In one method, the terminal hydroxyl group of the poly(alkylene glycol) is covalently attached to biologically active molecules, or molecules affecting the charge, lipophilicity or hydrophilicity of the particle, onto the surface of the microparticle.

Methods available in the art can be used to attach any of a wide range of ligands to the microparticles to enhance the delivery properties, the stability or other properties of the microparticles in vivo.

Bulking Agents

In some embodiments for administration to the pulmonary system using a dry powder inhaler, the porous microparticles can be combined (e.g., blended) with one or more pharmaceutically acceptable bulking agents and administered as a dry powder.

Examples of pharmaceutically acceptable bulking agents include sugars such as mannitol, sucrose, lactose, fructose and trehalose and amino acids. Amino acids that can be used include glycine, arginine, histidine, threonine, asparagine, aspartic acid, serine, glutamate, proline, cysteine, methionine, valine, leucine, isoleucine, tryptophan, phenylalanine, tyrosine, lysine, alanine, and glutamine. In one embodiment, the bulking agent comprises particles having a volume average size between 10 and 500 micrometers.

Suspending Agents

In some embodiments for administration to the pulmonary system, the porous microparticles can be suspended with one or more pharmaceutically acceptable suspending agents that are liquid, within a metered dose inhaler and administered via a metered dose inhaler.

Examples of pharmaceutically acceptable suspending agents include chlorofluorocarbons and hydrofluorocarbons. Examples of pharmaceutically acceptable suspending agent for use in metered dose inhalers include hydrofluorocarbons (such as HFA-134a and HFA-227) and chlorofluorocarbons (such as CFC-11, CFC-12, and CFC-114). Mixtures of suspending agents can be used.

Making the Porous Microparticles

In some embodiments, the porous microparticles are made by a method that includes the following steps: (1) dissolving the matrix material in a volatile solvent to form a matrix material solution; (2) adding the iloprost and/or other pharmaceutical agent to be administered in addition to iloprost to the solution of matrix material; (3) optionally combining at least one pore forming agent with the iloprost and/or other pharmaceutical agent to be administered in addition to iloprost in the matrix material solution and emulsifying to form an emulsion, suspension, or second solution; and (4) removing the volatile solvent, and the pore forming agent if present, from the emulsion, suspension, or second solution to yield porous microparticles which comprise the iloprost and/or other pharmaceutical agent to be administered in addition to iloprost and the matrix material. In some embodiments, the microparticles provide sustained release of the iloprost and/or another pharmaceutical agent to be administered in addition to iloprost. For example, in some embodiments, the method produces microparticles that upon inhalation of the formulation into the lungs release a therapeutically or prophylactically effective amount of the iloprost and/or other pharmaceutical agent to be administered in addition to iloprost from the microparticles in the lungs for at least 2 hours.

Techniques that can be used to make the porous microparticles include melt extrusion, spray drying, fluid bed drying, solvent extraction, hot melt encapsulation, and solvent evaporation, as discussed below. In one embodiment, microparticles are produced by spray drying. The iloprost and/or other pharmaceutical agent to be administered in addition to iloprost can be incorporated into the matrix as solid particles, liquid droplets, or by dissolving the iloprost and/or other pharmaceutical agent to be administered in addition to iloprost in the matrix material solvent. If the iloprost and/or other pharmaceutical agent to be administered in addition to iloprost is a solid, it may be encapsulated as solid particles which are added to the matrix material solution or may be dissolved in an aqueous solution which then is emulsified with the matrix material solution prior to encapsulation, or the solid iloprost and/or other pharmaceutical agent to be administered in addition to iloprost may be cosolubilized together with the matrix material in the matrix material solvent.

In one embodiment, the method further comprises combining one or more surfactants, with the pharmaceutical agent in a matrix material solution. In one embodiment of the methods for making microparticles, the process further includes blending the porous microparticles with a pharmaceutically acceptable bulking agent.

In one example, the matrix material comprises a biocompatible synthetic polymer, and the volatile solvent comprises an organic solvent. In another example, the pore forming agent is in the form of an aqueous solution when combined with the pharmaceutical agent/matrix solution.

In one embodiment, the step of removing the volatile solvent and pore forming agent from the emulsion, suspension, or second solution is conducted using a process selected from spray drying, evaporation, fluid bed drying, lyophilization, vacuum drying, or a combination thereof.

Solvent Evaporation

In this method, the matrix material and pharmaceutical agent are dissolved in a volatile organic solvent such as methylene chloride. A pore forming agent as a solid or as a liquid may be added to the solution. The active agent can be added as either a solid or in solution to the polymer solution. The mixture is sonicated or homogenized and the resulting dispersion or emulsion is added to an aqueous solution that may contain a surface active agent such as TWEEN™20, TWEEN™80, PEG or poly(vinyl alcohol) and homogenized to form an emulsion. The resulting emulsion is stirred until most of the organic solvent evaporates, leaving microparticles. Microparticles with different geometric sizes and morphologies can be obtained by this method by controlling the emulsion droplet size. Solvent evaporation is described by Mathiowitz, et al., J. Scanning Microscop, 4:329 (1990); Beck, et al., Fertil. Steril., 31:545 (1979); and 26 Benita, et al., J. Pharm. Sci., 73:1721 (1984).

Particularly hydrolytically unstable polymers, such as polyanhydrides, may degrade during the fabrication process due to the presence of water. For these polymers, the following two methods, which are performed in completely organic solvents, are more useful.

Hot Melt Microencapsulation

In this method, the matrix material and the pharmaceutical agent are first melted and then mixed with the solid or liquid active agent. A pore forming agent as a solid or in solution may be added to the solution. The mixture is suspended in a non-miscible solvent (like silicon oil), and, while stirring continuously, heated to 5 degrees C. above the melting point of the polymer. Once the emulsion is stabilized, it is cooled until the polymer particles solidify. The resulting microparticles are washed by decantation with a polymer non-solvent such as petroleum ether to give a free-flowing powder. Hot-melt microencapsulation is described by Mathiowitz, et al., Reactive Polymers, 6:275 (1987).

Solvent Removal

This technique was primarily designed for hydrolytically unstable materials. In this method, the solid or liquid iloprost and/or other pharmaceutical agent to be administered in addition to iloprost is dispersed or dissolved in a solution of the selected matrix material and pharmaceutical agent in a volatile organic solvent like methylene chloride. This mixture is suspended by stirring in an organic oil (such as silicon oil) to form an emulsion. The external morphology of particles produced with this technique is highly dependent on the type of polymer used.

Spray Drying of Microparticles

Microparticles can be produced by spray drying by a method that includes the following steps: (1) dissolving the matrix material, and optionally a surfactant, in a volatile solvent to form a matrix material solution; (2) adding iloprost or another pharmaceutical agent to be administered in addition to iloprost to the solution of matrix material; (3) optionally combining at least one pore forming agent with the iloprost and/or other pharmaceutical agent to be administered in addition to iloprost in the matrix material solution; (4) forming an emulsion, suspension or second solution from the iloprost and/or other pharmaceutical agent to be administered in addition to iloprost, the matrix material solution, and the optional pore forming agent; and (5) spray drying the emulsion, suspension or solution and removing the volatile solvent and the pore forming agent, if present, to form porous microparticles. As defined herein, the process of “spray drying” an emulsion, suspension or solution containing a matrix material and iloprost or another pharmaceutical agent to be administered in addition to iloprost refers to a process wherein the emulsion, suspension or solution is atomized to form a fine mist and dried by direct contact with temperature-controlled carrier gases. In a typical embodiment using spray drying apparatus available in the art, the emulsion, suspension or solution is delivered through the inlet port of the spray drier, passed through a tube within the drier and then atomized through the outlet port. The temperature may be varied depending on the gas or matrix material used. The temperature of the inlet and outlet ports can be controlled to produce the desired products.

The geometric size of the particulates formed is a function of the atomizer used to spray the matrix material solution, atomizer pressure, the flow rate, the matrix material used, the matrix material concentration, the type of solvent and the temperature of spraying (both inlet and outlet temperature). Microparticles ranging in geometric diameter between one and ten microns can be obtained.

If the iloprost and/or other pharmaceutical agent to be administered in addition to iloprost is a solid, the iloprost and/or other pharmaceutical agent to be administered in addition to iloprost may be encapsulated as solid particles which are added to the matrix material solution prior to spraying, or the iloprost and/or other pharmaceutical agent to be administered in addition to iloprost can be dissolved in a solvent which then is emulsified with the matrix material solution prior to spraying, or the solid may be cosolubilized together with the matrix material in an appropriate solvent prior to spraying.

Reagents for Making the Porous Microparticles

Certain reagents used to make the porous microparticles may include solvents for the matrix material, solvents or vehicles for the iloprost and/or other pharmaceutical agent to be administered in addition to iloprost, pore forming agents, and various additives to facilitate microparticle formation.

Solvents

A solvent for the matrix material is selected based on its biocompatibility as well as the solubility of the matrix material and where appropriate, interaction with the iloprost and/or other pharmaceutical agent to be administered in addition to iloprost to be delivered. For example, the ease with which the matrix material is dissolved in the solvent and the lack of detrimental effects of the solvent on the pharmaceutical agent to be delivered are factors to consider in selecting the matrix material solvent. Aqueous solvents can be used to make matrices formed of watersoluble polymers. Organic solvents will typically be used to dissolve hydrophobic and some hydrophilic matrix materials. Combinations of aqueous and organic solvents may be used. Preferred organic solvents are volatile or have a relatively low boiling point or can be removed under vacuum and which are acceptable for administration to humans in trace amounts, such as methylene chloride. Other solvents, such as ethyl acetate, ethanol, methanol, dimethyl fortnamide (DMF), acetone, acetonitrile, tetrahydrofuran (THF), acetic acid, dimethyl sulfoxide (DMSO) and chloroform, and combinations thereof, also may be utilized. Preferred solvents are those rated as class 3 residual solvents by the Food and Drug Administration, as published in the Federal Register vol. 62. number 85, pp. 24301-09 (May 1997).

In general, the matrix material is dissolved in the solvent to form a matrix material solution having a concentration of between 0.1 and 60% weight to volume (w/v), more preferably between 0.25 and 30%. The matrix material solution is then processed as described below to yield a matrix having iloprost and/or other pharmaceutical agents to be administered in addition to iloprost incorporated therein.

Surfactants to Facilitate Microparticle Formation

A variety of surfactants may be added to a solution, suspension, or emulsion containing matrix material to facilitate microparticle formation. The surfactants may be added to any phase of an emulsion as emulsifiers if an emulsion is used during the production of the matrices. Exemplary emulsifiers or surfactants that may be used (e.g., between about 0.1 and 5% by weight relative to weight of the iloprost and/or other pharmaceutical agent to be administered in addition to iloprost and matrix material) include most physiologically acceptable emulsifiers. Examples include natural and synthetic forms of bile salts or bile acids, both conjugated with amino acids and unconjugated such as taurodeoxycholate, and cholic acid. Phospholipids can be used as mixtures, including natural mixtures such as lecithins. These surfactants may function solely as emulsifiers, and as such form part of and are dispersed throughout the matrix of the particles.

Additives to Facilitate Microparticle Dispersion

The composition of the microparticles may comprise a surfactant in a manner such that the microparticles will have all or part of the surfactant structure surface exposed, and as such will facilitate dispersion of the microparticles for administration via dry powder inhaler or via metered dose inhaler. Surfactants for facilitating dispersion may be included during production of the microparticles. Alternatively, the microparticles may be coated with the surfactant post-production. Exemplary surfactants that may be used (e.g., between about 0.1 and 5% by weight relative to weight of the iloprost and/or other pharmaceutical agent to be administered in addition to iloprost and matrix material) include phospholipids, salts of fatty acids, and molecules containing PEG units such as polysorbate 80.

Control of Porosity

The porosity of the microparticles can be controlled during the production of the microparticles by adjusting the solids content of the iloprost and/or other pharmaceutical agent to be administered in addition to iloprost in matrix material solution or adjusting the rate at which the matrix solvent is removed, or combinations thereof. Higher solids concentrations lead to microparticles with less porosity.

Alternatively, pore forming agents as described below can be used to control the porosity of the microparticles during production. Pore forming agents are volatile materials that are used during the process to create porosity in the resultant matrix. The pore forming agent can be a volatilizable solid or volatilizable liquid.

Liquid Pore Forming Agent

The liquid pore forming agent must be immiscible with the matrix material solvent and volatilizable under processing conditions compatible with the iloprost and/or other pharmaceutical agent to be administered in addition to iloprost and matrix material. To effect pore formation, the pore forming agent first is emulsified with the iloprost and/or other pharmaceutical agent to be administered in addition to iloprost in the matrix material solution. Then, the emulsion is further processed to remove the matrix material solvent and the pore forming agent simultaneously or sequentially using evaporation, vacuum drying, spray drying, fluid bed drying, lyophilization, or a combination of these techniques.

The selection of liquid pore forming agents will depend on the matrix material solvent. Representative liquid pore forming agents include water; dichloromethane; alcohols such as ethanol, methanol, or isopropanol; acetone; ethyl acetate; ethyl formate; dimethylsulfoxide; acetonitrile; toluene; xylene; dimethylforamide; ethers such as THF, diethyl ether, or dioxane; triethylatnine; foramide; acetic acid; methyl ethyl ketone; pyridine; hexane; pentane; furan; water; liquid perfluorocarbons, and cyclohexane.

The liquid pore forming agent is used in an amount that is between about 1 and about 50% (v/v), preferably between about 5 and about 25% (v/v), of the pharmaceutical agent solvent emulsion.

Solid Pore Forming Agent

The solid pore forming agent must be volatilizable under processing conditions which do not harm the iloprost and/or other pharmaceutical agent to be administered in addition to iloprost or matrix material. The solid pore forming agent can be (i) dissolved in the matrix material solution which contains the iloprost and/or other pharmaceutical agent to be administered in addition to iloprost, (ii) dissolved in a solvent which is not miscible with the matrix material solvent to form a solution which is then emulsified with the matrix material solution which contains the iloprost and/or other pharmaceutical agent to be administered in addition to iloprost, or (iii) added as solid particulates to the matrix material solution which contains the iloprost and/or other pharmaceutical agent to be administered in addition to iloprost. The solution, emulsion, or suspension of the pore forming agent in the iloprost and/or other pharmaceutical agent to be administered in addition to iloprost/matrix material solution then is further processed to remove the matrix material solvent, the pore forming agent, and, if appropriate, the solvent for the pore forming agent simultaneously or sequentially using evaporation, spray drying, fluid bed drying, lyophilization, vacuum drying, or a combination of these techniques. After the matrix material is precipitated, the hardened microparticles can be frozen and lyophilized to remove any pore forming agents not removed during the microencapsulation process.

In some embodiments, the solid pore forming agent is a volatile salt, such as salts of volatile bases combined with volatile acids. Volatile salts are materials that can transform from a solid or liquid to a gaseous state using added heat and/or vacuum. Examples of volatile bases include ammonia, methylamine, ethylamine, dimethylamine, diethylamine, methylethylamine, trimethylamine, triethylamine, and pyridine. Examples of volatile acids include carbonic acid, hydrochloric acid, hydrobromic acid, hydroiodic acid, formic acid, acetic acid, propionic acid, butyric acid, and benzoic acid. Preferred volatile salts include ammonium bicarbonate, ammonium acetate, ammonium chloride, ammonium benzoate and mixtures thereof. Other examples of solid pore forming agents include iodine, phenol, benzoic acid (as acid not as salt), camphor, and naphthalene.

The solid pore forming agent is used in an amount between about 5 and about 1000% (w/w), preferably between about 10 and about 600% (w/w), and more preferably between about 10 and about 100% (w/w), of the iloprost and/or other pharmaceutical agent to be administered in addition to iloprost and the matrix material.

Methods of Administering the Porous Microparticles

The formulation comprising porous microparticles comprising iloprost as described herein preferably is administered to the lungs of a patient by oral inhalation, for example by having the patient inhale a dry powder form of the formulation using a suitable inhalation device. Dry powder inhalation devices for medicaments, which disperse the pharmaceutical agent in air or a propellant, are well known in the art. See, e.g., U.S. Pat. No. 5,327,883; No. 5,577,497; and No. 6,060,069, the disclosures of which are incorporated herein by reference in their entireties. Types of inhalation devices include dry powder inhalers (DPIs), metered dose inhalers (MDIs), and nebulizers. Commercial embodiments of some of these include the SPIROS™ DPI (Dura Pharmaceuticals, Inc. US), the ROTOHALER™, the TURBUHALER™ (Astra SE), the CYCLOHALER™ (Pharmachemie B.V.), FLOWCAPS™ (Hovione) and the VENTODISK™(Glaxo, UK). For administration to the pulmonary system using a dry powder inhaler, the porous microparticles can be combined (e.g., blended) with one or more pharmaceutically acceptable bulking agents and administered as a dry powder. Examples of pharmaceutically acceptable bulking agents include sugars such as mannitol, sucrose, lactose, fructose, and trehalose and amino acids.

In one embodiment, the formulation with or without bulking agent is loaded into a unit dose receptacle (e.g., a gelatin, hydropropylmethylcellose or plastic capsule, or blister) which is then placed within a suitable inhalation device to allow for the aerosolization of the dry powder formulation by dispersion into a gas stream to form an aerosol, which is captured in a chamber having an attached mouthpiece. The patient can inhale the aerosol through the mouthpiece to initiate pharmaceutical agent delivery and treatment.

In another embodiment, the formulation comprises one or more pharmaceutically acceptable suspending agents that are liquid within a conventional metered dose inhaler to form a metered dose inhaler formulation. Examples of pharmaceutically acceptable suspending agents for us in metered dose inhalers are hydrofluorocarbons (such as HFA-134a, and HFA-227) and chlorofluorocarbons (such as CFC-11, CFC-12 and CFC-114). Mixtures of the suspending agents may be used.

Treatments

The microparticles comprising iloprost are useful in a variety of inhalation-based treatments for local delivery and treatment of the lungs, or for systemic delivery via the lungs (for any treatment or prophylaxis). Relative to systemic pharmaceutical agent delivery via the oral or injectable route, local delivery of respiratory pharmaceutical agents via the pulmonary route requires smaller doses of the pharmaceutical agent and minimizes systemic toxicity because it can be delivered directly to the site of the disease.

In one embodiment, the microparticles comprising iloprost are useful in the treatment of pulmonary hypertension (PH).

In one embodiment, administration of the microparticles comprising iloprost or another pharmaceutical agent to be administered in addition to iloprost provides local or plasma concentrations sustained at approximately constant values over the intended period of release (e.g., up to 2 to 24 hours, to enable dosing once, twice, three times, four times or more than four times per day). The microparticle formulations may allow patients to take treatments less frequently, and to receive more prolonged and steadier relief.

In some embodiments, the microparticles comprising iloprost or another pharmaceutical agent to be administered in addition to iloprost comprise polymer matrices having one or more lipids or another hydrophobic or amphiphilic compound incorporated therein to modify the release kinetics. The matrices are preferably used for parenteral delivery.

In some embodiments, the microparticles have incorporated therein components or means for limiting diffusion of drug out of the microparticle.

In some embodiments, the microparticles have incorporated therein components or means for modifying the degradation kinetics of the microparticles.

In some embodiments, the microparticles are delivered to the lung.

In some embodiments, the microparticles comprising iloprost or another pharmaceutical agent to be administered in addition to iloprost comprise a lipid or other hydrophobic or amphiphilic compound (collectively referred to herein as “hydrophobic compounds”) which is integrated into a polymeric matrix for drug delivery to alter drug release kinetics. In one embodiment where the drug is water soluble, the drug is released over longer periods of time as compared to release from the polymeric matrix not incorporating the hydrophobic compound into the polymeric material. In a further embodiment where the drug has low water solubility, the drug is released over shorter periods of time as compared to release from matrix not incorporating the hydrophobic compound into the polymeric material. In contrast to methods in which a surfactant or lipid is added as an excipient, the hydrophobic compound is actually integrated into the polymeric matrix, thereby modifying the diffusion of water into the microparticle and diffusion of solubilized drug out of the matrix. The integrated hydrophobic compound also prolongs degradation of hydrolytically unstable polymers forming the matrix, further delaying release of encapsulated drug.

In some embodiments, the hydrophobic compound is incorporated into the matrix and the matrix shaped using a technique which results in integration of the hydrophobic compound into the polymeric matrix, rather than at the outer surface of the matrix. In the preferred embodiment, the matrix is formed into microparticles. The microparticles are manufactured with a diameter suitable for the intended route of administration. For example, with a diameter of between 0.5 and 8 microns for intravascular administration, a diameter of 1-100 microns for subcutaneous or intramuscular administration, and a diameter of between 0.5 and 5 mm for oral administration for delivery to the gastrointestinal tract or other lumens. A preferred size for administration to the pulmonary system is an aerodynamic diameter of between one and three microns, with an actual diameter of five microns or more. In the preferred embodiment, the polymers are synthetic biodegradable polymers. Most preferred polymers are biocompatible hydrolytically unstable polymers like polyhydroxy acids such as polylactic acid-co-glycolic acid, polylactide, polyglycolide or polyactide coglycolide, which may be conjugated to polyethylene glycol or other materials inhibiting uptake by the reticuloendothelial system (RES).

The hydrophobic compounds can be hydrophobic compounds such as some lipids, or amphiphilic compounds (which include both a hydrophilic and hydrophobic component or region). The most preferred amphiphilic compounds are phospholipids, most preferably dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (w/w polymer), most preferably between 0.1-30 (w lipid/w polymer).

Surface properties of the matrix can also be modified. For example, adhesion can be enhanced through the selection of bioadhesive polymers, which may be particularly desirable when the matrix is in the form of microparticles administered to a mucosal surface such as in intranasal, pulmonary, vaginal, or oral administration. Targeting can also be achieved by selection of the polymer or incorporation within or coupling to the polymer to ligands which specifically bind to particular tissue types or cell surface molecules. Additionally, ligands may be attached to the microparticles which effect the charge, lipophilicity or hydrophilicity of the particle.

Methods are provided for the synthesis of polymeric delivery systems consisting of polymer matrices that contain iloprost or another pharmaceutical agent to be administered in addition to iloprost. The matrices are useful in a variety of drug delivery applications and can be administered by injection, aerosol or powder, orally, or topically. A preferred route of administration of iloprost is via the pulmonary system. The incorporation of a hydrophobic and/or amphiphilic compound (referred to generally herein as “hydrophobic compound”) into the polymeric matrix modifies the period of drug release as compared with the same polymeric matrix without the incorporated hydrophobic compound, by altering the rate of diffusion of water into and out of the matrix and/or the rate of degradation of the matrix.

Reagents for Making Matrix Having Hydrophobic Compound Incorporated Therein

The matrix may be a structure including one or more materials in which a drug is dispersed, entrapped, or encapsulated. The material can be crystalline, semi-crystalline, or amorphous. The matrix can be in the form of pellets, tablets, slabs, rods, disks, hemispheres, or microparticles, or be of an undefined shape. As used herein, the term microparticle includes microspheres and microcapsules, as well as microparticles, unless otherwise specified. Microparticles may or may not be spherical in shape. Microcapsules are defined as microparticles having an outer polymer shell surrounding a core of another material, in this case, the active agent. Microspheres are generally solid polymeric spheres, which can include a honeycombed structure formed by pores through the polymer which are filled with the active agent, as described below.

Polymers

The matrix can be formed of non-biodegradable or biodegradable matrices, although biodegradable matrices are preferred, particularly for parenteral administration. Non-erodible polymers may be used for oral administration. In general, synthetic polymers are preferred due to more reproducible synthesis and degradation, although natural polymers may be used and have equivalent or even better properties, especially some of the natural biopolymers which degrade by hydrolysis, such as polyhydroxybutyrate. The polymer is selected based on the time required for in vivo stability, i.e. that time required for distribution to the site where delivery is desired, and the time desired for delivery.

Representative synthetic polymers are: poly(hydroxy acids) such as poly(lactic acid), poly(glycolic acid), and poly(lactic acid-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) jointly referred to herein as “polyacrylic acids”), poly(butyric acid), poly(valeric acid), and poly(lactide-coaprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-coaprolactone), blends and copolymers thereof.

Examples of preferred natural polymers include proteins such as albumin and prolamines, for example, zein, and polysaccharides such as alginate, cellulose and polyhydroxyalkanoates, for example, polyhydroxybutyrate. The in vivo stability of the matrix can be adjusted during the production by using polymers such as polylactide co glycolide copolymerized with polyethylene glycol (PEG). PEG if exposed on the external surface may elongate the time these materials circulate since it is hydrophilic.

Examples of preferred non-biodegradable polymers include ethylene vinyl acetate, poly(meth)acrylic acid, polyamides, copolymers and mixtures thereof.

Bioadhesive polymers of particular interest for use in targeting of mucosal surfaces, as in the gastrointestinal tract, include polyanhydrides, polyacrylic acid, poly(methyl methacrylates), poly(ethyl methacrylates), polybutylinethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Solvents

A solvent for the polymer is selected based on its biocompatibility as well as the solubility of the polymer and where appropriate, interaction with the iloprost and/or other pharmaceutical agent to be administered in addition to iloprost. For example, the ease with which the agent is dissolved in the solvent and the lack of detrimental effects of the solvent on the agent to be delivered are factors to consider in selecting the solvent. Aqueous solvents can be used to make matrices formed of water soluble polymers. Organic solvents will typically be used to dissolve hydrophobic and some hydrophilic polymers. Preferred organic solvents are volatile or have a relatively low boiling point or can be removed under vacuum and which are acceptable for administration to humans in trace amounts, such as methylene chloride. Other solvents, such as ethyl acetate, ethanol, methanol, dimethyl formamide (DMF), acetone, acetonitrile, tetrahydrofuran (THF), acetic acid, dimethyle sulfoxide (DMSO) and chloroform, and combinations thereof, also may be utilized. Preferred solvents are those rated as class 3 residual solvents by the Food and Drug Administration, as published in the Federal Register vol. 62, number 85, pp. 24301-24309 (May 1997).

In general, the polymer is dissolved in the solvent to form a polymer solution having a concentration of between 0.1 and 60% weight to volume (w/v), more preferably between 0.25 and 30%. The polymer solution is then processed as described below to yield a polymer matrix having hydrophobic components incorporated therein.

Hydrophobic and Amphiphilic Compounds

In general, compounds which are hydrophobic or amphiphilic (i.e., including both a hydrophilic and a hydrophobic component or region) can be used to modify penetration and/or uptake of water by the matrix, thereby modifying the rate of diffusion of drug out of the matrix, and in the case of hydrolytically unstable materials, alter degradation and thereby release of drug from the matrix.

Lipids which may be used include, but are not limited to, the following classes of lipids: fatty acids and derivatives, mono-, di and triglycerides, phospholipids, sphingolipids, cholesterol and steroid derivatives, terpenes and vitamins. Fatty acids and derivatives thereof may include, but are not limited to, saturated and unsaturated fatty acids, odd and even number fatty acids, cis and trans isomers, and fatty acid derivatives including alcohols, esters, anhydrides, hydroxy fatty acids and prostaglandins. Saturated and unsaturated fatty acids that may be used include, but are not limited to, molecules that have between 12 carbon atoms and 22 carbon atoms in either linear or branched form. Examples of saturated fatty acids that may be used include, but are not limited to, lauric, myristic, palmitic, and stearic acids. Examples of unsaturated fatty acids that may be used include, but are not limited to, lauric, physeteric, myristoleic, palmitoleic, petroselinic, and oleic acids. Examples of branched fatty acids that may be used include, but are not limited to, isolauric, isomyristic, isopalmitic, and isostearic acids and isoprenoids. Fatty acid derivatives include 12-(((7′-diethylaminocoumarin-3yl)carbonyl)methylamino)-octadecanoic acid; N-[12-(((7′diethylaminocoumarin-3-yl) carbonyl)methyl-amino) octadecanoyl]-2-aminopalmitic acid, N succinyl-dioleoylphosphatidylethanol amine and palmitoyl-homocysteine; and/or combinations thereof. Mono, di and triglycerides or derivatives thereof that may be used include, but are not limited to, molecules that have fatty acids or mixtures of fatty acids between 6 and 24 carbon atoms, digalactosyldiglyceride, 1,2-dioleoyl-sn-glycerol; 1,2-cdipalmitoyl-sn-3 succinylglycerol; and 1,3-dipalmitoyl-2-succinylglycerol.

Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and .beta.-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) may also be used.

Sphingolipids which may be used include ceramides, sphingomyelins, cerebrosides, gangliosides, sulfatides and lysosulfatides. Examples of Sphinglolipids include, but are not limited to, the gangliosides GM1 and GM2.

Steroids which may be used include, but are not limited to, cholesterol, cholesterol sulfate, cholesterol hemisuccinate, 6-(5-cholesterol 3.beta.-yloxy) hexyl-6-amino-6-deoxy-1-thio-.alpha.-D-galactopyranoside, 6-(5-cholesten-3.beta.-tloxy)hexyl-6-amino-6-deoxy]-1-thio-.alpha.-D mannopyranoside and cholesteryl)4′-trimethyl 35 ammonio)butanoate.

Additional lipid compounds which may be used include tocopherol and derivatives, and oils and derivatized oils such as stearlyamine.

A variety of cationic lipids such as DOTMA, N-[1-(2,3-dioleoyloxy)propyl-N,N,N-trimethylammonium chloride; DOTAP, 1,2-dioleoyloxy-3-trimethylammonio) propane; and DOTB, 1,2-dioleoyl-3-(4′-trimethyl-ammonio).butanoyl-sn glycerol may be used.

The most preferred lipids are phospholipids, preferably DPPC, DAPC, DSPC, DTPC, DBPC, DLPC and most preferably DPPC, DAPC and DBPC.

Other preferred hydrophobic compounds include amino acids such as tryptophane, tyrosine, isoleucine, leucine, and valine, aromatic compounds such as an alkyl paraben, for example, methyl paraben, and benzoic acid.

The content of hydrophobic compound ranges from 0.1-60 wt % (weight hydrophobic compound/weight polymer); most preferably between 0.1-30 wt % (weight hydrophobic compound/weight polymer).

Targeting

Microparticles can be targeted specifically or non-specifically through the selection of the polymer forming the microparticle, the size of the microparticle, and/or incorporation or attachment of a ligand to the microparticles. For example, biologically active molecules, or molecules affecting the charge, lipophilicity or hydrophilicity of the particle, may be attached to the surface of the microparticle. Additionally, molecules may be attached to the microparticles which minimize tissue adhesion, or which facilitate specific targeting of the microparticles in vivo. Representative targeting molecules include antibodies, lectins, and other molecules which are specifically bound by receptors on the surfaces of cells of a particular type.

Inhibition of Uptake by the RES

Uptake and removal of the microparticles can be minimized through the selection of the polymer and/or incorporation or coupling of molecules which minimize adhesion or uptake. For example, tissue adhesion by the microparticle can be minimized by covalently binding poly(alkylene glycol) moieties to the surface of the microparticle. The surface poly(alkylene glycol) moieties have a high affinity for water that reduces protein adsorption onto the surface of the particle. The recognition and uptake of the microparticle by the reticulo-endothelial system (RES) is therefore reduced.

In one method, the terminal hydroxyl group of the poly(alkylene glycol) is covalently attached to biologically active molecules, or molecules affecting the charge, lipophilicity or hydrophilicity of the particle, onto the surface of the microparticle. Methods available in the art can be used to attach any of a wide range of ligands to the microparticles to enhance the delivery properties, the stability or other properties of the microparticles in vivo.

Methods for Manufacture of Matrix

In the most preferred embodiment, microparticles are produced by spray drying. Techniques which can be used to make other types of matrices, as well as microparticles, include melt extrusion, compression molding, fluid bed drying, solvent extraction, hot melt encapsulation, and solvent evaporation, as discussed below. Preferably, the hydrophobic compound may be dissolved or melted with the polymer or dispersed as a solid or a liquid in a solution of the polymer, prior to forming the matrix. As a result, the hydrophobic (or amphiphilic) compound is mixed throughout the matrix, in a relatively uniform manner, not just on the surface of the finished matrix. The iloprost and/or other pharmaceutical agent to be administered in addition to iloprost can be incorporated into the matrix as solid particles, as a liquid or liquid droplets, or by dissolving the agent in the polymer solvent.

Solvent Evaporation

In this method the polymer and hydrophobic compound are dissolved in a volatile organic solvent such as methylene chloride. A pore forming agent as a solid or as a liquid may be added to the solution. The active agent can be added as either a solid or in solution to the polymer solution. The mixture is sonicated or homogenized and the resulting dispersion or emulsion is added to an aqueous solution that may contain a surface active agent such as TWEEN™ 20, TWEEN™80, PEG or poly(vinyl alcohol) and homogenized to form an emulsion. The resulting emulsion is stirred until most of the organic solvent evaporates, leaving microparticles. Several different polymer concentrations can be used (0.05-0.60 g/ml). Microparticles with different sizes (1-1000 microns) and morphologies can be obtained by this method. This method is particularly useful for relatively stable polymers like polyesters.

Solvent evaporation is described by E. Mathiowitz, et al., J. Scanning Microscopy, 4, 329 (1990); L. R. Beck, et al., Ferlil. Steril., 31, 545 (1979); and S. Benita, et al., J. Pharm. Sci., 73, 1721 (1984), the teachings of which are incorporated herein.

Particularly hydrolytically unstable polymers, such as polyanhydrides, may degrade during the fabrication process due to the presence of water. For these polymers, the following two methods, which are performed in completely organic solvents, are more useful.

Hot Melt Microencapsulation

In this method, the polymer and the hydrophobic compound are first melted and then mixed with the solid or liquid active agent. A pore forming agent as a solid or in solution may be added to the solution. The mixture is suspended in a non-miscible solvent (like silicon oil), and, while stirring continuously, heated to 5 degrees C. above the melting point of the polymer. Once the emulsion is stabilized, it is cooled until the polymer particles solidify. The resulting microparticles are washed by decantation with a polymer non-solvent such as petroleum ether to give a free-flowing powder. Microparticles with sizes between one to 1000 microns can be obtained with this method. The external surfaces of particles prepared with this technique are usually smooth and dense. This procedure is used to prepare microparticles made of polyesters and polyanhydrides. However, this method is limited to polymers with molecular weights between 1000-50,000.

Hot-melt microencapsulation is described by E. Mathiowitz, et al., Reactive Polymers, 6, 275 (1987), the teachings of which are incorporated herein. Preferred polyanhydrides include polyanhydrides made of bis-carboxyphenoxypropane and sebacic acid with molar ratio of 20:80 (P(CPP-SA) 20:80) (MW 20,000) and poly(fumaricosebacic) (20:80) (MW 15,000) microparticles.

Solvent Removal

This technique was primarily designed for polyanhydrides. In this method, the solid or liquid active agent is dispersed or dissolved in a solution of the selected polymer and hydrophobic compound in a volatile organic solvent like methylene chloride. This mixture is suspended by stirring in an organic oil (such as silicon oil) to form an emulsion. Unlike solvent evaporation, this method can be used to make microparticles from polymers with high melting points and different molecular weights. The external morphology of particles produced with this technique is highly dependent on the type of polymer used.

Spray Drying of Microparticles

Microparticles can be produced by spray drying by dissolving a biocompatible polymer and hydrophobic compound in an appropriate solvent, dispersing a solid or liquid active agent into the polymer solution, and then spray drying the polymer solution, to form microparticles. For example, a solution of a polymer and an active agent refers may be atomized to form a fine mist and dried by direct contact with hot carrier gases. Using spray drying apparatus available in the art, the polymer solution may be delivered through the inlet port of the spray drier, passed through a tube within the drier and then atomized through the outlet port. The temperature may be varied depending on the gas or polymer used. The temperature of the inlet and outlet ports can be controlled to produce the desired products.

The size of the particulates of polymer solution is a function of the nozzle used to spray the polymer solution, nozzle pressure, the flow rate, the polymer used, the polymer concentration, the type of solvent and the temperature of spraying (both inlet and outlet temperature) and the molecular weight. Generally, the higher the molecular weight, the larger the particle size, assuming the concentration is the same. Typical process parameters for spray drying are as follows: polymer concentration=0.005-0.20 g/ml, inlet temperature=20-1000° C., outlet temperature=10-300° C., polymer flow rate=5-2000 m/min., and nozzle diameter between one and ten microns can be obtained with a morphology which depends on the selection of polymer, concentration, molecular weight and spray flow.

If the active agent is a solid, the agent may be encapsulated as solid particles which are added to the polymer solution prior to spraying, or the agent can be dissolved in an aqueous solution which then is emulsified with the polymer solution prior to spraying, or the solid may be cosolubilized together with the polymer in an appropriate solvent prior to spraying.

Hydrogel Microparticles

Microparticles made of gel-type polymers, such as polyphosphazene or polymethylmethacrylate, are produced by dissolving the polymer in an aqueous solution, suspending if desired a pore forming agent and suspending a hydrophobic compound in the mixture, homogenizing the mixture, and extruding the material through a microdroplet forming device, producing microdroplets which fall into a hardening bath consisting of an oppositely charged ion or polyelectrolyte solution, that is slowly stirred. The advantage of these systems is the ability to further modify the surface of the microparticles by coating them with polycationic polymers, like polylysine after fabrication. Microparticle particles are controlled by using various size extruders.

Additives to Facilitate Matrix Formation

A variety of surfactants may be added to the continuous phase as emulsifiers if one is used during the production of the matrices. Exemplary emulsifiers or surfactants which may be used (0.1-5% by weight) include most physiologically acceptable emulsifiers. Examples include natural and synthetic forms of bile salts or bile acids, both conjugated with amino acids and unconjugated such as taurodeoxycholate, and cholic acid. In contrast to the methods described herein, these surfactant will coat the microparticle and will facilitate dispersion for administration.

Pore Forming Agents

Pore forming agents can be included in an amount of between 0.01% and 90% weight to volume, to increase matrix porosity and pore formation during the production of the matrices. The pore forming agent can be added as solid particles to the polymer solution or melted polymer or added as an aqueous solution which is emulsified with the polymer solution or is co-dissolved in the polymer solution. For example, in spray drying, solvent evaporation, solvent removal, hot melt encapsulation, a pore forming agent such as a volatile salt, for example, ammonium bicarbonate, ammonium acetate, ammonium chloride or ammonium benzoate or other lyophilizable salt, is first dissolved in water. The solution containing the pore forming agent is then emulsified with the polymer solution to create droplets of the pore forming agent in the polymer. This emulsion is then spray dried or taken through a solvent evaporation/extraction process. After the polymer is precipitated, the hardened microparticles can be frozen and lyophilized to remove any pore forming agents not removed during the microencapsulation process.

Methods for Administration of Drug Delivery Systems

The matrix is administered orally, topically, to a mucosal surface (i.e., nasal, pulmonary, vaginal, rectal), or by implantation or injection, depending on the form of the matrix and the agent to be delivered. Preferably, the microparticles containing iloprost are administered to the pulmonary system. Useful pharmaceutically acceptable carriers include saline containing glycerol and TWEEN™20 and isotonic mannitol containing TWEEN™ 20. The matrix can also be in the form of powders, tablets, in capsules, or in a topical formulation such as an ointment, gel or lotion.

Microparticles can be administered as a powder, or formulated in tablets or capsules, suspended in a solution or in a gel (ointment, lotion, hydrogel). As noted above, the size of the microparticles is determined by the method of administration. In the preferred embodiment, the microparticles are manufactured with a diameter of between 0.5 and 8 microns for intravascular administration, a diameter of 1-100 microns for subcutaneous or intramuscular administration, and a diameter of between 0.5 and 5 mm for oral administration for delivery to the gastrointestinal tract or other lumens, or application to other mucosal surfaces (rectal, vaginal, oral, nasal). A preferred size for administration to the pulmonary system is an aerodynamic diameter of between one and three microns, with an actual diameter of five microns or more, as described in U.S. Pat. No. 5,855,913, which issued on Jan. 5, 1999, to Edwards, et al. Particle size analysis can be performed on a Coulter counter, by light microscopy, scanning electron microscopy, or transmittance electron microscopy.

In the preferred embodiment, microparticles are combined with a pharmaceutically acceptable carrier such as phosphate buffered saline or saline or mannitol, then an effective amount administered to a patient using an appropriate route, such as nasally, via a blood vessel (i.v.), subcutaneously, intramuscularly (IM) or orally. Microparticles containing an active agent may be used for delivery to the vascular system, as well as delivery to the liver and renal systems, in cardiology applications, and in treating tumor masses and tissues. Preferably, microparticles comprising iloprost are administered to the pulmonary system. In some embodiments, for administration to the pulmonary system, the microparticles can be combined with pharmaceutically acceptable bulking agents and administered as a dry powder. Pharmaceutically acceptable bulking agents include sugars such as mannitol, sucrose, lactose, fructose and trehalose. The microparticles also can be linked with ligands that minimize tissue adhesion or that target the microparticles to specific regions of the body in vivo as described above.

The methods and compositions described above will be further understood with reference to the following non-limiting examples.

Some embodiments of the present invention relate to microparticles comprising iloprost or comprising another pharmaceutical agent to be administered in addition to iloprost, comprise an amino acid or salt thereof. In some embodiments, the microparticles comprising iloprost or another pharmaceutical agent to be administered in addition to iloprost have a tap density of less than about 0.4 g/cm³. Preferably, the microparticles have a tap density of less than about 0.4 g/cm³ include an amino acid or a salt thereof.

In a preferred embodiment the amino acid in the microparticles is hydrophobic. Suitable hydrophobic amino acids include naturally occurring and non-naturally occurring hydrophobic amino acids. Non-naturally occurring amino acids include, for example, beta-amino acids, Both D, L and racemic configurations of hydrophobic amino acids can be employed. Suitable hydrophobic amino acids can also include amino acid analogs. As used herein, an amino acid analog includes the D or L configuration of an amino acid having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C₁-C₈ hydrocarbons which are completely saturated, which contain one or two heteroatoms such as nitrogen, oxygen or sulfur and/or which contain one or more units of unsaturation. Aromatic groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

Suitable substituents on an aliphatic, aromatic or benzyl group include —OH, halogen (—Br, —Cl, —I and —F) —O(aliphatic, substituted aliphatic, benzyl, substituted benzyl, aryl or substituted aryl group), —CN, —NO₂, —COOH, —NH₂, —NH(aliphatic group, substituted aliphatic, benzyl, substituted benzyl, aryl or substituted aryl group), —N(aliphatic group, substituted aliphatic, benzyl, substituted benzyl, aryl or substituted aryl group)₂, —COO(aliphatic group, substituted aliphatic, benzyl, substituted benzyl, aryl or substituted aryl group), —CONH₂, —CONH(aliphatic, substituted aliphatic group, benzyl, substituted benzyl, aryl or substituted aryl group)), —SH, —S(aliphatic, substituted aliphatic, benzyl, substituted benzyl, aromatic or substituted aromatic group) and —NH—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Modifying an amino acid substituent can increase, for example, the lypophilicity or hydrophobicity of natural amino acids which are hydrophillic.

A number of the suitable amino acids, amino acids analogs and salts thereof can be obtained commercially. Others can be synthesized by methods known in the art. Synthetic techniques are described, for example, in Green and Wuts, “Protecting Groups in Organic Synthesis”, John Wiley and Sons, Chapters 5 and 7, 1991.

Hydrophobicity is generally defined with respect to the partition of an amino acid between a nonpolar solvent and water. Hydrophobic amino acids are those acids which show a preference for the nonpolar solvent. Relative hydrophobicity of amino acids can be expressed on a hydrophobicity scale on which glycine has the value 0.5. On such a scale, amino acids which have a preference for water have values below 0.5 and those that have a preference for nonpolar solvents have a value above 0.5. As used herein, the term hydrophobic amino acid refers to an amino acid that, on the hydrophobicity scale has a value greater or equal to 0.5, in other words, has a tendency to partition in the nonpolar acid which is at least equal to that of glycine.

Examples of amino acids which can be employed include, but are not limited to: glycine, proline, alanine, cysteine, methionine, valine, leucine, tyrosine, isoleucine, phenylalanine, tryptophan. Preferred hydrophobic amino acids include leucine, isoleucine, alanine, valine, phenylalanine and glycine. Combinations of hydrophobic amino acids can also be employed. Furthermore, combinations of hydrophobic and hydrophilic (preferentially partitioning in water) amino acids, where the overall combination is hydrophobic, can also be employed.

In a preferred embodiment of the invention, the amino acid is insoluble in the solvent system employed, such as, for example, in a 70:30 (vol/vol) ethanol:water co-solvent. The amino acid can be present in the microparticles of in an amount of at least about 10 weight %. Preferably, the amino acid can be present in the microparticles in an amount ranging from about 20 to about 80 weight %. The salt of a hydrophobic amino acid can be present in the microparticles of the invention in an amount of at least about 10 weight %. Preferably, the amino acid salt is present in the microparticles in an amount ranging from about 20 to about 80 weight %.

In some embodiments, the microparticles comprising iloprost or another pharmaceutical agent to be administered in addition to iloprost can also be precursors to tablet formulations.

In some embodiments, the iloprost and/or other pharmaceutical agent to be administered in addition to iloprost can be present in the spray-dried microparticles in an amount ranging from less than about 1 weight % to about 90 weight %.

In another embodiment of the invention, the microparticles include a phospholipid, also referred to herein as phosphoglyceride. In one embodiment, the phospholipid is endogenous to the lung. In one embodiment the phospholipid includes, among others, phosphatidylcholines, phosphatidylethanolamines, phosphatidylglycerols, phosphatidylserines, phosphatidylinositols and combinations thereof. Specific examples of phospholipids include but are not limited to phosphatidylcholines dipalmitoyl phosphatidylcholine (DPPC), dipalmitoyl phosphatidylethanolamine (DPPE), distearoyl phosphatidylcholine (DSPC), dipalmitoyl phosphatidyl glycerol (DPPG) or any combination thereof.

In some embodiments, the phospholipid can be present in the microparticles in an amount ranging from about 0 to about 90 weight %. In other embodiments, it can be present in the microparticles in an amount ranging from about 10 to about 60 weight %.

In still another embodiment the microparticles include a surfactant such as, but not limited to the surfactants and phospholipids described above. For example, the surfactant can be hexadecanol; fatty alcohols such as polyethylene glycol (PEG); polyoxyethylene-9-lauryl ether; a surface active fatty acid, such as palmitic acid or oleic acid; glycocholate; surfactin; a poloxomer; a sorbitan fatty acid ester such as sorbitan trioleate (Span 85); tyloxapol can also be employed.

In some embodiments the surfactant may be any agent which preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to microparticles, they tend to present moieties to the external environment that do not attract similarly-coated microparticles, thus reducing microparticle agglomeration. Surfactants may also promote absorption of a therapeutic or diagnostic agent and increase bioavailability of the agent.

The surfactant can be present in the microparticles in an amount ranging from about 0 to about 90 weight %. Preferably, it can be present in the microparticles in an amount ranging from about 10 to about 60 weight %.

In some embodiments, the microparticles include iloprost and/or another pharmaceutical agent to be administered in addition to iloprost, a hydrophobic amino acid or a salt thereof, and a phospholipid.

In one embodiment of the invention, the phospholipid or combination or phospholipids present in the microparticles can have a therapeutic, prophylactic or diagnostic role. For example, the microparticles of the invention can be used to deliver surfactants to the lung of a patient.

In some embodiments, the microparticles provide controlled or sustained release of the iloprost and/or another pharmaceutical agent to be administered in addition to the iloprost. In some embodiments, the spray-dried microparticles can include a biocompatible, and preferably biodegradable polymer, copolymer, or blend. Preferred polymers are those which are capable of forming aerodynamically light microparticles having a tap density less than about 0.4 g/cm³, a mean diameter between about 5 micrometers and about 30 micrometers and an aerodynamic diameter between approximately one and five microns, preferably between about one and about three microns. The polymers can be tailored to optimize different characteristics of the particle including: i) interactions between the iloprost or other pharmaceutical agent to be administered in addition to iloprost and the polymer to provide stabilization of the iloprost and/or other pharmaceutical agent and retention of activity upon delivery; ii) rate of polymer degradation and, thereby, rate of drug release profiles; iii) surface characteristics and targeting capabilities via chemical modification; and iv) particle porosity.

Surface eroding polymers such as polyanhydrides can be used to form the microparticles. For example, polyanhydrides such as poly[(p-carboxyphenoxy)-hexane anhydride] (PCPH) may be used. Suitable biodegradable polyanhydrides are described in U.S. Pat. No. 4,857,311.

In another embodiment, bulk eroding polymers such as those based on polyesters including poly(hydroxy acids) can be used. For example, polyglycolic acid (PGA), polylactic acid (PLA), or copolymers thereof may be used to form the microparticles. The polyester may also have a charged or functionalizable group, such as an amino acid. In a preferred embodiment, microparticles with controlled release properties can be formed of poly(D,L-lactic acid) and/or poly(D,L-lactic-co-glycolic acid) (“PLGA”) which incorporate a phospholipid such as DPPC.

Still other polymers include but are not limited to polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, and polyvinyl esters, polymers of acrylic and methacrylic acids, celluloses and other polysaccharides, and peptides or proteins, or copolymers or blends thereof. Polymers may be selected with or modified to have the appropriate stability and degradation rates in vivo for different controlled drug delivery applications.

In one embodiment, the microparticles include functionalized polyester graft copolymers, as described in Hrkach et al., Macromolecules, 28: 4736-4739 (1995); and Hrkach et al., “Poly(L-Lactic acid-co-amino acid) Graft Copolymers: A Class of Functional Biodegradable Biomaterials” in Hydrogels and Biodegradable Polymers for Bioapplications, ACS Symposium Series No. 627, Raphael M. Ottenbrite et al., Eds., American Chemical Society, Chapter 8, pp. 93-101, 1996.

Materials other than biodegradable polymers can be included in the spray-dried microparticles of the invention. Suitable materials include various non-biodegradable polymers and various excipients. Examples of excipients include, but are not limited to: a sugar, such as lactose, polysaccharides, cyclodextrins and/or a surfactant.

The microparticles of the invention can be employed in compositions suitable for drug delivery to the pulmonary system. For example, such compositions can include the microparticles and a pharmaceutically acceptable carrier for administration to a patient, preferably for administration via inhalation. The microparticles can be co-delivered, for example, with larger carrier particles, not carrying a therapeutic agent, having, for example, a mean diameter ranging between about 50 micrometers and about 100 micrometers.

The microparticles of the invention preferably have a tap density less than about 0.4 g/cm³. As used herein, the phrase “aerodynamically light microparticles” refers to microparticles having a tap density less than about 0.4 g/cm³. The tap density of microparticles of a dry powder can be obtained using a GeoPyc™ instrument (Micrometrics Instrument Corp., Norcross, Ga. 30093). A Dual Platform Microprocessor Controlled Tap Density Tester (Vankel, N.C.) can also be used. Tap density is a standard measure of the envelope mass density. The envelope mass density of an isotropic particle is defined as the mass of the particle divided by the minimum sphere envelope volume within which it can be enclosed. Features which can contribute to low tap density include irregular surface texture and porous structure.

The preferred median diameter for aerodynamically light microparticles for inhalation therapy is at least about 5 microns (μm), for example between about 5 and about 30 micrometers. Terms such as median diameter, mass median diameter (MMD), mass median geometric diameter (MMGD) and mass median envelope diameter (MMED) are herein used interchangeably. The term diameter, in contrast with the term “aerodynamic diameter”, refers herein to mass or geometric diameter. The terms “aerodynamic diameter” and “mass median aerodynamic diameter” (MMAD) are used herein interchangeably. In one embodiment of the invention, the mass median aerodynamic diameter is between about 1 micrometer and about 5 micrometers. In another embodiment of the invention, the mass median aerodynamic diameter is between about 1 micrometers and about 3 micrometers. In a further embodiment, the mass median aerodynamic diameter is between about 3 micrometers and about 5 micrometers.

The mass median diameter of the spray-dried microparticles can be measured using an electrical zone sensing instrument such as a Multisizer IIe, (Coulter Corp., Miami, Fla.), or a laser diffraction instrument (for example Helos, manufactured by Sympatec, Princeton, N.J.). The diameter of microparticles in a sample will range depending upon factors such as microparticle composition and methods of synthesis. The distribution of size of microparticles in a sample can be selected to permit optimal deposition within targeted sites within the respiratory tract.

Process conditions as well as efficiency of inhaler, in particular with respect to dispersibility, can contribute to the size of microparticles that can be delivered to the pulmonary system.

Aerodynamically light microparticles may be fabricated or separated, for example by filtration or centrifugation, to provide a microparticle sample with a preselected size distribution. For example, greater than about 30%, 50%, 70%, or 80% of the microparticles in a sample can have a diameter within a selected range of at least about 5 micrometers. The selected range within which a certain percentage of the microparticles must fall may be for example, between about 5 and about 30 micrometers, or optimally between about 5 and about 15 micrometers. In one preferred embodiment, at least a portion of the microparticles have a diameter between about 9 and about 11 micrometers. Optionally, the microparticle sample also can be fabricated wherein at least about 90%, or optionally about 95% or about 99%, have a diameter within the selected range. The presence of the higher proportion of the aerodynamically light, larger diameter microparticles in the particle sample enhances the delivery of therapeutic or diagnostic agents incorporated therein to the deep lung. Large diameter microparticles generally mean microparticles having a median geometric diameter of at least about 5 micrometers.

Aerodynamically light microparticles with a tap density less than about 0.4 g/cm³, median diameters of at least about 5 micrometers, and an aerodynamic diameter of between about 1 and about 5 micrometers, preferably between about 1 and about 3 micrometers, are more capable of escaping inertial and gravitational deposition in the oropharyngeal region, and are targeted to the airways or the deep lung. The use of larger, more porous microparticles is advantageous since they are able to aerosolize more efficiently than smaller, denser aerosol microparticles such as those currently used for inhalation therapies.

In comparison to smaller, relatively denser microparticles the larger aerodynamically light microparticles, preferably having a median diameter of at least about 5 micrometers, also can potentially more successfully avoid phagocytic engulfment by alveolar macrophages and clearance from the lungs, due to size exclusion of the microparticles from the phagocytes' cytosolic space. Phagocytosis of microparticles by alveolar macrophages diminishes precipitously as particle diameter increases beyond about 3 μm. Kawaguchi, H., et al., Biomaterials 7: 61-66 (1986); Krenis, L. J. and Strauss, B., Proc. Soc. Exp. Med., 107: 748-750 (1961); and Rudt, S. and Muller, R. H., J. Contr. Rel., 22: 263-272 (1992). For microparticles of statistically isotropic shape, such as spheres with rough surfaces, the particle envelope volume is approximately equivalent to the volume of cytosolic space required within a macrophage for complete particle phagocytosis.

Aerodynamically light microparticles thus are capable of a longer term release of an encapsulated agent in the lungs. Following inhalation, aerodynamically light biodegradable microparticles can deposit in the lungs, and subsequently undergo slow degradation and drug release, without the majority of the microparticles being phagocytosed by alveolar macrophages. The drug can be delivered relatively slowly into the alveolar fluid, and at a controlled rate into the blood stream, minimizing possible toxic responses of exposed cells to an excessively high concentration of the drug. The aerodynamically light microparticles thus are highly suitable for inhalation therapies, particularly in controlled release applications.

The microparticles may be fabricated with the appropriate material, surface roughness, diameter and tap density for localized delivery to selected regions of the respiratory tract such as the deep lung or upper or central airways. For example, higher density or larger microparticles may be used for upper airway delivery, or a mixture of varying sized microparticles in a sample, provided with the same or different therapeutic agent may be administered to target different regions of the lung in one administration. Microparticles having an aerodynamic diameter ranging from about 3 to about 5 micrometers are preferred for delivery to the central and upper airways. Microparticles having and aerodynamic diameter ranging from about 1 to about 3 micrometers are preferred for delivery to the deep lung.

Inertial impaction and gravitational settling of aerosols are predominant deposition mechanisms in the airways and acini of the lungs during normal breathing conditions. Edwards, D. A., J. Aerosol Sci., 26: 293-317 (1995). The importance of both deposition mechanisms increases in proportion to the mass of aerosols and not to particle (or envelope) volume. Since the site of aerosol deposition in the lungs is determined by the mass of the aerosol (at least for microparticles of mean aerodynamic diameter greater than approximately 1 μm), diminishing the tap density by increasing particle surface irregularities and particle porosity permits the delivery of larger particle envelope volumes into the lungs, all other physical parameters being equal.

The low tap density microparticles have a small aerodynamic diameter in comparison to the actual envelope sphere diameter. The aerodynamic diameter, daer, is related to the envelope sphere diameter, d (Gonda, I., “Physico-chemical principles in aerosol delivery,” in Topics in Pharmaceutical Sciences 1991 (eds. D. J. A. Crommelin and K. K. Midha), pp. 95-117, Stuttgart: Medpharm Scientific Publishers, 1992)), by the formula:
dₐₑᵣ√ρ (EQ. 10)
where the envelope mass p is in units of g/cm³. Maximal deposition of monodispersed aerosol microparticles in the alveolar region of the human lung (˜60%) occurs for an aerodynamic diameter of approximately dₐₑᵣ=3 μm. Heyder, J. et al., J. Aerosol Sci., 17: 811-825 (1986). Due to their small envelope mass density, the actual diameter d of aerodynamically light microparticles comprising a monodisperse inhaled powder that will exhibit maximum deep-lung deposition is:
d=3/√ρ μm (where p<1 g/cm³) (EQ. 11)
where d is always greater than 3 μm. For example, aerodynamically light microparticles that display an envelope mass density, ρ=0.1 g/cm³, will exhibit a maximum deposition for microparticles having envelope diameters as large as 9.5 μm. The increased particle size diminishes interparticle adhesion forces. Visser, J., Powder Technology, 58: 1-10. Thus, large particle size increases efficiency of aerosolization to the deep lung for microparticles of low envelope mass density, in addition to contributing to lower phagocytic losses.

In one embodiment of the invention, the spray-dried microparticles have a tap density less than about 0.4 g/cm³ and a median diameter between about 5 micrometers and about 30 micrometers, which in combination yield an aerodynamic diameter of between about 1 and about 5 micrometers, and for delivery to the deep lung, preferably between about 1 and about 3 micrometers. The aerodyanamic diameter is calculated to provide for maximum deposition within the lungs, previously achieved by the use of very small microparticles of less than about five microns in diameter, preferably between about one and about three microns, which are then subject to phagocytosis. Selection of microparticles which have a larger diameter, but which are sufficiently light (hence the characterization “aerodynamically light”), results in an equivalent delivery to the lungs, but the larger size microparticles are not phagocytosed. Improved delivery can be obtained by using microparticles with a rough or uneven surface relative to those with a smooth surface.

In another embodiment of the invention, the microparticles have a mass density of less than about 0.4 g/cm³ and a mean diameter of between about 5 μm and about 30 μm. Mass density and the relationship between mass density, mean diameter and aerodynamic diameter are discussed in U.S. application Ser. No. 08/655,570, filed on May 24, 1996, which is incorporated herein by reference in its entirety. In a preferred embodiment, the aerodynamic diameter of microparticles having a mass density less than about 0.4 g/cm³ and a mean diameter of between about 5 micrometers and about 30 micrometers is between about 1 micrometer and about 5 micrometers.

The invention also relates to methods of preparing microparticles having a tap density less than about 0.4 g/cm³. In one embodiment, the method includes forming a mixture including iloprost and/or another pharmaceutical agent to be administered in addition to iloprost, or a combination thereof, and an amino acid or a salt thereof. The therapeutic, prophylactic or diagnostic agents which can be employed include but are not limited to those described above. The amino acids or salts thereof, include but are not limited to those described before.

In a preferred embodiment, the mixture includes a surfactant, such as, for example, the surfactants described above. In another preferred embodiment, the mixture includes a phospholipid, such as, for example the phospholipids described above. An organic solvent or an aqueous-organic solvent can be employed to form the mixture.

Suitable organic solvents that can be employed include but are not limited to alcohols such as, for example, ethanol, methanol, propanol, isopropanol, butanols, and others. Other organic solvents include but are not limited to perfluorocarbons, dichloromethane, chloroform, ether, ethyl acetate, methyl tert-butyl ether and others.

Co-solvents that can be employed include an aqueous solvent and an organic solvent, such as, but not limited to, the organic solvents as described above. Aqueous solvents include water and buffered solutions. In one embodiment, an ethanol water solvent is preferred with the ethanol:water ratio ranging from about 50:50 to about 90:10 ethanol:water.

The mixture can have a neutral, acidic or alkaline pH. Optionally, a pH buffer can be added to the solvent or co-solvent or to the formed mixture. Preferably, the pH can range from about 3 to about 10.

The mixture is spray-dried. Suitable spray-drying techniques are described, for example, by K. Masters in “Spray Drying Handbook”, John Wiley & Sons, New York, 1984. Generally, during spray-drying, heat from a hot gas such as heated air or nitrogen is used to evaporate the solvent from droplets formed by atomizing a continuous liquid feed.

In a preferred embodiment, a rotary atomizer is employed. An examples of suitable spray driers using rotary atomization includes the Mobile Minor spray drier, manufactured by Niro, Denmark. The hot gas can be, for example, air, nitrogen or argon.

Without being held to any particular theory, it is believed that due to their hydrophobicity and low water solubility, hydrophobic amino acids facilitate the formation of a shell during the drying process when an ethanol:water co-solvent is employed. It is also believed that the amino acids may alter the phase behavior of the phospholipids in such a way as to facilitate the formation of a shell during the drying process.

The microparticles of the invention can be used for delivery of iloprost and/or another pharmaceutical agent to be administered in addition to iloprost to the pulmonary system. They can be used to provide controlled systemic or local delivery of the iloprost and/or other therapeutic agent to be administered in addition to iloprost to the respiratory tract via aerosolization. Administration of the microparticles to the lung by aerosolization permits deep lung delivery of relatively large diameter therapeutic aerosols, for example, greater than about 5 μm in median diameter. The microparticles can be fabricated with a rough surface texture to reduce particle agglomeration and improve flowability of the powder. The spray-dried microparticles have improved aerosolization properties. The spray-dried particle can be fabricated with features which enhance aerosolization via dry powder inhaler devices, and lead to lower deposition in the mouth, throat and inhaler device.

The microparticles may be administered alone or in any appropriate pharmaceutically acceptable carrier, such as a liquid, for example saline, or a powder, for administration to the respiratory system. They can be co-delivered with larger carrier microparticles, not including a therapeutic agent, the latter possessing mass median diameters for example in the range between about 50 μm and about 100 micrometers.

Aerosol dosage, formulations and delivery systems may be selected for a particular therapeutic application, as described, for example, in Gonda, I. “Aerosols for delivery of therapeutic and diagnostic agents to the respiratory tract,” in Critical Reviews in Therapeutic Drug Carrier Systems, 6: 273-313, 1990; and in Moren, “Aerosol dosage forms and formulations,” in: Aerosols in Medicine. Principles. Diagnosis and Therapy, Moren, et al., Eds, Elsevier, Amsterdam, 1985.

The use of biodegradable polymers permits controlled release in the lungs and long-time local action or systemic bioavailability. Denaturation of macromolecular drugs can be minimized during aerosolization since macromolecules can be contained and protected within a polymeric shell. Coencapsulation of peptides with peptidase-inhibitors can minimize peptide enzymatic degradation. Pulmonary delivery advantageously can reduce or eliminate the need for injection.

The invention is also related to a method for delivery of iloprost and/or another pharmaceutical agent to be administered in addition to iloprost to the pulmonary system. The method comprises administering to the respiratory tract of a patient in need of treatment, prophylaxis or diagnosis an effective amount of microparticles comprising iloprost and a hydrophobic amino acid. In a preferred embodiment, the microparticles include a phospholipid. As used herein, the term “effective amount” means the amount needed to achieve the desired effect or efficacy. In some embodiments, the patient may be suffering from pulmonary hypertension.

Porous or aerodynamically light microparticles, having a geometric size (or mean diameter) in the range of about 5 to about 30 μm, and tap density less than about 0.4 g/cm³, such that they possess an aerodynamic diameter of about 1 and about 3 micrometers, have been shown to display ideal properties for delivery to the deep lung. Larger aerodynamic diameters, ranging, for example, from about 3 to about 5 micrometers are preferred, however, for delivery to the central and upper airways. According to one embodiment of the invention the microparticles have a tap density of less than about 0.4 g/cm³ and a mean diameter of between about 5 μm and about 30 micrometers. According to another embodiment of the invention, the microparticles have a mass density of less than about 0.4 g/cm³ and a mean diameter of between about 5 micrometers and about 30 micrometers. In one embodiment of the invention, the microparticles have an aerodynamic diameter between about 1 micrometer and about 5 micrometers. In another embodiment of the invention, the microparticles have an aerodynamic diameter between about 1 micrometer and about 3 micrometers microns. In still another embodiment of the invention, the microparticles have an aerodynamic diameter between about 3 micrometers and about 5 micrometers.

For therapeutic, diagnosis or prophylactic use, microparticles can be delivered from an inhaler device, such as but not limited to a metered-dose-inhaler (MDI), dry-powder inhaler (DPI), nebulizer or by instillation. Such devices are known in the art. For example, a DPI is described in U.S. Pat. No. 4,069,819 issued to Valentini, et al. on Aug. 5, 1976.

Compositions Comprising Hydrophobic Derivatized Carbohydrates

In some embodiments of the present invention, the iloprost and/or another pharmaceutical agent to be administered in addition to iloprost is present in a composition comprising hydrophobic derivatized carbohydrates such as those described in U.S. Pat. No. 6,586,006, the disclosure of which is incorporated herein by reference in its entirety. For example, in some embodiments, the iloprost and/or another pharmaceutical agent to be administered in addition to iloprost is present in solid, glassy, delivery vehicles to obtain solid delivery systems. For such glassy formulations, the preferred density parameters discussed above with respect to some of the other types of microparticles described herein are not applicable. Likewise, for such glassy formulations, the preferred porosity parameters discussed above with respect to some of the other types of microparticles described herein are not applicable. In addition, for such glassy formulations, the preferred aerodynamic diameters discussed above with respect to some of the other types of microparticles described herein are not applicable. The choice of glassy delivery vehicles is determined by the nature of the iloprost and/or another pharmaceutical agent to be administered in addition to iloprost and the desired delivery rate of these compounds. A wide variety of delivery rates and types are provided herein. Preferred buffers, adjuvants and additional stabilizers are also provided. The delivery systems can be sized and shaped for a variety of modes of administration.

In some embodiments, the invention comprises rapidly soluble solid dose delivery systems comprising a stabilizing polyol (SP) and the iloprost and/or another pharmaceutical agent to be administered in addition to iloprost. These delivery systems can be formulated into powders of homogeneous particle size and larger, implantable forms.

In some embodiments, the iloprost and/or another pharmaceutical agent to be administered in addition to iloprost are provided in glassy vehicles formed from hydrophobically-derivatized carbohydrates (HDCs). These HDCs are non-toxic and the release of the iloprost and/or another pharmaceutical agent to be administered in addition to iloprost from these systems is highly controllable for the release of these compounds over extended time periods. The release from HDC delivery systems can be effected by devitrification, dissolution and/or hydrolysis.

The invention further encompasses coformulations of the different glassy vehicles to provide novel combination delivery systems. The combination delivery systems comprise HDCs combined with SPs and/or other slowly water soluble glassy materials, such as carboxylate, nitrate and phosphate glasses, to produce solid dose delivery systems with a wide variety of novel properties.

The invention encompasses solid dose delivery systems for multiphasic delivery comprising an outer portion comprising an HDC, slowly soluble in aqueous solution having a hollow compartment therein, and an inner portion residing in the compartment, the inner portion comprising at least one SP and a therapeutically effective amount of iloprost and/or another pharmaceutical agent to be administered in addition to iloprost.

The invention also encompasses methods of delivering iloprost and/or another pharmaceutical agent to be administered in addition to iloprost by providing the solid dose delivery systems described above and administering the system to a biological tissue. Administration can be by inhalation.

The invention further encompasses methods of making the solid dose delivery systems. The SP and/or HDC, iloprost and/or another pharmaceutical agent to be administered in addition to iloprost and any other components are mixed and processed by a wide variety of methods, including dissolving in the melt and subsequent quenching, spray drying, freeze drying, air drying, vacuum drying, fluidized-bed drying, co-precipitation and super-critical fluid evaporation. The resulting glass can be heated to soften and can then be extruded, drawn or spun into solid or hollow fibers. The dried components can also be mixed in aqueous or organic solutions and dried, such as by spray drying, freeze drying, air drying, vacuum drying, fluidized-bed drying, co-precipitation and super-critical fluid evaporation.

The invention further provides methods of making delivery systems suitable for slow or pulsatile release of iloprost and/or another pharmaceutical agent to be administered in addition to iloprost. The methods include combining iloprost and/or another pharmaceutical agent to be administered in addition to iloprost in solid solutions of stabilizing glass-forming polyols and/or HDCs and/or other glass formers with dissolution or degradation rates slower than that of the SP, and processing the components as described above. The ratio of materials can be controlled so as to provide a wide range of precisely defined release rates. The coformulations of SP and/or HDCs and other water-soluble and/or biodegradable glasses, plastics and glass modifiers produced thereby are also encompassed by the present invention.

The solid dose systems and methods of the invention also encompass solid dose forms which comprise fibers, spheres, tablets, discs, particles and needles of relatively homogeneous size distribution. The vehicles can be either microscopic or macroscopic.

Thus, some embodiments of the present invention comprise solid dose delivery systems comprising solid dose delivery vehicles and iloprost and/or another pharmaceutical agent to be administered in addition to iloprost. The delivery systems are formulated to provide precise delivery rates of the compounds incorporated therein. The delivery systems are particularly suitable for delivery of bioactive molecules to animals including humans.

Also encompassed by the invention are methods of delivery of iloprost and/or another pharmaceutical agent to be administered in addition to iloprost including administration by inhalation.

The invention also encompasses methods of making the delivery systems.

“Solid dose” as used herein, means that the iloprost and/or another pharmaceutical agent to be administered in addition to iloprost incorporated in the vehicle is in solid rather than liquid form and the solid form is the form used for delivery. By “effective amount” of iloprost and/or another pharmaceutical agent to be administered in addition to iloprost, is meant an amount to achieve the effect desired. For instance, with a bioactive material, an effective amount is one which effects the desired physiological reaction. The vehicle is in solid form and is amorphous or glassy in nature. Other additives, buffers, dyes etc. may be incorporated into the delivery systems. As used herein, the term “vehicle” includes all the glass-forming substances embodied in the described invention. The term “delivery system(s)” includes the solid dose forms comprising the vehicles and guest substances. Delivery systems formed from specific vehicles are given distinct names as indicated, unless otherwise indicated, the term delivery system encompasses each of these.

In one embodiment, the invention relates to solid dose systems with rapid release rates of the iloprost and/or another pharmaceutical agent to be administered in addition to iloprost. In this embodiment, the vehicle is a SP. SPs can be processed to obtain powders with homogeneous distribution of particle sizes in the form of either microspheres or needles. The SPs can also be processed to form macroscopic delivery forms suitable for formulation of implantable devices. A wide variety of dose forms and methods of making the dose forms are described herein. These SPs have been found to be particularly useful where otherwise denaturing conditions would render impossible the formulation of solid dosage forms of bioactive materials. In particular, such conditions include elevated temperatures (those above which the bioactive material is otherwise denatured) and the presence of organic solvents.

In another embodiment, the invention relates to solid dose systems with novel defined and controllable release rates of the iloprost and/or another pharmaceutical agent to be administered in addition to iloprost. In this embodiment, the vehicle is an organic carboxylate glass. Organic carboxylates form stable amorphous vehicles by solvent evaporation. These organic glasses release the incorporated iloprost and/or another pharmaceutical agent to be administered in addition to iloprost at precisely defined rates depending on the composite carboxylate anion and metal cation used. Like the vehicles comprising SPs, these glasses can be processed, either singly or in mixtures with other organic carboxylates and/or SPs and/or HDCs, to obtain powders with homogeneous particle size distribution, in the form of microspheres, microparticles or needles.

In a further embodiment, the invention relates to solid dose systems with novel defined and controllable release rates of the iloprost and/or another pharmaceutical agent to be administered in addition to iloprost. In this embodiment, the vehicle is a hydrophobic carbohydrate derivative (HDC). The rate of release of the iloprost and/or another pharmaceutical agent to be administered in addition to iloprost from the HDCs may be adjusted by selecting the carbohydrate, the hydrophobic moiety(ies) used to derivatize the carbohydrate and the degree of derivatization to provide the desired release rate. Like the vehicles comprising SPs, those comprising HDCs can be processed to obtain powders with homogeneous distribution of particle sizes in the form of microspheres, microparticles and needles. The HDCs can also be processed to form a wide variety of macroscopic delivery forms.

The dose forms and methods of making the dose forms are described herein. These delivery systems may be particularly useful where the nature of the iloprost and/or another pharmaceutical agent to be administered in addition to iloprost would render impossible the formulation of solid dosage forms as they provide delivery systems for compounds which are either difficult to formulate into dosage forms or to obtain effective physiologic concentrations of due to insolubility in aqueous solvents.

The delivery systems exist as solid solutions, emulsions, suspensions or coacervates of the iloprost and/or another pharmaceutical agent to be administered in addition to iloprost in the solid vehicle. The iloprost and/or another pharmaceutical agent to be administered in addition to iloprost may be resistant to higher temperatures within the vehicle than alone. The exact temperature resistance may depend on the vehicle used. Thus, the components of the delivery systems can be maintained as melts for brief periods without damaging the guest substances during processing. In the same way, the delivery systems can be further processed and may be resistant to damage during sintering with nitrate and/or carboxylate and/or HDCs and/or other glass-forming substances.

The invention further encompasses coformulations of various delivery vehicles and systems to provide a wide variety of combination delivery vehicles.

The present invention encompasses compositions and methods of making the compositions. Although singular forms may be used, more than one vehicle, more than one pharmaceutical agent and more than one additive may be present. Determination of the effective amounts of these compounds is within the skill of one in the art.

Stabilizing Polyol Delivery Systems

The invention encompasses solid dose delivery systems in which the delivery vehicle comprises a stabilizing polyol. These are termed “SP delivery systems”. SP delivery systems may be processed to a wide variety of solid dose forms particularly suited to therapeutic iloprost and/or another pharmaceutical agent to be administered in addition to iloprost.

SPs include, but are not limited to, carbohydrates. As used herein, the term “carbohydrates” includes, but is not limited to, monosaccharides, disaccharides, trisaccharides, oligosaccharides and their corresponding sugar alcohols, polysaccharides and chemically modified carbohydrates such as hydroxyethyl starch and sugar copolymers (Ficoll). Both natural and synthetic carbohydrates are suitable for use herein. Synthetic carbohydrates include, but are not limited to, those which have the glycosidic bond replaced by a thiol or carbon bond. Both D and L forms of the carbohydrates may be used. The carbohydrate may be non-reducing or reducing. Suitable vehicles are those in which a guest substance can be dried and stored without losses in significant activity by denaturation, aggregation or other mechanisms. Prevention of losses of activity can be enhanced by the addition of various additives such as inhibitors of the Maillard reaction as described below. Addition of such inhibitors is particularly preferred in conjunction with reducing carbohydrates.

Reducing carbohydrates suitable for use in the present invention are those known in the art and include, but are not limited to, glucose, maltose, lactose, fructose, galactose, mannose, maltulose, iso-maltulose and lactulose.

Non-reducing carbohydrates include, but are not limited to, trehalose, raffinose, stachyose, sucrose and dextran. Other useful carbohydrates include non-reducing glycosides of polyhydroxy compounds selected from sugar alcohols and other straight chain polyalcohols. The sugar alcohol glycosides are preferably monoglycosides, in particular the compounds obtained by reduction of disaccharides such as lactose, maltose, lactulose and maltulose. The glycosidic group is preferably a glucoside or a galactoside and the sugar alcohol is preferably sorbitol (glucitol). Particularly preferred carbohydrates are maltitol (4-O-β-D-glucopyranosyl-D-glucitol), lactitol (4-O-β-D-galactopyranosyl-D-glucitol), palatinit (a mixture of GPS, α-D-glucopyranosyl-1→6-sorbitol and GPM, α-D-glucopyranosyl-1→6-mannitol), and its individual sugar alcohols, components GPS and GPM.

Preferably, the SP is a carbohydrate that exists as a hydrate, including trehalose, lactitol and palatinit. Most preferably, the SP is trehalose. It has now been found that, surprisingly, solid dose delivery systems containing certain sugar hydrates like trehalose lack the “stickiness” or “tackiness” of solid dose forms containing other carbohydrates. Thus, for manufacture, packaging and administration, trehalose is the preferred SP.

Trehalose, (α-D-glucopyranosyl-α-D-glucopyranoside), is a naturally occurring, non-reducing disaccharide which was initially found to be associated with the prevention of desiccation damage in certain plants and animals which can dry out without damage and can revive when rehydrated. Trehalose has been shown to be useful in preventing denaturation of proteins, viruses and foodstuffs during desiccation. See U.S. Pat. Nos. 4,891,319; 5,149,653; 5,026,566; Blakeley et al. (1990) Lancet 336:854-855; Roser (July 1991) Trends in Food Sci. and Tech. 166-169; Colaco et al. (1992) Biotechnol. Internat., 345-350; Roser (1991) BioPharm. 4:47-53; Colaco et al. (1992) Bio/Tech. 10:1007-1011; and Roser et al. (May 1993) New Scientist, pp. 25-28, the disclosures of which are incorporated herein by reference in their entireties.

Other SPs suitable for use herein are described for instance in, WO 91/18091, 87/00196 and U.S. Pat. Nos. 4,891,319 and 5,098,893, the disclosures of which are incorporated herein by reference in their entireties, which describe the use of polyols as glasses for stabilizing molecules during drying and storage for reconstitution before use. Additionally, these polyols can be used in combination with other amorphous matrices to yield delivery systems which have desired release rates and characteristics which are readily and accurately controllable.

In some embodiments, iloprost and/or another pharmaceutical agent to be administered in addition to iloprost can be dried in trehalose from an organic/aqueous solvent mixture to give a coformulation that is now readily reconstituted in aqueous solvents. The present invention encompasses systems obtained in this manner. Methods of making the compositions obtained thereby are provided by the invention. The iloprost and/or another pharmaceutical agent to be administered in addition to iloprost is dissolved in an organic/aqueous solvent in combination with an effective amount of trehalose and then dried. This gives a solid solution, emulsion, suspension or coacervate of the iloprost and/or another pharmaceutical agent to be administered in addition to iloprost in a trehalose glass which then readily dissolves in an aqueous solution to give a finely dispersed suspension of the iloprost and/or another pharmaceutical agent to be administered in addition to iloprost. It has been shown that the immunosuppressant CSA (which is poorly soluble in water and normally administered as an oil emulsion) in a solution of trehalose in a 1:1 ethanol:water mixture can be dried to give a clear glass of trehalose containing CSA. This glass can be milled to give a free flowing powder, which can also be tabletted, which when added to water dissolves instantaneously to give a finely dispersed suspension of CSA in water.

HDC Delivery Systems

The invention further encompasses delivery systems in which the vehicle contains at least one HDC. These are termed “HDC delivery systems”. HDCs form a separate group of non-toxic carbohydrate derivatives suitable for use in forming the vehicle. The invention thus encompasses the glassy form of these HDCs which is also referred to as an amorphous matrix-forming composition. The HDC delivery systems are particularly suited for use in controlled, pulsatile or delayed release of iloprost and/or another pharmaceutical agent to be administered in addition to iloprost. Any of the pharmaceutical agents described herein may be incorporated in the HDC delivery systems.

As shown herein, HDCs readily form glasses either from a quenched melt or from an evaporated organic solvent. The HDCs can also be processed by the methods described for the SPs.

As used herein, HDC refers to a wide variety of hydrophobically derivatized carbohydrates where at least one hydroxyl group is substituted with a hydrophobic moiety including, but not limited to, esters and ethers. Numerous examples of suitable HDCs and their syntheses are described in Developments in Food Carbohydrate—2 ed. C. K. Lee, Applied Science Publishers, London (1980), the disclosure of which is incorporated herein by reference in its entirety. Other syntheses are described for instance, in Akoh et al. (1987) J. Food Sci. 52:1570; Khan et al. (1993) Tetra. Letts 34:7767; Khan (1984) Pure & Alpl. Chem. 56:833-844; and Khan et al. (1990) Carb. Res. 198:275-283, the disclosures of which are incorporated herein by reference in their entireties. Specific examples of HDCs include, but are not limited to, sorbitol hexaacetate (SHAC), α-glucose pentaacetate (α-GPAC), β-glucose pentaacetate (β-GPAC), 1-0-Octyl-β-D-glucose tetraacetate (OGTA), trehalose octaacetate (TOAC), trehalose octapropanoate (TOPR), sucrose octaacetate (SOAC), cellobiose octaacetate (COAC), raffinose undecaacetate (RUDA), sucrose octapropanoate, cellobiose octapropanoate, raffinose undecapropanoate, tetra-O-methyl trehalose and di-O-methyl-hexa-O-acetyl sucrose. An example of a suitable HDC where the carbohydrate is trehalose is:

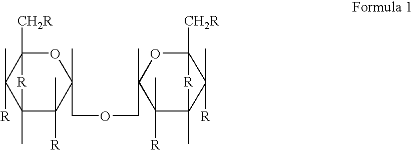

In formula 1, R represents a hydroxyl group, or less hydrophilic derivative thereof, such as an ester or ether or any functional modifications thereof where at least one R is not hydroxyl but a hydrophobic derivative. Suitable functional modifications include, but are not limited to, where the oxygen atom is replaced by a heteroatom, such as N or S. The degree of substitution can also vary, and may be a mixture of distinct derivatives. Full substitution of the hydroxyl groups need not occur and provides an option to alter physical properties (such as solubility) of the vehicle. R can be of any chain length from C₂ upwards and may be straight, branched, cyclic or modified. While formula 1 depicts the disaccharide trehalose, any of the carbohydrates discussed herein may be the carbohydrate backbone and the position of the glycosidic linkage and saccharide chain length can vary. Typically, the practical range in terms of cost and efficiency of synthesis is a pentasaccharide; however, the invention is not limited to saccharides of any particular type, glycosidic linkage or chain length. Various other aspects of the HDCs are not limiting. For instance, the component saccharides of each HDC can also be varied, the position and nature of the glycosidic bonding between the saccharides may be altered and the type of substitution can vary within an HDC. A representative example of a HDC with mixed substitution with esters and ethers is 1-o-Octyl-β-D-glucopyranoside 2,3,4,5-tetraacetate: 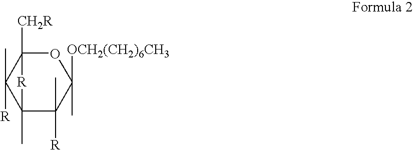
where R is O₂CCH₃.

The ability to modify the properties of HDCs by slight alterations in composition renders them uniquely suited to administer iloprost and/or another pharmaceutical agent to be administered in addition to iloprost. The HDC delivery systems can be tailored to have precise properties such as release rates of iloprost and/or another pharmaceutical agent to be administered in addition to iloprost. Such tailoring can be by varying the modifications of a particular carbohydrate or by combining a variety of different HDCs.

Pure single HDC glasses are stable at ambient temperatures and up to at least 60% humidity. Thus, pure single HDC glasses or mixtures of HDC glasses may provide beneficial levels of stability of the iloprost and/or another pharmaceutical agent to be administered in addition to iloprost.

The HDC glasses can be formed either from evaporation of the solvent or by quenching of the HDC melt. Because of the low softening points of certain HDC glasses, thermally labile pharmaceutical agents such as drugs and biological molecules can be incorporated into the HDC melt during processing of the delivery system without decomposition. Surprisingly, these pharmaceutical agents have demonstrated zero order release kinetics when the amorphous matrix forming compositions erode in aqueous solution. Release follows the process of surface devitrification. The HDC delivery systems can be easily modeled into any shape or form, such as those described herein. Such modeling can be by extrusion, molding etc. by any method known in the art. The HDC delivery vehicles are non-toxic and inert to any solutes which may be incorporated therein.

These HDC delivery systems, when formulated as matrices and/or coatings, undergo heterogeneous surface erosion when placed in an aqueous environment. While not being bound by any one theory, one possible mechanism for their degradation begins with an initial surface devitrification as supersaturation occurs at the interface, followed by subsequent erosion and/or dissolution of the surface layers at a slower rate. The matrices can be modified by careful selection of components to give the desired devitrification rates and hence the required release rates of the iloprost and/or another pharmaceutical agent to be administered in addition to iloprost as the devitrified matrix provides no barrier to the release of the iloprost and/or another pharmaceutical agent to be administered in addition to iloprost.

The HDC melts are excellent solvents for many organic molecules. This makes them particularly suitable for use in delivery of bioactive materials otherwise difficult to formulate. More than 20% weight percent of organic molecules can be incorporated into the HDC delivery systems. Notably, HDCs are inert and show no reactivity to their solutes or guest substances incorporated therein. As described in more detail below, the HDCs are suitable for forming a dispersion of a fine suspension of a SP delivery system to yield complex, composite delivery systems.

Component HDCs are synthesized to high purity using established chemical or enzymic synthetic principles. The HDCs and iloprost and/or another pharmaceutical agent to be administered in addition to iloprost may be intimately mixed together in the appropriate molar ratios and melted until clear. Suitable melting conditions include, but are not limited to, melting in open glass flasks between 100 and 150° C. for 1-2 minutes. This results in a fluid melt which may be allowed to slightly cool before, dissolving the guest in the melt if required, quenching to glass for instance by pouring over a brass plate or into a metal mould for shaped delivery vehicles. Either way, melt temperature can be carefully controlled and guest substances can be incorporated into either the pre-melted HDC formulation, or stirred into the cooling HDC melt before quenching.

The HDC melts are thermally stable and allow the incorporation of organic molecules without denaturation or suspension of core particles without alteration of their physical nature. The glass melts can also be used to coat micron-sized particles, this is particularly important in the formulation of non-hygroscopic powders containing hygroscopic actives, for by-inhalation administration of therapeutic agents.

Alternatively, vitreous HDC delivery vehicles can be formed by evaporation of the HDC and iloprost and/or another pharmaceutical agent to be administered in addition to iloprost to be incorporated in solution in a solvent or mixture of solvents. Component HDCs are readily dissolved in many organic solvents. Suitable solvents include, but are not limited to, dichloromethane, chloroform, dimethylsulfoxide (DMSO), dimethylformamide (DMF) and higher alcohols. The nature of the solvent is immaterial as it is completely removed on formation of the delivery system. Preferably both the component HDC and guest substance are soluble in the solvent. However, the solvent may dissolve the HDC and allow a suspension of the guest substance. On concentrating the solvent, crystallization does not occur with the more useful HDCs. Instead, an amorphous solid is produced, which has similar properties to the quenched glass. Again, guest substances can be easily incorporated either from solution or as a particle suspension.

HDC glass transition temperatures (Tg) are low, typically less than 70° C. and, surprisingly, are not predictable from the melt temperatures. In general, the tendency to crystallize, from a cooling melt or with reducing solvent, is low. Both devitrification and the fluidity of the melt at temperatures close to Tg, can be controlled by modifiers such as other derivative sugars and certain organic actives. The following two tables provide Tg and melting temperature data for a variety of HDCs suitable for use, either alone, or in a composite glass, herein.

TABLE 1

| Material/Glass | M.Pt./° C. | Tg/° C. | M.Wt |
|---|---|---|---|
| SHAC | 100-104 | −6 | 434.4 |
| α-GPAC | 109-111 | 14 | 390.3 |
| β-GPAC | 130-131 | 17 | 390.3 |
| OGTA | 50-52 | −10 | 460.5 |
| TOAC | 101-103 | 50 | 678.6 |
| TOPR | 47-48 | 3 | 790.6 |
| SOAC | 87-89 | 25 | 678.6 |
| COAC | 224-226 | 65 | 678.6 |
| RUDA | 87-88 | 55 | 966.9 |

TABLE 2

| | Mole ratios | |
|---|---|---|
| Glass System | HDCs in glass | Tg/° C. |
| TOAC | 100 | 50 |
| RUDA | 100 | 55 |
| α-GPAC:TOAC | 10:90 | 47 |
| | 25:75 | 44 |
| | 50:50 | 32 |
| | 75:25 | 22 |
| SOAC:TOAC | 25:75 | 41 |
| COAC:TOAC | 25:75 | 55 |
| TOPR:TOAC | 22:78 | 37 |
| RUDA:TOAC | 10:90 | 52 |
| | 25:75 | 53 |
| | 50:50 | 52 |
| | 75:25 | 54 |

The invention further encompasses delivery vehicles comprising combinations of different HDCs which have now been found to provide novel delivery vehicles with highly controllable Tg and other physicochemical properties such as viscosity and resistance to aqueous degradation.

Combination Delivery Systems

The invention also encompasses solid dose delivery systems comprising HDCs and SPs and/or other glass forming substances in coformulations and other combinations. These are termed “combination delivery systems”.

At least two combination delivery systems are produced by the coformulation of HDC and SP vehicles to produce the delivery systems. In one instance, microspheres of the SP delivery system are suspended within the HDC delivery system. In the second instance, microspheres of the HDC delivery system are suspended in the SP delivery system. These combination delivery systems allow release of at least two different pharmaceutical agents, one hydrophobic and one hydrophilic, at least two different release rates.

Other combination delivery systems are formed by coating one delivery system with another. For instance, an SP delivery system in implantable form could be coated with a layer of HDC or HDC delivery system to provide delayed release of the iloprost and/or another pharmaceutical agent to be administered in addition to iloprost in the SP delivery system or sequential release of different pharmaceutical agents. A variety of such forms can be readily envisioned. The number of coatings is theoretically unlimited and is within the skill of one in the art to determine.

Other Components in the Delivery Systems Other Glasses

As discussed below, the delivery systems may further contain at least one physiologically acceptable glass. Suitable glasses include, but are not limited to, carboxylate, phosphate, nitrate, sulfate, bisulfate, HDCs and combinations thereof. Carboxylates have previously been used where slowly water soluble glasses are required as many of these are only poorly soluble in water. Suitable such glasses include, but are not limited to, those described in PCT/GB 90/00497, the disclosure of which is incorporated herein by reference in its entirety. However, the formation of these carboxylate glasses has previously only been done by quenching of the melt. The elevated temperature necessary to melt the carboxylates severely limits the carboxylates that can be used to form vitreous delivery vehicles, particularly in the case of bioactive materials which tend to be heat labile. Carboxylate glasses can be easily formed by evaporation of a solvent containing the glass-forming metal carboxylate and the pharmaceutical agents to be incorporated. The invention thus encompasses methods of making vehicles and systems comprising dissolving a carboxylate component in a suitable solvent therefor and evaporating the solvent to yield a vitreous glass. Mixtures of carboxylates can be used as can mixtures of other glass-forming components to produce novel delivery systems which are encompassed by the present invention.

The delivery systems may also be coated with one or more layers of a physiologically acceptable glass having a predetermined solution rate. This is especially effective for pulsatile release of pharmaceutical agents. The composition may further contain other water soluble and biodegradable glass formers. Suitable glass formers include, but are not limited to, lactide and lactide/glycolide copolymers, glucuronide polymers and other polyesters, polyorthoesters, and polyanhydrides.

The compositions of the present invention may include iloprost and/or any of the pharmaceutical agents to be administered in addition to iloprost described herein. Preferably, if the pharmaceutical agents and/or vehicle contain carboxyl and amino, imino or guanidino groups, the delivery systems further comprise at least one physiologically acceptable inhibitor of the Maillard reaction in an amount effective to substantially prevent condensation of amino groups and reactive carbonyl groups in the composition.

The inhibitor of the Maillard reaction can be any known in the art. The inhibitor is present in an amount sufficient to prevent, or substantially prevent, condensation of amino groups and reactive carbonyl groups. Typically, the amino groups are present on the bioactive material and the carbonyl groups are present on the carbohydrate, or the converse. However, the amino and carbonyl groups may be intramolecular, within either the biological substance or the carbohydrate. Various classes of compounds are known to exhibit an inhibiting effect on the Maillard reaction and hence to be of use in the compositions described herein. These compounds are generally either competitive or noncompetitive inhibitors. Competitive inhibitors include, but are not limited to, amino acid residues (both D and L), combinations of amino acid residues and peptides. Particularly preferred are lysine, arginine, histidine and tryptophan. Lysine and arginine are the most effective. There are many known noncompetitive inhibitors. These include, but are not limited to, aminoguanidine and derivatives, are 4-hydroxy-5,8-dioxoquinoline derivatives and suitable Maillard inhibitors such as those in EP-A-O 433 679, the disclosure of which is incorporated herein by reference in its entirety.

Dosage Forms

In addition to the dosage forms described above, a variety of other dosage forms suitable for different uses are provided herein.

Some embodiments of the delivery systems include microspheres. In some embodiments, the microspheres have a narrow size distribution. The microspheres may have any dimensions described in the present application. In some embodiments, the microspheres have a mass median aerodynamic diameter (MMAD) of 0.1 to 10 microns. More preferably, the mass median aerodynamic diameter is 0.5 to 5 microns. Most preferably, mass median aerodynamic diameter is 1 to 4 microns. In particular for pulmonary administration, the preferred mass median aerodynamic diameter is 1.5-3 microns.

An alternative embodiment of the delivery vehicle in the invention comprises a hollow vehicle comprised of poorly water soluble glass or plastic which is filled and optionally coated the delivery systems described herein.

In another embodiment of the invention, coformulations of vehicles and other poorly water soluble materials are included. For example, coformulations of vehicles with water-soluble glasses such as phosphate, nitrate or carboxylate glasses or biodegradable plastics such as lactide or lactide/glycolide copolymers will yield a more slowly eroding vehicle for delayed release of the bioactive material.

Methods of Making the Delivery Systems

The invention further encompasses methods of making the delivery systems. Providing the exposure time is limited, iloprost and/or another pharmaceutical agent to be administered in addition to iloprost admixed in dry vehicles can be heated to fluidize the glass which can then be drawn or spun as a fiber without damage to the product. Fibers can either be drawn from a billet, cooled to solidify them and then wound onto a drum or they can be spun through fine holes in a rapidly rotating cylinder that is heated above the melting point of the vehicle. Being inherently brittle, these fibers can be readily cut, broken, crushed or chopped into short lengths to form long cylindrical rods or needles. By varying the diameter of the fibers produced, needles can be formed which vary from micro to macro needles, i.e., from thicknesses of a few microns to fractions of a millimeter. It has been found that cotton candy machines are suitable for use in preparing the finer diameter microfibers. Although the optimal conditions must be determined empirically for each vehicle, such determinations are well within the skill of one in the art.

To prepare microspheres of the present invention, several methods can be employed depending upon the desired application of the delivery vehicles. Suitable methods include, but are not limited to, spray drying, freeze drying, air drying, vacuum drying, fluidized-bed drying, milling, co-precipitation and super-critical fluid evaporation. In the case of spray drying, freeze drying, air drying, vacuum drying, fluidized-bed drying and super-critical fluid evaporation, the components (SP and/or HDC, and/or other glass former, guest substances, buffers etc.) are first dissolved or suspended in suitable solvents. In the case of milling, glasses formed from the components, either by solvent evaporation or quenching of the melt, are milled in the dried form and processed by any method known in the art. In the case of co-precipitation, the components are mixed in organic conditions and processed as described below.

Spray drying can be used to load the vehicle with the guest substance. The components are mixed under suitable solvent conditions and dried using precision nozzles to produce extremely uniform droplets in a drying chamber. Suitable spray drying machines include, but are not limited to, Buchi, NIRO, APV and Lab-plant spray driers used according to the manufacturer's instructions. A number of carbohydrates are unsuitable for use in spray drying as the melting points of the carbohydrates are too low, causing the dried amorphous materials to adhere to the sides of the drying chamber. Generally, carbohydrates with a melting point of less than the operating temperature of the spray drying chamber are unsuitable for use in spray drying. For example, palatinit and lactitol are not suitable for use in spray drying under conventional conditions. A determination of suitable carbohydrates can thus be made on known melting points or determined empirically. Such determinations are within the skill of one in the art.

An alternative method for manufacturing microspheres as delivery vehicles in accord with the present invention is to prepare a uniform aqueous/organic phase emulsion of the guest substance in a solution of the vehicle as the aqueous phase and a glass former in the organic phase or the converse. This is followed by drying of the emulsion droplets to form a solid solution of the guest substance and vehicle in an amorphous matrix of the glass former. In a modification of this method, the emulsion may be formed from the iloprost and/or another pharmaceutical agent to be administered in addition to iloprost in solid solution in the vehicle and two different glass formers and/or polymers dissolved together in one solvent, or dissolved into two separate solvents. The solvent(s) are then removed by evaporation to yield double or multi-walled microspheres. Suitable methods for making multi-walled microspheres are described, for instance, in Pekarek et al. (1994) Nature 367:258-260; and U.S. Pat. No. 4,861,627.

The delivery system can also be dried from an organic solution of an SP and a hydrophobic guest substance to form a glass containing homogeneously distributed guest substance in solid solution or fine suspension in the polyol glass. These glasses can then be milled and/or micronized to give microparticles of homogeneous defined sized.

The iloprost and/or another pharmaceutical agent to be administered in addition to iloprost and vehicle can also be co-precipitated to give high quality powders. Co-precipitation is performed by spraying, for instance with an air brush, the various components and/or polymeric glass former into a liquid in which neither dissolves, such as ice-cold acetone.

An alternative embodiment of the delivery vehicle in the invention comprises a hollow vehicle comprised of poorly water soluble glass or plastic which is filled and optionally coated with SP and/or HDC glass and the iloprost and/or another pharmaceutical agent to be administered in addition to iloprost. Fine hollow fibers of slowly water-soluble inorganic or organic glasses can be drawn from a hollow billet and a finely powdered SP delivery system can be incorporated into the lumen of the billet, and therefore of the fiber, during the process.

In another embodiment of the invention, coformulations of vehicles and other water soluble materials are included. For example, coformulations of vehicles with water-soluble glasses such as phosphate glasses (Pilkington Glass Company) or biodegradable plastics such as lactide or lactide/glycolide copolymers will yield a more slowly eroding vehicle for delayed release of the guest substance. To produce the coformulations, a finely powdered glass containing the guest substance can be intimately mixed with a finely powdered carboxylate glass and co-sintered. Alternatively, if a metal carboxylate glass has a lower melting point than the delivery system, the latter can be homogeneously embedded as an encapsulate in a carboxylate glass on quenching of the melt obtained. This can be milled to give a fine powder with solubilities intermediate between the relatively rapid solubility of the vehicle and the slow solubility of the carboxylate glass.

Alternate coformulations include the use of a homogeneous suspension of the finely powdered vitreous delivery system encapsulated in a carboxylate glass by drying from an organic solvent in which the carboxylate is soluble, but the amorphous powder is not, to form the carboxylate glass. This can be ground to give a fine powder which would have the relatively rapidly dissolving delivery system entrapped within a slow dissolving carboxylate glass (i.e., comparable to a conventional slow-release system). Pulsatile release formats can be achieved either by repeated encapsulation cycles using glasses of different dissolution rates, or by mixing powders of a number of coformulations with the desired range of release characteristics. Note that this glass could also be drawn or spun to give microfibers or microneedles which would be slow-release implants. It will be appreciated that any delivery system formulation should be such that it is capable of releasing the iloprost and/or another pharmaceutical agent to be administered in addition to iloprost upon administration, and should not unduly effect the stability of the material being administered.

As discussed above, glasses of derivatized carbohydrates are also suitable for use herein. Suitable derivatized carbohydrates include, but are not limited to, carbohydrate esters, ethers, imides and other poorly water-soluble derivatives and polymers.

The delivery vehicle can be loaded with the iloprost and/or another pharmaceutical agent to be administered in addition to iloprost by drying a solution of the iloprost and/or another pharmaceutical agent to be administered in addition to iloprost containing a sufficient quantity of vehicle to form a glass on drying. This drying can be accomplished by any method known in the art, including, but not limited to, freeze drying, vacuum, spray, belt, air or fluidized-bed drying. The dried material can be milled to a fine powder before further processing the material with the polyol glass or coformulation.

Different dosing schemes can also be achieved depending on the delivery vehicle employed. A delivery vehicle of the invention can provide for a quick release or flooding dose of the iloprost and/or another pharmaceutical agent to be administered in addition to iloprost after administration, where the delivery system is readily soluble. Coformulations of vehicles with slowly water soluble glasses and plastics such as phosphate, nitrate or carboxylate glasses and lactide/glycolide, glucuronide or polyhydroxybutyrate plastics and polyesters, can provide more slowly dissolving vehicles for a slower release and prolonged dosing effect. A priming and booster effect can also be realized by utilizing a hollow, slowly water soluble vehicle filled and coated with a rapidly dissolving SP and/or HDC glass loaded with the guest substance. The glass coating loaded with the iloprost and/or another pharmaceutical agent to be administered in addition to iloprost will dissolve rapidly to give an initial dosing effect. There will be no dosing action while the hollow outer wall portion of the vehicle dissolves, but the initial priming dose will be followed by a booster dose of the inner filling when the hollow outer wall is breached by dissolution. Such pulsatile release format is particularly useful for delivery of immunogenic compositions. Should multiple effect pulsatile delivery be desirable, delivery vehicles with any combination of layers of “non-loaded” vehicles and vehicles loaded with the iloprost and/or another pharmaceutical agent to be administered in addition to iloprost can be constructed.

The delivery of more than one pharmaceutical agent can also be achieved using a delivery system comprised of multiple coatings or layers of the vehicle loaded with different materials or mixtures thereof. Administration of the delivery systems of the present invention can be used in conjunction with other conventional therapies and coadministered with other therapeutic, prophylactic or diagnostic substances.

Methods of Delivery

The invention further encompasses methods of delivery of the delivery systems.

Compositions suitable for by-inhalation administration include, but are not limited to, powder forms of the delivery systems. Preferably the powders are of a particle size with mass median aerodynamic diameter (MMAD) of 0.1 to 10 microns. More preferably, the mass median aerodynamic diameter is 0.5 to 5 microns. Most preferably, particle size is 1 to 4 microns. In particular for pulmonary administration, the preferred mass median aerodynamic diameter is 1.5-3 microns.

Preferably SP delivery vehicle powders also contain an effective amount of a physiologically acceptable molecular water pump buffer (MWPB). A MWPB is a physiologically acceptable salt that effects a loss of water from the composition so that at ambient humidity the vapor pressure of water of crystallization is at least 14 mm Hg (2000 Pa) at 20° C. and does not interfere with glass formation of the vehicle. An effective amount of an MWPB is one which sufficiently reduces hygroscopicity to prevent substantial clumping, for instance, a 50% molar ratio of potassium sulfate. Sodium sulfate and calcium lactate are the preferred salts with potassium sulfate being the most preferred.

The composite HPC delivery systems are particularly useful for by-inhalation dosage forms. For instance, 10% (w/v) αGPAC/TOAC mixed delivery systems are resistant to 95% relative humidity (RH) but recrystallize on contact with liquid water and thus release any iloprost and/or another pharmaceutical agent to be administered in addition to iloprost incorporated therein. This is especially important for inhalable powders as these powders would preferably devitrify and release the pharmaceutical agents upon hitting liquid in the alveoli and not in the humid tracheal airways.

Atomizers and vaporizers filled with the powders are also encompassed by the invention. There are a variety of devices suitable for use in by-inhalation delivery of powders. See, e.g., Lindberg (1993) Summary of Lecture at Management Forum 6-7 Dec. 1993 “Creating the Future for Portable Inhalers”, the disclosure of which is incorporated herein by reference in its entirety. Additional devices suitable for use herein include, but are not limited to, those described in WO 94/13271, WO 94/08552, WO 93/09832 and U.S. Pat. No. 5,239,993, the disclosures of which are incorporated herein by reference in their entireties.

In some embodiments of the present invention, the iloprost and/or another pharmaceutical agent to be administered in addition to iloprost may be administered using the methods and compositions described in U.S. Pat. No. 6,517,860, the disclosure of which is incorporated herein by reference in its entirety.

In one embodiment of the present invention, the compositions contain iloprost and/or another pharmaceutical agent to be administered in addition to iloprost and hydrophobically-derivatized (substituted) carbohydrates (HDCs) in powder form. In another embodiment, the dosage forms contain iloprost and/or another pharmaceutical agent to be administered in addition to iloprost, HDCs and surfactants in powder form. The compositions form solid solutions, suspensions or emulsions of iloprost and/or another pharmaceutical agent to be administered in addition to iloprost, with or without modifiers and/or other additives, in an HDC glass.

The invention also encompasses methods of making compositions of suspensions of iloprost and/or another pharmaceutical agent to be administered in addition to iloprost in aqueous solvents and the compositions obtained thereby. The methods include obtaining the compositions described above and dispersing the glass in an aqueous solvent suitable for administration. The compositions obtained thereby are also suitable for use as a solid dose form.

The compositions described herein are also suitable for delivery of pharmaceutical agents including hydrophobic agents.

The compositions of the present invention are readily formulated into glasses suitable as dosage forms with increased bioavailability for mucosal delivery of iloprost and/or another pharmaceutical agent to be administered in addition to iloprost. The dosage forms described herein may be tailored for delivery to different mucosal surfaces allow for increased bioavailability of bioactive agents, particularly hydrophobic drugs. For instance, in some embodiments, a solution mimicking lung surfactant allows for release of pharmaceutical agents from the compositions described herein which lack a surface active agent. This is in contrast to the lack of release of these same formulations in saline. Alternatively, in some embodiments surfactants may be included in the compositions comprising iloprost and/or another pharmaceutical agent to be administered in addition to iloprost.

In some embodiments, the invention encompasses methods of making glasses for use in making dosage forms providing increased bioavailability of pharmaceutical agents through mucosal delivery. The glasses contain iloprost and/or another pharmaceutical agent to be administered in addition to iloprost and a surface active agent in solid solution, suspension or emulsion phase of HDCs. Hydrophilic surfactants, i.e. those with a high hydrophile-lipophile balance (HLB), readily form a continuous phase with these HDC glasses which are stable during processing and storage. In some embodiments, these glasses release more pharmaceutical agent in aqueous buffers than matrices not containing surfactants. These “solid solutions” are highly stable; they show no sign of phase separation for up to 4 weeks at room temperature and the hydrophobic pharmaceutical agent incorporated therein can be quantitatively extracted by organic solvent extraction and shows no evidence of degradation of analysis by HPLC. The glass obtained can be in the vitreous or crystalline form or mixtures thereof. The glass can also be an amorphous matrix. As used herein, “glass”, glasses” or “glassy” refers to all of these embodiments.

The invention thus encompasses compositions of pharmaceutical agents and HDCs in powder form. The invention further encompasses compositions of pharmaceutical agents, HDCs and surface active agents in powder form. These powders can be made either from the melt of the HDC incorporating the bioactive agent or by evaporation from non-aqueous solutions of the HDC and the bioactive agent. The compositions obtained from the melt can be processed to a powder by any method known in the art such as milling. The powders are suitable for use as solid dose forms or can be further processed into tablets or other dosage forms.

The invention further encompasses compositions of hydrophobic pharmaceutical agents, HDCs and surface active agents. The compositions form solid suspensions, solutions or emulsions. The compositions obtained thereby are suitable for use as dosage forms or can be processed into other forms such as powders.

The invention also encompasses methods of making compositions of stable formulations of hydrophobic, bioactive agents in aqueous solution and the compositions obtained thereby. The methods include obtaining the glasses described above and dispersing the solid phase in an aqueous solvent. The compositions obtained thereby are also suitable for use as a pharmaceutical dosage form.

Any of the pharmaceutical agents discussed herein may be administered in addition to iloprost using the methods and compositions described herein. As discussed above, the pharmaceutical agents to be administered in addition to iloprost may be present in the same composition as the iloprost or may be in separate compositions. In addition, as discussed above, the pharmaceutical agents to be administered at the same time as iloprost, or before or after the administration of iloprost.

HDCs form a group of non-toxic carbohydrate derivatives. HDCs readily form glasses either from a quenched melt or from an evaporated organic solvent. The HDCs can also be processed by the methods known in the art and described for other carbohydrate dosage forms.

As used herein, HDC refers to a wide variety of hydrophobically derivatized carbohydrates where at least one hydroxyl group is substituted with a hydrophobic moiety including, but not limited to, esters and ethers. Numerous examples of suitable HDCs and their syntheses are described in Developments in Food Carbohydrate—2 ed. C. K. Lee, Applied Science Publishers, London (1980); and PCT publication No. 96/03978. Other syntheses are described for instance, in Akoh et al. (1987) J. Food Sci. 52:1570; Khan et al. (1933) Tetra. Letts 34:7767; Khan (1984) Pure & Appl. Chem. 56:833-844; and Khan et al. (1990) Carb. Res. 198:275-283, the disclosures of which are incorporated herein by reference in their entireties. Specific examples of HDCs include, but are not limited to, sorbitol hexaacetate (SHAC), α-glucose pentaacetate (α-GPAC), β-glucose pentaacetate (β-GPAC), 1-O-Octyl-β-D-glucose tetraacetate (OGTA), trehalose octaacetate (TOAC), trehalose octapropionate (TOP), trehalose octa-3,3,dimethylbutyrate (TO33DMB), trehalose diisobutyrate hexaacetate, trehalose octaisobutyrate, lactose octaacetate, sucrose octaacetate (SOAC), cellobiose octaacetate (COAC), raffinose undecaacetate (RUDA), sucrose octapropanoate, cellobiose octapropanoate, raffinose undecapropanoate, tetra-O-methyl trehalose, trehalose octapivalate, trehalose hexaacetate dipivalate and di-O-methyl-hexa-O-actyl sucrose and mixtures thereof. An example of a suitable HDC where the carbohydrate is trehalose is:

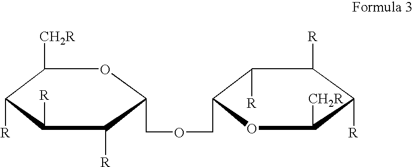

In the above formula, R represents a hydroxyl group, or less hydrophilic derivative thereof, such as an ester or ether or any functional modifications thereof where at least one R is not hydroxyl but a hydrophobic derivative. Suitable functional modifications include, but are not limited to, replacing the oxygen atom with a heteroatom, such as N or S. The degree of substitution can also vary, and can be a mixture of distinct derivatives and/or linkages. Full substitution of the hydroxyl groups need not occur and provides an option to alter physical properties (such as solubility) of the vehicle. R can be of any chain length from C₂ upwards and can be straight, branched, cyclic or modified and mixtures thereof. While formula 3 depicts the disaccharide trehalose, any of the carbohydrates discussed herein can be the carbohydrate backbone and the position of the glycosidic linkage and saccharide chain length can vary. Typically, the practical range in terms of cost and efficiency of synthesis is a pentasaccharide; however, the invention is not limited to saccharides of any particular type, glycosidic linkage or chain length. Various other aspects of the HDCs are not limiting. For instance, the component saccharides of each HDC can also be varied, the position and nature of the glycosidic bonding between the saccharides can be altered and the type of substitution can vary within an HDC.

The ability to modify the properties of HDCs by slight alterations in chemical structure renders them uniquely suited to use as delivery vehicles for bioactive agents, particularly compared to polymeric systems which often depend on regions of crystallinity to vary their properties, particularly bioerosion. The HDC vehicles can be tailored to have precise properties such as well-defined release rates of bioactive agents. Such tailoring can be by varying the modifications of a particular carbohydrate or by combining a variety of different HDCs.

Pure single HDC glasses have been found to be stable at ambient temperatures and up to at least 60% humidity and even mixtures of HDC glasses incorporating certain bioactive agents are stable at ambient temperatures and up to at least 95% humidity. Incorporation of even 10% (w/v) of extremely hygroscopic pharmaceutical agents, such as the synthetic corticosteroids, yields HDC glasses that are stable when exposed to relative humidities of up to 95% at room temperature for over a month, yet immediately release the bioactive agents within 5-10 minutes upon addition to aqueous solvents.

Adding other HDCs at these same levels to the formulations also produced mixed HDC glasses that were equally resistant to devitrification at 95% relative humidity. The ability to tailor the dissolution rates of composite HDC glasses makes them particularly useful as controlled release delivery vehicles for mucosal delivery.

The HDC glasses can be formed either from evaporation of the solvent or by quenching of the HDC melt. Because of the low softening points of certain HDC glasses, thermally labile pharmaceutical agents can be incorporated into the HDC melt during processing without decomposition. Surprisingly, these bioactive agents have demonstrated zero order release kinetics when the forming compositions erode in aqueous solution. When the composition is in vitreous form, release follows the process of surface devitrification. The HDC vehicles can be easily modeled into any shape or form, such as those described herein. Such modeling can be by extrusion, molding etc. by any method known in the art. The HDCs are suitable for use as delivery vehicles as they are non-toxic and inert to any solutes which can be incorporated therein.

The vitreous forms of the compositions undergo heterogeneous surface erosion when placed in an aqueous environment, making the compositions particularly suited to mucosal delivery. While not being bound by any one theory, one possible mechanism for degradation of the compositions begins with an initial surface devitrification as supersaturation occurs at the interface, followed by subsequent erosion and/or dissolution of the surface layers at a slower rate. The compositions can be modified by careful selection of components to give the desired devitrification rates and hence the required release rates of the pharmaceutical agent as the devitrified layer provides no barrier to the release of the pharmaceutical agent.

The incorporation and release of hydrophobic pharmaceutical agents from the compositions can be enhanced by the incorporation of surface active agents during the formation of the compositions. Suitable surfactants are those with a high HLB, i.e., those that are hydrophilic with a HLB of at least about 3. Preferably, the surfactants are dry at room temperature. Suitable surfactants include, but are not limited to, glyceryl monostearate, sorbitan monolaurate, polyoxyethylene-4-lauryl ether, polyethylene glycol 400 monostearate, polyoxyethylene-4-sorbitan monolaurate, polyoxyethylene-20-sorbitan monopalmitate, polyoxyethylene-40-stearate, sodium oleate and sodium lauryl sulfate. Suitable surfactants also include lung surfactants both naturally derived and synthetically manufactured. A suitable artificial lung surfactant is described for instance in Bangham et al. (1979) Biochim. Biophys. Acta 573:552-556, the disclosure of which is incorporated herein by reference in its entirety. Suitable concentrations of surfactants can be empirically derived as described in Example 17.

In one embodiment, the dosage forms are in the form of a powder. These are particularly suitable for use in by-inhalation delivery systems. Preferably the powders are of a mass median aerodynamic diameter of about 0.1 to about 10 microns. More preferably, the mass median aerodynamic diameter is about 0.5 to about 5 microns. Most preferably, mass median aerodynamic diameter is about 1 to 4 about microns. In particular for pulmonary administration, the preferred mass median aerodynamic diameter is about 1.5-about 3 microns.

The compositions can be formulated into a wide variety of dosage forms based on further processing of the powders. These include, but are not limited to, suspensions in liquids, gels or creams, filled capsules, pessaries, gel/polymer matrices and tablets.

For instance, the powders can be suspended in physiologically acceptable solutions for administration by inhalation. In some embodiments, the powders may be in the form of microspheres. In some embodiments, the compositions can contain other ingredients conventional in pharmaceutical compositions including, but not limited to, flavorants, perfumes, hormones such as estrogen, Vitamins such as A, C or E, alpha-hydroxy or alpha-keto acids such as pyruvic, lactic or glycolic acids, lanolin, vaseline, aloe vera, methyl or propyl paraben, pigments and the like.

In one method of making the compositions, the pharmaceutical agent and HDC(s) (and, optionally, surfactant) are mixed, melted to form a homogeneous mix that is then rapidly quenched to a glass incorporating the pharmaceutical agent and the surfactant (if added). The HDC melts are excellent solvents for many organic molecules. This makes them particularly suitable for use in delivery of pharmaceutical materials otherwise difficult to formulate. More than 20% weight percent of organic molecules can be incorporated into the compositions. Notably, HDCs are inert and show no reactivity to their solutes or bioactive agents incorporated therein.

In another method of making the compositions, the HDCs and iloprost and/or another pharmaceutical agent to be administered in addition to iloprost (and, optionally, surfactants) are dissolved in at least one solvent therefor and the glass incorporating the bioactive agent (and surfactant) is formed by evaporation of the solvent. Suitable solvents include, but are not limited to, dichloromethane, chloroform, dimethylsulfoxide, dimethylormamide, acetone, ethanol, propanol and the higher alcohols. The nature of the solvent is immaterial as it is removed in the formation of the delivery system. On evaporating the solvent, the HDCs concentrate to form a glass incorporating the iloprost and/or another pharmaceutical agent to be administered in addition to iloprost with properties similar to the glass formed by quenching from the melt.

Other methods of making the compositions include, but are not limited to, spray drying, freeze drying, air drying, vacuum drying, fluidized-bed drying, milling, co-precipitation and super-critical fluid evaporation. In these methods, the HDC, iloprost and/or another pharmaceutical agent to be administered in addition to iloprost and any other components are first dissolved or suspended in suitable solvents. In the case of milling, glasses formed from the components, either by solvent evaporation or quenching of the melt, are milled in the dried form and processed by any method known in the art. In the case of co-precipitation, the components are mixed in organic conditions and processed as described above.

In the case of spray drying, the components are mixed under suitable solvent conditions and dried using precision nozzles to produce extremely uniform droplets in a drying chamber. Suitable spray drying machines include, but are not limited to, Buchi, NIRO, APV and Lab-plant spray dryers used according to the manufacturer's instructions.

In some embodiments, the iloprost and/or another pharmaceutical agent to be administered in addition to iloprost may be administered using the methods and compositions described in U.S. Pat. No. 6,352,722, the disclosure of which is incorporated herein by reference in its entirety. For example, in some embodiments the iloprost and/or another pharmaceutical agent to be administered in addition to iloprost is provided in compositions comprising derivatized carbohydrates. The derivatized carbohydrates are generally polyol carbohydrates, wherein at least a portion of the hydroxyl groups on the carbohydrate are substituted with a branched hydrophobic chain, such as a hydrocarbon chain, via, for example, an ether or ester linkage. The derivatized carbohydrates are in one embodiment oligosaccharide ester derivatives, such as ester derivatives of disaccharides.

The derivatized carbohydrates can be formed by modification of carbohydrates. Suitable carbohydrates include, but are not limited to, glucose, lactose, cellobiose, sucrose, trehalose, raffinose, melezitose and stachyose. The hydroxyl groups of the carbohydrate can be substituted, for example via ester or ether linkages, with a branched hydrocarbon chain, such as a C3 to C30 branched hydrocarbon chain. The branched hydrocarbon chain can be a C3 to C30 hydrocarbon chain, for example, a C3 to about a C20 hydrocarbon chain. In a preferred embodiment, the hydrocarbon chain includes about a C3 to C8 hydrocarbon chain. The carbohydrate can be substituted, for example, by esterification of one or more of the hydroxyl groups on the carbohydrate with an acid such as a fatty acid including a branched hydrocarbon chain. Mixed esters and ethers of acids including a branched hydrocarbon chain can be formed, e.g., isobutyrate, pivalate, 2,2-dimethylbutyrate, 3,3-dimethylbutyrate, and 2-ethyl butyrate. Optionally, one or more of the remaining hydroxyl groups can be substituted via an ester bond with an acid such as acetate, propionate, or butyrate.

In one embodiment, the substituted carbohydrate can be substituted trehalose (Formula 4) substituted sucrose (Formula 5), substituted lactose (Formula 6), or substituted cellobiose (Formula 7), as shown below. Both α and β anomers and mixtures thereof are encompassed by the invention.

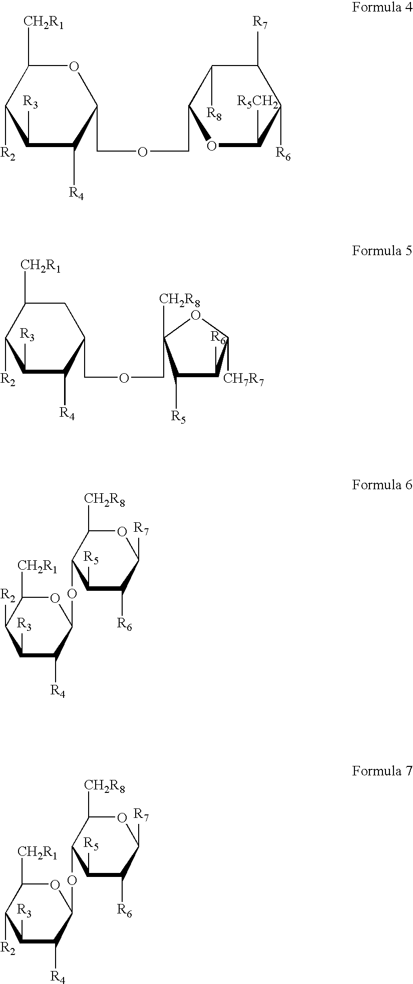

In each of Formulas 4-7, one or more of R₁₋₈ independently NHR₉, N(R₉)₂, O(C═O)R₉, or OR₉, wherein R₉ is a branched, saturated or unsaturated, C3-C20 hydrocarbon, e.g., a C3-C8 hydrocarbon, and preferably a C5-C6 hydrocarbon. O(C═O)R₉ can be, for example, an acid acyl group of an acid such as isobutyrate, pivalate, 2,2-dimethylbutyrate, 3,3-dimethylbutyrate, 2-ethyl butyrate. In each of Formula 4-7, the remainder of R₁₋₈ are independently OH, NHR₁₀, N(R₁₀)₂, O(C═O)R₁₀, or OR₁₀, wherein R₁₀ is alkyl, for example a C1-C4 alkyl group, such as methyl, butyl, or propyl.

Preferred derivatized carbohydrates include trehalose hexa-3,3-dimethylbutyrate, trehalose diacetate-hexa-3,3-dimethylbutyrate, trehalose octa-3,3-dimethylbutyrate, lactose isobutyrate-heptaacetate, lactose 3-acetyl-hepta-3,3-dimethylbutyrate and lactose octa-3,3-dimethylbutyrate.

Derivatized carbohydrates within the scope of the invention further include carbohydrates, such as disaccharides, wherein one or more of the free hydroxyl groups are derivatized, for example into an amine or sulfur group, to which hydrophobic branched hydrocarbon chains can be attached, for example, via an amide or thiol linkage.

Compositions, such as delivery systems, comprising the derivatized carbohydrates, and other components such as pharmaceutical agents, carbohydrates, lipids, phospholipids, surfactants, binders, and any other constituents suitable for use in drug delivery are also encompassed by the invention. The pharmaceutical agents, carbohydrates, lipids, phospholipids, surfactants, binders, and any other constituents suitable for use in drug delivery may be any of those described throughout the present application and may be present in any of the amounts described throughout the present specification. The compositions can be in a vitreous or crystalline form, or mixtures thereof.

Solid dose delivery systems including a substituted carbohydrate can have incorporated therein iloprost and/or another pharmaceutical agent to be administered in addition to iloprost such that these pharmaceutical agents can be released from the solid delivery system. In a preferred embodiment, the solid dose delivery system comprises the substituted carbohydrate in the form of a vitreous glass matrix having the iloprost and/or another pharmaceutical agent to be administered in addition to iloprost. Advantageously, the iloprost and/or another pharmaceutical agent to be administered in addition to iloprost are thereby provided in a solid, non-hygroscopic, glassy matrix, which undergoes a controlled, surface-led devitrification when immersed in aqueous environments and subsequently effects a sustained release of the pharmaceutical agents therein.

Properties of the glassy matrix, such as the release rate of the substance, can be modulated by choice of modified carbohydrate, and other incorporated materials. The glass matrix can be modified, for example, by the addition of different glass formers with known release rates. Other materials can be incorporated into the glass matrix during processing to modify the properties of the final composition, including physiologically acceptable glass formers such as carboxylate, nitrate, sulfate, bisulfate, and combinations thereof. The delivery systems can further incorporate any other suitable carbohydrate and/or hydrophobic carbohydrate derivative, such as glucose pentaacetate or trehalose octaatacetate.

The delivery systems can be in any of a variety of forms including a, microparticle, microsphere, or powder.

The invention further encompasses methods of making the delivery systems. In one embodiment, the method comprises forming or obtaining a substituted carbohydrate capable of forming a vitreous glass; processing the substituted carbohydrate and the iloprost and/or another pharmaceutical agent to be administered in addition to iloprost to be released therefrom; and forming a solid matrix having the iloprost and/or another pharmaceutical agent to be administered in addition to iloprost incorporated therein.

The processing step can be implemented by melting the substituted carbohydrate and incorporating the iloprost and/or another pharmaceutical agent to be administered in addition to iloprost in the melt, at a temperature sufficient to fluidize the substituted carbohydrate, and insufficient to substantially inactivate the iloprost and/or another pharmaceutical agent to be administered in addition to iloprost, and then quenching the melt. The melt can be processed into a variety of forms. The processing step can be also implemented by dissolving or suspending the substituted carbohydrate and the iloprost and/or another pharmaceutical agent to be administered in addition to iloprost in a solvent effective in dissolving at least one of the derivatized carbohydrates and the iloprost and/or another pharmaceutical agent to be administered in addition to iloprost, and evaporating the solvent.

The invention also encompasses methods of delivering iloprost and/or another pharmaceutical agent to be administered in addition to iloprost by providing the delivery systems described above and administering the system to a biological tissue. Administration can be by any suitable means including mucosal, by-inhalation, or any other desired route.

In some embodiments, the delivery systems may be used to deliver hydrophobic substances. The invention encompasses these delivery systems.

To improve the glass-forming characteristics of such hydrophobically derivatized carbohydrates, in some embodiments, the carbon chains longer than 4 carbons in a branched chain may be used to provide hydrophobically derivatized carbohydrates that form suitable glasses, both vitreous and crystalline, for use to formulate iloprost and/or another pharmaceutical agent to be administered in addition to iloprost, and facilitate their controlled delivery, enabling their use in solid dose delivery systems.

Derivatized carbohydrates are provided, as well as compositions comprised thereof and methods of use thereof. The derivatized carbohydrates are generally carbohydrates wherein at least a portion of the hydroxyl groups on the carbohydrate are substituted with a branched hydrophobic chain, such as a hydrocarbon chain, via, for example, an ether or ester linkage. The derivatized carbohydrates can be formed by modification of carbohydrates, including, but not limited to, glucose, lactose, cellobiose, sucrose, trehalose, raffinose, melezitose and stachyose. The hydroxyl groups of the carbohydrate can be substituted, for example via ester or ether linkages, with a branched hydrocarbon chain, for example a C3 to about a C20 hydrocarbon chain. In a preferred embodiment, the hydrocarbon chain is about a C3 to C8 hydrocarbon chain. Preferred derivatized carbohydrates include trehalose hexa-3,3-dimethylbutyrate; trehalose diacetate-hexa-3,3-dimethylbutyrate; trehalose octa-3,3-dimethylbutyrate; lactose octa-3,3-dimethylbutyrate; lactose 3-acetyl-hepta-3,3-dimethylbutyrate; and lactose isobutyrate-heptaacetate.

The derivatized carbohydrates are particularly useful in forming solid vehicles, such as vitreous glass matrices. The solid vehicles, such as vitreous glasses, can be processed into different solid forms, including tablets, powders, lozenges, implants and microspheres. In some embodiments, the solid matrices are useful as biodegradable solid materials for controlled delivery and release of incorporated iloprost and/or another pharmaceutical agent to be administered in addition to iloprost.

Formation of Derivatized Carbohydrates

The derivatized carbohydrates are formed in one embodiment by the esterification of the free hydroxyl groups on a carbohydrate. Additional other methods known in the art can be used such as etherification of the free hydroxyls. In one embodiment, at least a portion of the free hydroxyl groups are esterified with a branched hydrocarbon chain acid, or mixtures thereof. Additionally, optionally, all or a portion of the remainder of the free hydroxyls are esterified with another acid, such as alkyl acids, e.g., acetic acid, propionic acid, butyric acid, or mixtures thereof. A wide variety of partial and mixed esters can be formed. Suitable acids for ester formation with free hydroxyls on the carbohydrate that include a branched hydrocarbon chain include isobutyrate, pivalate, 2,2-dimethylbutyrate, 3,3-dimethylbutyrate, and 2-ethyl butyrate.

Carbohydrates which can be substituted at the hydroxyl group include disaccharides such as trehalose, sucrose, lactose and cellobiose, the structures of which are shown below. Either pure anomers or anomer mixtures can be used.

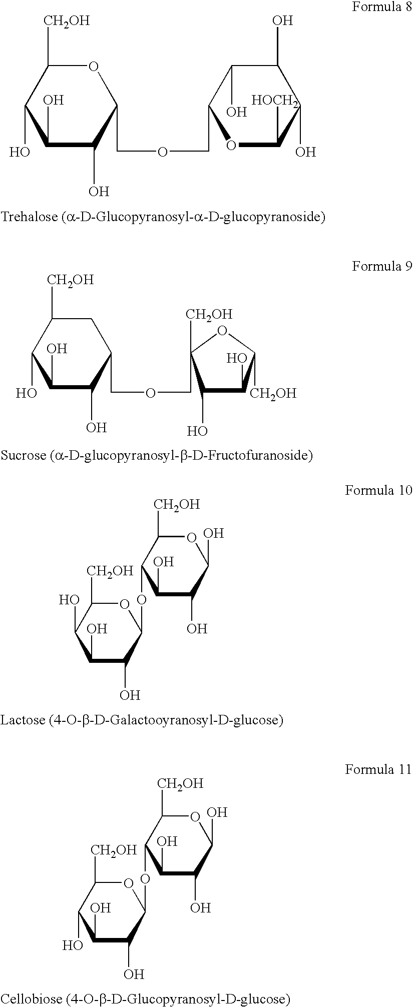

Methods for esterifying the carbohydrates are available in the art. For example, the carbohydrates can be treated with dimethylbutyroyl chloride in anhydrous pyridine to form the dimethylbutyroylated carbohydrate. Additionally, partial or mixed esters can be formed by manipulation of the reaction conditions and reagent amounts. Such partial and/or mixed esters are also encompassed by the invention.

The invention encompasses a variety of derivatized carbohydrates. Preferred derivatized carbohydrates include trehalose hexa-3,3-dimethylbutyrate, trehalose diisobutyrate-hexaacetate, trehalose diacetate-hexa-3,3-dimethylbutyrate, trehalose octa-3,3-dimethylbutyrate and lactose isobutyrate-heptaacetate.

The reaction product can be characterized structurally by methods known in the art, including, but not limited to, nuclear magnetic resonance spectroscopy (NMR) and its material science properties characterized by differential scanning calorimetry (DSC). The characteristic melting points and Tgs (glass transition temperatures) for the derivatized carbohydrates can also be determined by DSC and other methods known in the art.

Properties of Derivatized Carbohydrates

Many carbohydrates fail to readily crystallize when dried from solvent. In the absence of crystal growth, an alternative solid state, that of an amorphous, optically transparent vitreous glass is formed. A thermodynamic transition (Tg), measured by calorimetry, is characteristic of the viscous state and defines the temperature range over which the highly viscous state collapses into a more fluid rubbery state. Eventually, as the temperature continues to rise, the viscosity will fall further, resulting in a liquid melt.

In the usual process to form a vitreous glass, a high temperature melt is quenched (cooled quickly) to solidify without crystallization to a vitreous glass. Most glassy materials can theoretically quench to a vitreous glass, however, factors such as low melt viscosity, thermodynamically favorable crystalline states and thermal degradation, limit their potential to form vitreous rather than crystalline solids.

The glass matrices formed from derivatized carbohydrates as described herein can be used to stabilize labile bioactive molecules immobilized within the glassy matrix, both crystalline and vitreous. Preferably, the glassy state is vitreous. Preferred derivatized carbohydrates have high Tgs in the vitreous form, e.g., about 40° C. to 85° C., and are physically stable. The vitreous glass matrices formed therefrom have increased hydrophobicity, and thus have many applications as drug delivery vehicles, particularly for administration as sustained or delayed release forms. The derivatized carbohydrates permit solid matrices to be formed therefrom with selected controlled release properties. Without being limited to any one theory, it is believed that when the solid amorphous matrix is immersed in aqueous environments, drug release is effected by a controlled devitrification or crystallization, which begins over the surface of the glass particle. As water interacts with the glass, the devitrification front proceeds further into the glass. The crystalline matrix thus formed allows the previously entrapped drug to diffuse into the surrounding environment at a rate dependent on both HDC and drug.

The invention enables the preparation and use of derivatized carbohydrates having glass transition temperatures (Tgs) high enough to form stable glasses to allow the formulation of actives such as drugs. In parallel, the glasses undergo a slow, controlled devitrification when immersed in water. The methods of the invention permit the formulation of iloprost and/or another pharmaceutical agent to be administered in addition to iloprost in very hydrophobic glassy matrices, which can sustain release of these pharmaceutical agents over long time periods.

Derivatized carbohydrates can also be used to form solid matrices that have a partially or substantially crystalline structure. Additionally, glasses can also be formed which form a partially or substantially crystalline structure over time after incorporation of active.

Using the methods disclosed herein, in one embodiment, C₅ and C₆ branched chain fatty acid derivatives of trehalose, and other carbohydrate molecules such as lactose, cellobiose, sucrose, raffinose and stachyose can be made, which can be melted and quenched to glasses with higher Tgs, e.g., greater than about 30° C., preferably greater than about 40° C.

The Tgs of the vitreous forms of the compositions encompassed herein are typically less than about 200° C., typically about 10° C. to 100° C., preferably about 20° C. and 85° C. The derivatized carbohydrates can be used to form vitreous glass matrices, wherein the tendency to crystallize from the melt or with reducing solvent, is low. Mixtures of derivatized carbohydrates also can be used to form the glass matrices. Glasses formed using the derivatized carbohydrates preferably have melt temperatures suitable for the incorporation of substances such as biologically active compounds, without thermal degradation, and have Tgs above ambient temperatures.

Both devitrification of the vitreous matrix and the fluidity of the melt at temperatures close to Tg can be controlled by choice of the degree and type of substitution of the carbohydrate, and by the addition of modifiers such as other derivative sugars and certain organic compounds. Suitable derivative sugars and organic compounds are described for instance, in PCT GB95/01861, the disclosure of which is incorporated herein by reference in its entirety.

As used herein, ambient temperatures are those of the surrounding environment of any given environment. Typically, ambient temperatures are “room temperature” which is generally 20-22° C. However, ambient temperature of a “warm room” (for bacteriological growth) can be 37° C. Thus, ambient temperature is readily determined from the context in which it is used and is well understood by those of skill in the art.

Formation of Delivery Systems

The derivatized carbohydrates provided herein can be used to form a biodegradable delivery system with iloprost and/or another pharmaceutical agent to be administered in addition to iloprost incorporated therein. The derivatized carbohydrates are referred to herein as the “vehicle” used to form the delivery system. As used herein, the term “delivery system” refers to any form of the substituted carbohydrate having iloprost and/or another pharmaceutical agent to be administered in addition to iloprost incorporated therein. Preferably, the delivery system is in the form of a solid matrix having the iloprost and/or another pharmaceutical agent to be administered in addition to iloprost incorporated therein. The matrix can be designed to have a desired release rate of the iloprost and/or another pharmaceutical agent to be administered in addition to iloprost incorporated therein, by selection of the material forming the matrix, selection of the conditions of forming the matrix, and by the addition of other substances which can modify the rate of release.

The derivatized carbohydrates readily form glasses either from a quenched melt or an evaporated organic solvent. Examples of methods of forming amorphous carbohydrate glass matrices are described in “Pharmaceutical Dosage Forms,” Vol. 1 (H. Lieberman and L. Lachman, Eds.) 1982, the disclosure of which is incorporated herein by reference in its entirety.

The derivatized carbohydrates and iloprost and/or another pharmaceutical agent to be administered in addition to iloprost to be incorporated can be intimately mixed together in the appropriate molar ratios and melted until clear. Suitable melting conditions include, but are not limited to, melting in open glass flasks at about 30-250° C. for about 1-2 minutes. This results in a fluid melt which can be allowed to cool slightly before dissolving the substance in the melt, if required, and quenching to glass for instance by pouring over a brass plate or into a metal mould for shaped delivery vehicles. The melts can also be quenched by any methods including spray chilling. Melt temperature can be carefully controlled and iloprost and/or another pharmaceutical agent to be administered in addition to iloprost can be incorporated into the derivatized carbohydrates either in the pre-melted formulation, or stirred into the cooling melt before quenching.

The melts are thermally stable and allow the incorporation of molecules without denaturation, or suspension of core particles without alteration of their physical nature. The glass melts can be used also to coat micron-sized particles. This is particularly important in the formulation of non-hygroscopic powders containing hygroscopic actives for by-inhalation administration of therapeutic agents. Compositions made by this process are also encompassed by this invention.

Alternatively, delivery systems can be formed by evaporation of the derivatized carbohydrates and iloprost and/or another pharmaceutical agent to be administered in addition to iloprost to be incorporated in solution in a solvent or mixture thereof. Suitable organic solvents include, but are not limited to, dichloromethane, chloroform, dimethylsulfoxide, dimethylformamide, ethyl acetate, acetone and alcohols. The type of solvent is immaterial as it is completely removed on formation of the delivery system. Preferably, both the substituted carbohydrate and substance to be incorporated are soluble in the solvent. However, the solvent can dissolve the substituted carbohydrate and allow a suspension of the iloprost and/or another pharmaceutical agent to be administered in addition to iloprost to be incorporated in the matrix. In one embodiment, on concentrating the solvent, crystallization of the derivatized carbohydrates does not occur. Instead, a vitreous solid is produced, which has similar properties to the quenched glass. Alternatively, solid matrices which are partially, substantially or fully crystalline can be formed iloprost and/or another pharmaceutical agent to be administered in addition to iloprost can be incorporated easily either in solution or as a particle suspension.

In one embodiment, a solution of the iloprost and/or another pharmaceutical agent to be administered in addition to iloprost to be incorporated, containing a sufficient quantity of substituted carbohydrate to form a glass on drying, can be dried by any method known in the art, including, but not limited to, freeze drying, lyophilization, vacuum, spray, belt, air or fluidized-bed drying. Another suitable method of drying, exposing a syrup to a vacuum under ambient temperature, is described in PCT GB96/01367, the disclosure of which is incorporated herein by reference in its entirety. After formation of a glass containing homogeneously distributed iloprost and/or another pharmaceutical agent to be administered in addition to iloprost in solid solution or fine suspension in the glass, the glasses can then be milled and/or micronized to give microparticles of homogeneous defined size.

Different dosing schemes can also be achieved by the delivery system formulated. The delivery system can permit a quick release or flooding dose of the incorporated iloprost and/or another pharmaceutical agent to be administered in addition to iloprost after administration, upon dissolving and release of the iloprost and/or another pharmaceutical agent to be administered in addition to iloprost from the delivery system. Coformulations of vehicles with slowly water-soluble glasses and plastics such as phosphate, nitrate or carboxylate glasses and lactide/glycolide, glucuronide or polyhydroxybutyrate plastics and polyesters, provide more slowly dissolving vehicles for a slower release and prolonged dosing effect. Optionally, iloprost and/or another pharmaceutical agent to be administered in addition to iloprost can be incorporated into the vitreous matrix which retards recrystallization of the matrix, such as polyvinylpyrolidone, or a hydrophobic substance, to modify the release rate of these pharmaceutical agents, such as a water insoluble wax or a fatty acid. These are described in PCT WO 93/10758, the disclosure of which is incorporated herein by reference in its entirety.

The delivery systems can also be coformulated with a hydrophobically-derivatized carbohydrate (HDC) glass forming material. Suitable HDC glass forming materials include, but are not limited to, those described in PCT WO 96/03978, the disclosure of which is incorporated herein by reference in its entirety. As used herein, HDC refers to a wide variety of hydrophobically derivatized carbohydrates where at least one hydroxyl group is substituted with a hydrophobic moiety. Examples of suitable HDCs and their syntheses are described in Developments in Food Carbohydrate—2 ed. C. K. Lee, Applied Science Publishers, London (1980), the disclosure of which is incorporated herein by reference in its entirety. Other syntheses are described for instance, in Akoh et al. (1987) J. Food Sci. 52:1570; Khan et al. (1993) Tet. Letts 34:7767; Khan (1984) Pure & Appl. Chem. 56:833-844; and Khan et al. (1990) Carb. Res. 198:275-283, the disclosures of which are incorporated herein by reference in their entireties.

The delivery of more than one pharmaceutical agent can also be achieved using a delivery system including multiple coatings or layers loaded with different materials or mixtures thereof. Administration of the solid dose delivery systems of the present invention can be used in conjunction with other conventional therapies and coadministered with other therapeutic, prophylactic or diagnostic substances. Compositions such as these are encompassed by the invention.

The solid delivery systems can be used to deliver therapeutic agents by any means including, but not limited to, transmucosal and by-inhalation (naso-pharyngeal and pulmonary, including transbronchial and transalveolar).

The delivery systems suitable for transmucosal delivery include, but are not limited to powders.

Compositions suitable for by-inhalation administration include, but are not limited to, powder forms of the delivery systems. There are a variety of devices suitable for use in by-inhalation delivery of powders. See, e.g., Lindberg (1993) Summary of Lecture at Management Forum Dec. 6-7, 1993 “Creating the Future for Portable Inhalers”, the disclosure of which is incorporated herein by reference in its entirety. Additional devices suitable for use herein include, but are not limited to, those described in WO9413271, WO9408552, WO9309832 and U.S. Pat. No. 5,239,993, the disclosures of which are incorporated herein by reference in their entireties.

The delivery systems are preferably biodegradable and release iloprost and/or another pharmaceutical agent to be administered in addition to iloprost incorporated therein over a desired time period, depending on the particular application, and the composition of the system. As used herein, the term “biodegradable” refers to the ability to degrade under the appropriate conditions of use, such as outdoors, or in the body, for example by dissolution, devitrification, hydrolysis or enzymatic reaction.

Substances Incorporated in the Delivery Systems

Iloprost and/or any of the other pharmaceutical agents to be administered in addition to iloprost described herein may be administered using the disclosed compositions.

In some embodiments of the present invention, the iloprost and/or another pharmaceutical agent to be administered in addition to iloprost are administered using the methods and compositions described in U.S. patent application Ser. No. 09/923,023 (published as US 2002/0009464), the disclosure of which is incorporated herein by reference in its entirety. In some embodiments, the iloprost and/or another pharmaceutical agent to be administered in addition to iloprost are provided in formulations comprising modified glycosides. The modified glycosides include lactitol nonaacetate, palatinit nonaacetate, glucopyranosyl sorbitol nonaacetate, glucopyranosyl mannitol nonaacetate and mixtures thereof. The modified glycosides can be formed by modification of polyol glycosides such as lactitol (4-O-β-D-galactopyranosyl-D-glucitol), palatinit [αmixture of GPS (α-D-glucopyranosyl-1→6-sorbitol) and GPM (α-D-glucopyranosyl-1→6-mannitol)], the individual glycoside components thereof, GPS and GPM, maltitol (4-O-β-D-glucopyranosyl-D-glucitol), hydrogenated maltooligosaccharides (such as maltotritol, maltotetraitol, maltopentaitol, maltohexaitol, maltooctaitol, maltononaitol and maltodecaitol) and hydrogenated isomaltooligosaccharides. The modified glycosides may be, for example, ester or ether derivatives of glycosides, or mixed ester or ether derivatives of glycosides. The modified glycosides can include saccharide and oligosaccharide subunits, such as furanose or pyranose saccharide subunits, or mixtures thereof.

Exemplary structures of modified glycosides are shown below:

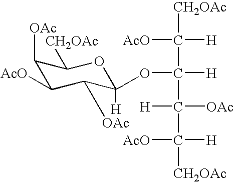

### lactitol nonaacetate (4-O-β-D-galactopyranosyl-D-glucitol nonaacetate)

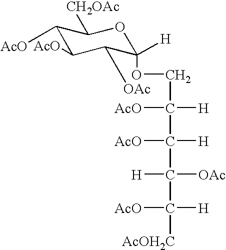

### GPS nonaacetate (α-D-glucopyranosyl-1→6-sorbitol nonaacetate)

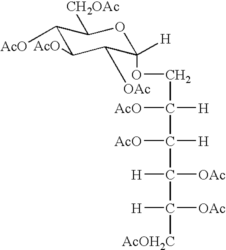

### GPM nonaacetate (α-D-glucopyranosyl-1→6-mannitol nonaacetate)

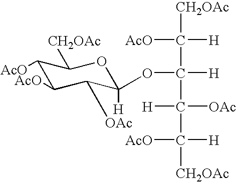

### maltitol nonaacetate (4-O-θ-D-glucopyranosyl-D-glucitol nonaacetate)

In one embodiment, the modified glycosides are represented by Formula 12 or Formula 13 shown below.

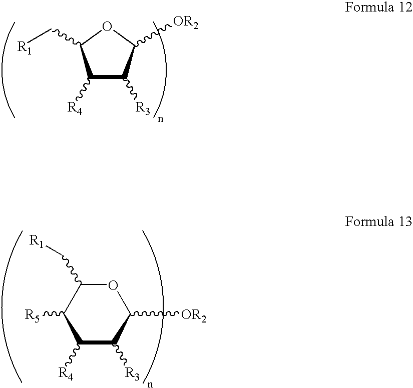

wherein R₁, R₃, R₄ and R₅ are independently OH, NH₂, NHR₆, N(R₆)₂, OR₆ or O(C═O)R₆, wherein R₆ is alkyl, preferably a straight chain or branched, saturated or unsaturated, C1-C25 hydrocarbon, such as a C1-C15 hydrocarbon, or in one preferred embodiment, a C1-C8 hydrocarbon, for example, methyl or isobutyl;

wherein OR₂ is a monosaccharide polyalcohol, preferably a reduced monosaccharide 5 or 6 carbon polyalcohol, such as ribitol, xylitol, mannitol or glucitol; and

wherein n is 1-6, where each subunit, n, may include the same or different substituents, R₁, R₃, R₄ and R₅ and wherein the subunits are linked in a linear or branched chain via a C N or O linkage at the positions R₁, R₃, R₄ or R₅.

Modified glycosides within the scope of the invention include modified glycosides of sugar alcohols, also referred to herein as hydrogenated oligosaccharides. The modified glycosides are in one embodiment derivatives of hydrogenated maltooligosaccharides or derivatives of hydrogenated isomaltooligosaccharides. As used herein, the “hydrogenated oligosaccharide” refers to an oligosaccharide including preferably about 2 to 7 saccharide units, wherein a terminal saccharide subunit is reduced and is in the form of a polyalcohol. As used herein, the term “hydrogenated maltooligosaccharide” refers to a branched or straight chain oligosaccharide including about 2 to 7 glucose units, linked by glycoside linkage, wherein a terminal glucose subunit is reduced and is in the form of the polyalcohol, glucitol. As used herein, the term “hydrogenated isomaltooligosaccharide” refers to a branched oligosaccharide including about 2 to 7 glucose units, linked by glycoside linkage, wherein a terminal fructose subunit is reduced and is in the form of the polyalcohols sorbitol or mannitol.

The modified glycosides in one embodiment are glycosides which are derivatized to render them hydrophobic, for example, by the esterification of at least a portion of free hydroxyl groups on the glycoside with fatty acid acyl groups. The modified glycoside in one embodiment is a hydrophobic ester or mixed ester derivative of a glycoside of a sugar alcohol. In one preferred embodiment, the modified glycoside is a hydrophobic derivative of a hydrogenated maltooligosaccharide, which is rendered hydrophobic by derivatization of the free hydroxyl groups, for example, to form fatty acid acyl esters or long hydrocarbon chain ethers. In one embodiment, the modified glycosides are represented by compounds of Formula 14 below:
(Y)ₙ—X Formula 14

where Y represents a saccharide subunit, or derivative thereof, and n is 1-6, wherein each of the n saccharide subunits are linked in a linear or branched chain by glycosidic linkages; and where X is a 5 or 6 carbon monosaccharide polyalcohol, such as ribitol, xylitol, mannitol or glucitol. For example, (Y)n may be a branched or straight chain oligosaccharide including glucose subunits which are linked by an α- or β-glucosidic linkage, such as a 1→6 or 1→4 linkage, and X can be a polyalcohol linked via a glycosidic bond to an anomeric carbon on one of the glucose subunits. In the compounds of Formula 14, all or a portion of the free hydroxyl groups in the saccharide subunits and the polyalcohol are derivatized in the form of esters, ethers, mixed esters or mixed ethers. For example, the free hydroxyl groups may be reacted with the appropriate reagent to form acyl esters, isobutyl esters, or esters of C1-C25 saturated or unsaturated branched or straight chain fatty acids, or mixtures thereof. The modification of the hydroxyl groups with the ester or ether functionalities thus can render the compound hydrophobic. An exemplary compound is shown below:

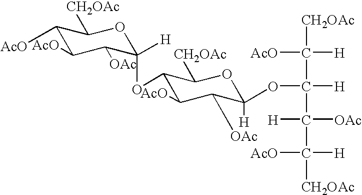

### 4-O-(α-D-glucopyranosyv)4-O-(β-D-glucopyranosyl)-D-glucitol dodecaacetate.

Compositions comprising the modified glycosides, and other components such as bioactives, carbohydrates, binders, surfactants, stabilizing polyols and any other constituent suitable for use in drug delivery are also encompassed by the invention. The bioactives, carbohydrates, binders, surfactants, stabilizing polyols and other constituents suitable for use in drug delivery may be any of those described throughout the present application and may be present in any of the amounts described throughout the present application, including those discussed with respect to the hydrophobically derivatized carbohydrates (HDCs) discussed above.

Solid delivery systems are provided, which comprise a modified glycoside having incorporated therein a substance capable of being released from the solid delivery system. The release rate of the iloprost and/or another pharmaceutical agent to be administered in addition to iloprost can be modulated by the addition of different glass formers with known release rates. In a preferred embodiment, the solid delivery system comprises the modified glycoside in the form of a vitreous glass matrix having the iloprost and/or another pharmaceutical agent to be administered in addition to iloprost incorporated therein. In one preferred embodiment, the modified glycoside in the solid delivery systems is an acetylated glycoside. Preferred modified glycosides are lactitol nonaacetate, palatinit nonaacetate, glucopyranosyl sorbitol nonaacetate, glucopyranosyl mannitol nonaacetate or maltitol nonaacetate.

The invention further encompasses compositions comprising a modified glycoside and a second physiologically acceptable glass material, such as a carboxylate, nitrate, sulfate, bisulfate, or combinations thereof. The delivery systems can further incorporate any other carbohydrate and/or hydrophobic carbohydrate derivative (HDC), such as trehalose octaacetate.

The solid delivery systems can be in any of a variety of forms including a, microparticle, microsphere, or powder.

The invention further encompasses methods of making the solid delivery systems. In one embodiment, the method comprises forming a modified glycoside capable of forming a vitreous glass; processing the modified glycoside and iloprost and/or another pharmaceutical agent to be administered in addition to iloprost to be released therefrom, and forming a vitreous glass matrix having the iloprost and/or another pharmaceutical agent to be administered in addition to iloprost incorporated therein.

The processing step can be implemented by melting the modified glycoside and incorporating the iloprost and/or another pharmaceutical agent to be administered in addition to iloprost in the melt, at a melt temperature sufficient to fluidize the modified glycoside, and insufficient to substantially inactivate the substance, and then quenching the melt. The melt can be processed into a variety of forms. The processing step can be further implemented by dissolving or suspending the modified glycoside and the iloprost and/or another pharmaceutical agent to be administered in addition to iloprost in a solvent effective in dissolving at least one of the modified glycosides and the iloprost and/or another pharmaceutical agent to be administered in addition to iloprost, and evaporating the solvent.

Methods of making the modified glycosides are also provided. The modified glycoside can be provided in one embodiment by acetylating free hydroxyl groups on a glycoside, to form the modified glycoside. In one embodiment, lactitol, palatinit, glycopyranosyl sorbitol or glycopyranosyl mannitol are acetylated to form the modified glycosides, lactitol nonaacetate, palatinit nonaacetate, glucopyranosyl sorbitol nonaacetate, and glucopyranosyl mannitol nonaacetate, respectively.

The invention further encompasses glass matrices comprising the modified glycosides. The qualities of the glass matrices can be modified by choice of modified carbohydrate, and other incorporated materials, to have a desired rate of release of the incorporated iloprost and/or another pharmaceutical agent to be administered in addition to iloprost. Other materials can be incorporated into the glass matrix during processing to modify the properties of the final composition, including physiologically acceptable glasses such as carboxylate, nitrate, sulfate, bisulfate, and combinations thereof.

The invention also encompasses methods of delivering bioactive materials by providing the solid dose delivery systems described above and administering the system to a biological tissue. Administration can be by any suitable means including mucosal and by-inhalation.

In some embodiments, the delivery systems are utilized to deliver hydrophobic pharmaceutical agents.

In some embodiments, the iloprost and/or another pharmaceutical agent to be administered in addition to iloprost are provided in a formulation comprising modified glycosides of sugar alcohols, which are particularly useful in forming vitreous glass matrices. The modifications include ester and ether derivatives in either single or mixed compositions. A wide variety of pharmaceutical agents can be incorporated into the glass matrices.

The modified glycosides are formed in one embodiment by the esterification of the free hydroxyl groups on a glycoside. Preferred modified glycosides within the scope of the invention include, but are not limited to, lactitol nonaacetate, palatinit nonaacetate, glucopyranosyl sorbitol nonaacetate, and glucopyranosyl mannitol nonaacetate. The modified glycosides are useful in forming vitreous glasses which can be processed into different solid forms, including tablets, powders, lozenges, implants and microspheres.

The use of gel-sol techniques for the formation of glassy matrices has enabled applications such as monoliths, fibers, coating, films, etc. “Glasses and Glass Ceramics From Gels,” Ed., S. Sakka. (1987) North-Holland, Amsterdam, the disclosure of which is incorporated herein by reference in its entirety. These applications can now be extended using techniques of formation of glassy matrices by solvent evaporation, and/or from the melt, if organic glass formers are used. Particular advantages of the group of novel organic glass forming modified glycosides described herein is their low cost, biodegradability, ease of synthesis and good solvent properties for various actives including organic molecules such as bioactives and optical actives (e.g. dyes and photochromes) and even inorganic compounds such as mixed transition metal oxides and metal alkoxides.

Formation of Modified Glycosides

The modified glycosides are formed in one embodiment by the esterification of the free hydroxyl groups on a glycoside. For example, all of the free hydroxyl groups can be esterified with acetic acid or propionic acid, or mixtures thereof. Alternatively, partial or mixed esters can be formed.

Methods for esterifying the glycosides are available in the art. For example, the glycosides can be treated with sodium acetate in acetic anhydride to form the acetylated polyol. Additionally, partial or mixed esters can be formed by manipulation of the reaction conditions and reagent amounts. Such partial and/or mixed esters are also encompassed by the invention.

A variety of modified glycosides are within the scope of the invention. For example, polyol glycosides of sugar alcohols may be esterified with acetyl groups. In a preferred embodiment, the polyols are lactitol (4-O-β-D-galactopyranosyl-D-glucitol), palatinit [a mixture of GPS (α-D-glucopyranosyl-1→6-sorbitol) and GPM (α-D-glucopyranosyl-1→6-mannitol)], and the individual glycoside components thereof, GPS (also referred to herein as glucopyranosyl sorbitol) and GPM (also referred to herein as glucopyranosyl mannitol). Additionally, the polyol can be maltitol (4-O-β-D-glucopyranosyl-D-glucitol), or hydrogenated maltooligosaccharides and isomaltooligosaccharides.

In one embodiment, the glycoside is esterified and treated with sodium acetate and acetic anhydride. Examples of this include, but are not limited to, esterification of lactitol, palatinit, GPS or GPM treated with sodium acetate and acetic anhydride, to form respectively, lactitol nonaacetate, palatinit nonaacetate, glucopyranosyl sorbitol nonaacetate, and glucopyranosyl mannitol nonaacetate.

The reaction product can be structurally characterized by nuclear magnetic resonance spectroscopy (NMR) and its material science properties characterized by differential scanning calorimetry (DSC). The characteristic melting points and Tgs (glass transition temperatures) for the modified glycosides can also be determined by DSC and other methods known in the art.

The Tgs of the compositions encompassed herein are low, typically less than about 200° C. and, surprisingly, are not predictable from the melt temperatures. In general, the tendency of the glass matrices described herein to crystallize, from the melt or with reducing solvent, is low. Glasses formed using the modified glycosides preferably have melt temperatures suitable for the incorporation of substances such as biologically active compounds, without thermal degradation, and have Tgs above ambient temperatures.

Both devitrification and the fluidity of the melt at temperatures close to Tg, can be controlled by modifiers such as other derivative sugars and certain organic compounds. Suitable derivative sugars and organic compounds are described for instance, in PCT GB95/01861, the disclosure of which is incorporated herein by reference in its entirety.

As used herein, ambient temperatures are those of the surrounding environment of any given environment. Typically, ambient temperatures are “room temperature” which is generally 20-22° C. However, ambient temperature of a “warm room” (for bacteriological growth) can be 37° C. Thus, ambient temperature is readily determined from the context in which it is used and is well understood by those of skill in the art.

Formation of Delivery Systems

The modified glycosides can be used to form a biodegradable delivery system, optionally with iloprost and/or another pharmaceutical agent to be administered in addition to iloprost incorporated therein. The modified glycosides are referred to herein as the “vehicle” used to form the delivery system. As used herein, the term “delivery system” refers to any form of the modified glycoside having iloprost and/or another pharmaceutical agent to be administered in addition to iloprost incorporated therein. Preferably, the delivery system is in the form of an amorphous, glass-matrix having the iloprost and/or another pharmaceutical agent to be administered in addition to iloprost incorporated therein. The glass matrix advantageously can be designed to have a desired release rate of the iloprost and/or another pharmaceutical agent to be administered in addition to iloprost incorporated therein, by selection of the material forming the matrix, selection of the conditions of forming the matrix, and by the addition of other substances which can modify the rate of release.

The modified glycosides readily form glasses either from a quenched melt or an evaporated organic solvent. Examples of methods of forming amorphous carbohydrate glass matrices are described in “Pharmaceutical Dosage Forms,” Vol. 1 (H. Lieberman and L. Lachman, Eds.) 1982, the disclosure of which is incorporated herein by reference in its entirety.

The modified glycosides in purified form and iloprost and/or another pharmaceutical agent to be administered in addition to iloprost to be incorporated can be intimately mixed together in the appropriate molar ratios and melted until clear. Suitable melting conditions include, but are not limited to, melting in open glass flasks between about 50 and 250° C. for about 1-2 minutes. This results in a fluid melt which can be allowed to slightly cool before dissolving the substance in the melt, if required, and quenching to glass for instance by pouring over a brass plate or into a metal mould for shaped delivery vehicles. Melt temperature can be carefully controlled and iloprost and/or another pharmaceutical agent to be administered in addition to iloprost can be incorporated into the modified glycosides either in the pre-melted formulation, or stirred into the cooling melt before quenching.

The melts are thermally stable and allow the incorporation of molecules without denaturation, or suspension of core particles without alteration of their physical nature. The glass melts can be used also to coat micron-sized particles, this is particularly important in the formulation of non-hygroscopic powders containing hygroscopic actives, for by-inhalation administration of therapeutic agents.

Alternatively, vitreous delivery systems can be formed by evaporation of the modified glycosides and substance to be incorporated in solution in a solvent or mixture of solvents. Suitable organic solvents include, but are not limited to, dichloromethane, chloroform, dimethylsulfoxide (DMSO), dimethylformamide (DMF) and higher alcohols. The exact nature of the solvent is immaterial as it is completely removed on formation of the delivery system. Preferably, both the modified glycoside and iloprost and/or another pharmaceutical agent to be administered in addition to iloprost to be incorporated are soluble in the solvent. However, the solvent may dissolve the modified glycoside and allow a suspension of the iloprost and/or another pharmaceutical agent to be administered in addition to iloprost to be incorporated in the matrix. Preferably, on concentrating the solvent, crystallization of the modified glycosides does not occur. Instead, an amorphous solid (“glass” or “glass matrix” herein) is produced, which has similar properties to the quenched glass. Iloprost and/or another pharmaceutical agent to be administered in addition to iloprost can be incorporated easily either in solution or as a particle suspension.

A solution of the iloprost and/or another pharmaceutical agent to be administered in addition to iloprost to be incorporated containing a sufficient quantity of modified glycoside to form a glass on drying can be dried by any method known in the art, including, but not limited to, freeze drying, vacuum, spray, belt, air or fluidized-bed drying. Another suitable method of drying, exposing a syrup to a vacuum under ambient temperature, is described in PCT GB96/01367, the disclosure of which is incorporated herein by reference in its entirety. After formation of a glass containing homogeneously distributed substance in solid solution or fine suspension in the glass, the glasses can then be milled and/or micronized to give microparticles of homogeneous defined size.

Different dosing schemes can also be achieved by the delivery system formulated. The delivery system can permit a quick release or flooding dose of the incorporated iloprost and/or another pharmaceutical agent to be administered in addition to iloprost after administration, upon the dissolving and release of the iloprost and/or another pharmaceutical agent to be administered in addition to iloprost from the delivery system. Coformulations of vehicles with slowly water soluble glasses and plastics such as phosphate, nitrate or carboxylate glasses and lactide/glycolide, glucuronide or polyhydroxybutyrate plastics and polyesters, provide more slowly dissolving vehicles for a slower release and prolonged dosing effect. Optionally, a substance can be incorporated into the glass matrix which retards recrystallization of the matrix, such as polyvinylpyrrolidone, or a hydrophobic substance can be incorporated in the matrix, so as to modify the release rate of the substance, such as a water insoluble wax or a fatty acid. PCT WO93/10758, the disclosure of which is incorporated herein by reference in its entirety.

The delivery systems can also be coformulated with a hydrophobically-dervatized carbohydrate (HDC) glass forming material. HDC glass forming materials are described in PCT WO96/03978, the disclosure of which is incorporated herein by reference in its entirety. As used herein, HDC refers to a wide variety of hydrophobically derivatized carbohydrates where at least one hydroxyl group is substituted with a hydrophobic moiety. Examples of suitable HDCs and their syntheses are described in Developments in Food Carbohydrate—2 ed., C. K. Lee, Applied Science Publishers, London (1980). Other syntheses are described for instance, in Akoh et al. (1987) J. Food Sci. 52:1570; Khan et al. (1993) Tetra. Letts 34:7767; Khan (1984) Pure & Appl. Chem. 56:833-844; and Khan et al. (1990) Carb. Res. 198:275-283, the disclosures of which are incorporated herein by reference in their entireties.

The delivery of more than one pharmaceutical agent can also be achieved using a delivery system including multiple coatings or layers loaded with different materials or mixtures thereof. Administration of the solid dose delivery systems of the present invention can be used in conjunction with other conventional therapies and coadministered with other therapeutic, prophylactic or diagnostic agents.

The solid delivery systems can be used to deliver therapeutic agents by any means including, but not limited to, transmucosal and by-inhalation (naso-pharyngeal and pulmonary, including transbronchial and transalveolar).

Compositions suitable for by-inhalation administration include, but are not limited to, powder forms of the delivery systems. There are a variety of devices suitable for use in by-inhalation delivery of powders. See, e.g., Lindberg (1993) Summary of Lecture at Management Forum 6-7 Dec. 1993 “Creating the Future for Portable Inhalers.” Additional devices suitable for use herein include, but are not limited to, those described in WO 94/13271, WO 94/08552, WO 93/09832 and U.S. Pat. No. 5,239,993, the disclosures of which are incorporated herein by reference in their entireties.

The delivery systems are preferably biodegradable and release substances incorporated therein over a desired time period, depending on the particular application, and the composition of the system. As used herein, the term “biodegradable” refers to the ability to degrade under the appropriate conditions of use, such as outdoors, or in the body, for example by dissolution, devitrification, hydrolysis or enzymatic reaction.

Substances Incorporated in the Delivery Systems

Iloprost and/or any of the other pharmaceutical agents to be administered in addition to iloprost which are described herein may be administered using the disclosed formulations.

In some embodiments of the glass formulations described herein, the iloprost is present in a concentration between about 0.01%-about 30% by weight. In other embodiments of the glass formulations described herein, the iloprost is present in a concentration between about 0.05%-about 20% by weight. In some embodiments of the glass formulations described herein, the iloprost is present in a concentration between about 0.1%—about 5% by weight. In additional embodiments of the glass formulations described herein, the iloprost is present in a concentration between about 0.1%-about 1% by weight. In some embodiments of the present invention, the glass formulations comprise one or more hydrophobic derivatized carbohydrate (HDC) or modified glycoside. The hydrophobic derivatized carbohydrate or modified glycoside may be an oligosaccharizde ester derivative. For example, in some embodiments, the glass formulations comprise TR153. TR153 is 6:6′-bis(β-Tetraacetyl glucuronyl)hexaacetyl trehalose. (See R. Alcock et al.; Modifying the release of leuprolide from spary dried OED microparticles, Journal of Controlled Release 82: 429-440 (2002) and I.G. Davidson et al. Release mechanism of insulin encapsulated in trehalose ester derivative microparticles delivered via inhalation, Journal of Pharmaceuticals 254:211-222 (2003), the disclosures of which are incorporated herein by reference in their entireties.) The structure of TR153 is depicted below:

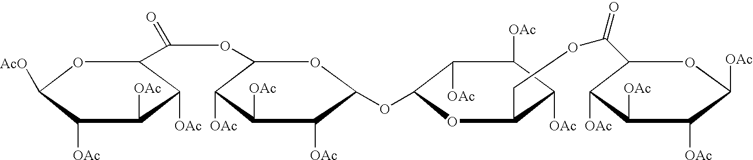

In some embodiments, the one or more HDC or modified glycoside are present in a concentration between about 99.9%-about 10% by weight. In other embodiments of the glass formulations described herein, the one or more stabilizing polyols are present in a concentration between about 99.7%-about 50% by weight. In some embodiments of the glass formulations described herein, the one or more stabilizing polyols are present in a concentration between about 99.7%-about 65% by weight.

In some embodiments of the glass formulations described herein, the glass formulation comprises one or more surfactants. In some embodiments, the one or more surfactants are dipalmitoyl phosphatidylglycerol or dipalmitoyl phosphatidylcholine. In some embodiments, the one or more surfactants are present at a concentration between about 0.01%-about 30% by weight. In other embodiments of the glass formulations described herein, the one or more surfactants are present in a concentration between about 0.1%-about 20% by weight. In further embodiments of the glass formulations described herein, the one or more surfactants are present in a concentration between about 0.1%-about 10% by weight. In additional embodiments of the glass formulations described herein, the one or more surfactants are present in a concentration between about 0.1%-about 5% by weight.

In some embodiments of the present invention, the glass formulations comprise one or more stabilizing polyols. For example, in some embodiments, the glass formulations comprise trehalose. In some embodiments of the glass formulations described herein, the one or more stabilizing polyols are present in a concentration between about 0%-about 50% by weight. In other embodiments of the glass formulations described herein, the one or more stabilizing polyols are present in a concentration between about 0.1%-about 30% by weight. In some embodiments of the glass formulations described herein, the one or more stabilizing polyols are present in a concentration between about 0.1%-about 20% by weight.

In some embodiments, the glass formulation comprises between about 0.1%-about 5% iloprost by weight, between about 0.1%-about 5% dipalmitoyl phosphoglycerol and/or between about 0.1%-5% dipalmitoyl phosphatidylcholine by weight and between about 0%-about 20% trehalose by weight with the remainder of the formulation comprising TR153. In some embodiments, the glass formulation comprises less than 5% dipalmitoyl phosphoglycerol by weight. In some embodiments, the glass formulations are solubilized in a solvent comprising less than 20% water. In some embodiments, the particles in the glass formulation have dimensions of less than about 10 microns in diameter. In some embodiments, the particles in the glass formulation have a mass median aerodynamic diameter of about 0.2 microns-about 10 microns. In some embodiments, the particles in the glass formulation have a mass median aerodynamic diameter of about 0.5 microns-about 5 microns. In some embodiments, the particles in the glass formulation have a mass median aerodynamic diameter of about 1 micron in diameter. In some embodiments, the particles in the glass formulation have a median size of about 2.2 microns in diameter. In some embodiments, the particles in the glass formulation have a mass median aerodynamic diameter of about 5 microns in diameter. In some embodiments, the particles in the glass formulation have a mass median aerodynamic diameter of about 8 microns in diameter. In some embodiments, the glass formulation does not contain dipalmitoyl phosphatidylglycerol but does contain dipalmitoyl phosphatidylcholine. In some embodiments, the iloprost is present at a concentration of about 0.3% by weight.

In some embodiments, the glass formulation provides a bioavailability of more than 10% over a 24 hour period following a pulmonary dose when assessed in the dog model described in Example 28 below. In some embodiments the glass formulation maintains plasma iloprost levels within a 10-fold range for more than about 2 hours and less than about 24 hours when assessed in the dog model described in Example 28 below.

In some embodiments the glass formulation comprises no more than 1.5% total iloprost related substances or decomposition products when stored at ambient temperatures (20-25 degrees Celsius) for two years.

In an embodiment of the present invention, a combination therapy is disclosed for treating pulmonary hypertension. In one aspect of this embodiment, a pharmaceutical agent other than iloprost is administered in addition to the microparticles comprising iloprost. The microparticles may be any of the microparticles discussed herein. The other pharmaceutical agent may be contained in the same microparticle as the iloprost or it may be in a separate microparticle. Alternatively, the other pharmaceutical agent may be administered in a form other than a microparticle. The other pharmaceutical agent may be administered at the same time as the microparticles comprising iloprost or may be administered at any desired time before and/or after administration of the microparticles comprising iloprost.

In one embodiment, the pharmaceutical agent other than iloprost may be an endothelin receptor antagonist, that modulates the vasostate (e.g., vasodilation) of blood vessels through a mechanism which is distinct from that of iloprost. Preferably, the endothelin receptor antagonist is selected from the group consisting of bosentan (Tracleer™, Actelion), ambrisentan (Myogen) and sitaxentan (Encysive Pharmaceuticals).

In another embodiment, the pharmaceutical agent to be administered in addition to iloprost is a pharmaceutical agent which modulates prostacyclin activity, bioavailability, half-life, or ameliorates an undesirable side-effect of the prostacyclin. In one preferred embodiment, the pharmaceutical agent to be administered in addition to iloprost is a PDE inhibitor adapted to enhance the prostacyclin activity, preferably selected from the group consisting of enoximone, milrinone (Primacor®), Amrinone (Inocor®), sildenafil (Viagra®), tadalafil (Cialis®) and vardenafil (LEVITRA®).

Epoprostenol Derivatives

In some embodiments, the pharmaceutical agent to be administered in addition to iloprost is an epoprostenol derivative. A continuous infusion of prostacyclin (Flolan®, GlaxoSmithKline) was the first therapy shown to reduce mortality in a controlled study of patients with severe pulmonary hypertension. However, its use is associated with a number of serious drawbacks (Barst R. J. et al. 1996 N Engl J Med 334:296-301; Badesch D. B. et al. 2000 Ann Intern Med 132:425-434). The lack of pulmonary selectivity results in systemic side effects, tolerance leads to progressive increases in the dose, and there may be recurrent infections of the intravenous catheter. As an alternative, inhaled nitric oxide possesses pulmonary selectivity, but it is less potent than prostacyclin in the pulmonary vasculature. Moreover, an interruption in the inhalation of continuous nitric oxide may cause rebound pulmonary hypertension. Designed to combine the beneficial effects of prostacyclin with those of an inhalational application, aerosolized prostacyclin was found to be a potent pulmonary vasodilator in patients with acute respiratory failure, exerting preferential vasodilatation in well-ventilated lung regions (Walmrath D. et al. 1993 Lancet 342:961-962; Walmrath D. et al. 1995 Am J Respir Crit Care Med 151:724-730; Walmrath D. et al. 1996 Am J Respir Crit Care Med 153:991-996; Zwissler B. et al. 1996 Am J Respir Crit Care Med 154:1671-1677). Similar results were obtained in spontaneously breathing patients who had lung fibrosis and severe pulmonary hypertension (Olschewski H. et al. 1999 Am J Respir Crit Care Med 160:600-607).

Three epoprostenol analogs have been studied in the treatment of PAH: treprostinil (Remodulin®, United Therapeutics), beraprost, and iloprost. Treprostinol is a stable analogue of epoprostenol, which is given continuously subcutaneously. Escalation of dosage has been limited by significant infusion site pain. Thus many patients do not receive therapeutic doses. Beraprost is active orally and has shown a benefit in a study in PAH at 3 and 6 months but not at 9 or 12 months (Barst, R J, J Am Coll Cardiol, 2003. Jun. 18;41(12):2119-25. As discussed above, the iloprost is administered in microparticle form. In some embodiments, the iloprost is administered at a frequency which is less than that which would be required if the iloprost were not administered in microparticle form. Dosing frequency may be further reduced by administering an agent in addition to iloprost which has a therapeutic effect on the pulmonary hypertension through a different mechanism and which may act synergistically with iloprost.

Endothelin Receptor Antagonists (ETRA)

In some embodiments, the pharmaceutical agent which is administered in addition to iloprost is an endothelin receptor antagonist. There is increasing evidence that endothelin-1 has a pathogenic role in pulmonary arterial hypertension and that blockade of endothelin receptors may be beneficial. Endothelin-1 is a potent endogenous vasoconstrictor and smooth-muscle mitogen that is overexpressed in the plasma and lung tissue of patients with pulmonary arterial hypertension. There are two classes of endothelin receptors: Endothelin A, ET-A and Endothelin B, ET-B receptors, which play significantly different roles in regulating blood vessel diameter. The binding of endothelin to ET-A receptors located on smooth muscle cells causes vasoconstriction, whereas the binding of endothelin to ET-B receptors located on the vascular endothelium causes vasodilatation through the production of nitric oxide. This latter activity of the ET-B receptor is thought to be counter-regulatory and protects against excessive vasoconstriction.

Therefore, another attractive approach to treating pulmonary hypertension has been the blockade of these endothelin receptors. Two types of ETRAs have been developed: dual ETRAs, which block the receptors for both ET-A and ET-B, and selective ETRAs, which block only the ET-A receptor.

Dual Endothelin Receptor Antagonist

The first generation ETRAs are non-selective and block both the ET-A and ET-B receptors. Bosentan (Tracleer™) is the first FDA approved ETRA (see U.S. Pat. No. 5,292,740; incorporated herein in its entirety by reference thereto). Two placebo controlled trials of bosentan (an endothelin receptor A and B antagonist) have been conducted (Channick R. N. et al. 2001 Lancet 358:1119-1123; Rubin L. J. et al. 2002 N Engl J Med 346:896-903). The six minute walk test improved in the whole group, but the improvement was greater when the drug was used in higher doses. However, liver toxicity occurred with the higher dose.

Selective Endothelin Receptor Antagonist

Second generation ETRAs bind to the ET-A receptor in preference to the ET-B receptor. Currently, there are two selective ETRAs in clinical trials: sitaxsentan and ambrisentan (BSF 208075). A pure endothelin A antagonist, sitaxsentan has been used in an open pilot study. This showed an improvement in the six minute walk test and a decrease in pulmonary vascular resistance of 30% (Barst R. J. et al. 2000 Circulation 102:II-427).

A more potent endothelin compound, TBC3711 (Encysive Pharmaceuticals), entered Phase I testing in December 2001. This drug holds potential for treating chronic heart failure and essential hypertension.

There are small clinical trials of using bosentan in patients that are already on other medications for the treatment of pulmonary hypertension (Hoeper M. M. et al. 2003 in: “Pulmonary Hypertension: Clinical”, Abstr. A275, May 18, 2003; Pulmonary Hypertension Roundtable 2002, Phassociation.org/medical/advances in PH/spring 2002). In a preferred embodiment of the present invention, the combination therapy comprises iloprost and bosentan acting in combination through distinct mechanisms of action, preferably synergistically, to treat pulmonary hypertension. In yet another preferred embodiment, iloprost is combined with sitaxentan. In yet another embodiment, iloprost is combined with ambrisentan. In yet another embodiment iloprost is aerosolized and administered in combination with bosentan, or sitaxentan, or ambrisentan. In another embodiment, iloprost is combined with TBC3711 in combination therapy of pulmonary hypertension.

Nitric Oxide Production

In some embodiments, the pharmaceutical agent which is administered in addition to iloprost is nitric oxide or a pharmaceutical agent which is a substrate for nitric oxide. Endothelial production of nitric oxide is diminished with pulmonary hypertension, prompting attempts to reverse this defect either by giving continuous inhaled nitric oxide, which is effective but difficult to administer, or by increasing the substrate for nitric oxide L-arginine (Nagaya N. et al. 2001 Am J Respir Crit Care Med 163:887-891). A trial of supplementation with L-arginine is currently under way.

PDE Inhibitors

In some embodiments, the pharmaceutical agent which is administered in addition to iloprost is a PDE inhibitor. In addition to increasing the supply of nitric oxide, attempts to directly increase cyclic nucleotide second messenger levels in the smooth muscle cells have been made. Sildenafil used for erectile dysfunction blocks the enzyme phosphodiesterase type 5 present in the corpus cavernosum of the penis and also the lungs. This raises the possibility that a phosphodiesterase inhibitor, preferably a PDE type 5 inhibitor such as sildenafil, could be a relatively selective pulmonary vasodilator. There is empirical evidence supporting the inventor's selection of PDE inhibitors as a target compound in a combination therapy (see e.g., Michelakis E. et al. 2002 Circulation 105:2398-2403; Ghofrani H. et al. 2002 Lancet 360:895-900; the disclosures of which are incorporated herein in their entirety by reference).

Although aerosolized prostacyclin (PGI₂) has been suggested for selective pulmonary vasodilation as discussed above, its effect rapidly levels off after termination of nebulization. Stabilization of the second-messenger cAMP by phosphodiesterase (PDE) inhibition has been suggested as a strategy for amplification of the vasodilative response to nebulized PGI₂. Lung PDE3/4 inhibition, achieved by intravascular or transbronchial administration of subthreshold doses of specific PDE inhibitors, synergistically amplified the pulmonary vasodilatory response to inhaled PGI₂, concomitant with an improvement in ventilation-perfusion matching and a reduction in lung edema formation. The combination of nebulized PGI₂ and PDE3/4 inhibition may thus offer a new concept for selective pulmonary vasodilation, with maintenance of gas exchange in respiratory failure and pulmonary hypertension (Schermuly R. T. et al. 2000 J Pharmacol Exp Ther 292:512-20). There are some reports of small clinical studies showing that such combination therapy may be efficacious in the treatment of pulmonary hypertension (Ghofrani et al. 2002 Crit Care Med 30:2489-92; Ghofrani et al. 2003 J Am Coll Cardiol 42:158-164; Ghofrani et al. 2002 Ann Intern Med 136:515-22).

Isozymes of cyclic-3′,5′-nucleotide phosphodiesterase (PDE) are a critically important component of the cyclic-3′,5′-adenosine monophosphate (cAMP) protein kinase A (PKA) signaling pathway. The superfamily of PDE isozymes consists of at least nine gene families (types): PDE1 to PDE9. Some PDE families are very diverse and consist of several subtypes and numerous PDE isoform-splice variants. PDE isozymes differ in molecular structure, catalytic properties, intracellular regulation and location, and sensitivity to selective inhibitors, as well as differential expression in various cell types.

A phosphodiesterase (PDE) inhibitor is defined herein as any drug used in the treatment of pulmonary hypertension that works by blocking the inactivation of cyclic AMP. There are five major subtypes of phosphodiesterase (PDE); the drugs enoximone (inhibits PDE IV) and milrinone (Primacor®) (inhibits PDE IIIc) are most commonly used medically. Other phosphodiesterase inhibitors include Amrinone (Inocors) used to improve myocardial function, pulmonary and systemic vasodilation, and sildenafil (Viagra®), tadalafil (Cialis®) and vardenafil (LEVITRA®)—selective phosphodiesterase V inhibitors.

http://www.businesswire.com/webbox/bw.042803/231185439.htm reported clinical data on tadalafil, showing that 79 percent of U.S. men of diverse ethnic origin with erectile dysfunction (ED) participating in a clinical trial reported improved erections after treatment with the investigational drug, compared to 19 percent of those receiving placebo. The results of this new study conducted in the U.S. and Puerto Rico were presented today at the 98th Annual Meeting of the American Urological Association in Chicago. ED is a condition that affects an estimated 152 million men worldwide.

Tadalafil (Cialis®) is a PDE5 inhibitor developed by Lilly ICOS LLC for the treatment of erectile dysfunction. Tadalafil is available by prescription in Europe, Australia, New Zealand, and Singapore. A U.S. regulatory decision for tadalafil is anticipated to occur in the second half of 2003.

“Treatment with Cialis significantly improved erectile function, including increasing the number of successful attempts at penetration and intercourse, and the improvement of erections,” said Allen Seftel, M. D, study author and associate professor of urology at the University Hospitals of Cleveland. “I was pleased with the tolerability profile seen in these U.S. men of diverse ethnic origin, with mild to severe ED.”

In a randomized, placebo-controlled clinical study designed to evaluate the efficacy and safety of Cialis in men with mild-to-severe ED, 207 participants in the U.S. and Puerto Rico were assigned to receive either a 20 mg dose of Cialis or placebo over a 12-week period. The treatment phase was preceded by a treatment-free period of four weeks to determine baseline erectile function. Patients were advised to take the drug as needed, at the time of their choosing prior to sexual activity, and were informed that Cialis may be effective for up to 36 hours. In the study, men were advised to eat normal meals with no restrictions on fat content.

In the study, 79 percent of patients treated with Cialis reported improved erections, as determined by the Global Assessment Question, compared to 19 percent on placebo. Additional findings revealed that 77 percent of attempts at vaginal penetration, as recorded in the Sexual Encounter Profile diary, were successful in men taking Cialis, compared to 43 percent on placebo (p less than 0.001). Furthermore, men taking Cialis were able to complete 64 percent of attempts for sexual intercourse versus 23 percent of attempts for men taking placebo (p less than 0.001). Finally, men taking Cialis achieved statistically significant improvements compared to placebo for all other endpoints.

The most commonly reported (greater than or equal to 5 percent) treatment-emergent adverse effects in the study were headache, back pain, and upset stomach. The number of patients taking Cialis who discontinued the study because of adverse events was 5 percent, compared to 2 percent for placebo.

A second clinical study presented at the annual meeting of the American Urological Association was designed to evaluate the long-term safety and tolerability of Cialis in 1,173 men with ED, who had previously been enrolled in Phase III clinical studies of Cialis conducted in multiple countries worldwide. These men included those who had a range of co-morbid conditions associated with ED, such as cardiovascular disease and diabetes mellitus. Data reported were from patients who had completed at least one year of treatment.

All study participants initially received 10 mg of Cialis; during the assessment period, 83 percent (n=970) of these patients increased their dosage to 20 mg. Patients were advised to take the treatment as needed prior to sexual activity.

Similar to other Cialis clinical trials, the most commonly reported treatment-emergent adverse effects in the study were headache and upset stomach. Five percent of patients discontinued the study due to side effects. The discontinuation rate in this study for any individual adverse event was less than 1%.

Bayer reported that LEVITRA® (vardenafil HC1) has been approved by the U.S. Food and Drug Administration (FDA) for the treatment of erectile dysfunction (ED). http://www.pharma.bayer.com/servlet/Satellite?pagename=Bayer/BPP/Article. Levitra is expected to be available in pharmacies nationwide within the next few weeks.

“In clinical trials, Levitra was shown to work quickly. More importantly, Levitra was shown to improve the sexual response for the majority of men the first time they took it, and it worked consistently over time,” said Myron Murdock, M.D., Levitra investigator and nationally recognized expert in the field of male sexual dysfunction.

Bayer and GSK evaluated Levitra in an extensive clinical trial program that included more than 50 trials involving more than 5,700 men. Results from phase III clinical studies showed that Levitra:
Helped men get and keep an erection sufficient for satisfactory sexual performance
Provided first-time success and reliable improvement of erection quality for many men
Worked in men of various ages and race and in those with co-existing medical conditions, such as diabetes, and in men who have had their prostate removed
Demonstrated a rapid response, allowing a man to initiate or respond to sexual stimulation when the time is right
Can be taken without regard to meals making it convenient for use

Levitra is a medicine that may be used up to once a day to treat erectile dysfunction (ED). Levitra is for use by prescription only. Men taking nitrate drugs, often used to control chest pain (also known as angina), should not take Levitra. Men who use alpha blockers, sometimes prescribed for high blood pressure or prostate symptoms, also should not take Levitra. Such combinations could cause blood pressure to drop to an unsafe level. The most commonly reported side effects are headache, flushing, and stuffy or runny nose. Men who experience an erection for more than four hours should seek immediate medical attention.

For detailed information about Levitra, see www.Levitra.com, the disclosure of which is incorporated herein in its entirety by reference.

Calcium Channel Blockers

In some embodiments, the pharmaceutical agent which is administered in addition to iloprost is a calcium channel blocker. Calcium channel blockers, or antagonists, act by blocking the entry of calcium into muscle cells of heart and arteries so that the contraction of the heart decreases and the arteries dilate. With the dilation of the arteries, arterial pressure is reduced so that it is easier for the heart to pump blood. This also reduces the heart's oxygen requirement. Calcium channel blockers are useful for treating PPH. Due to blood pressure lowering effects, calcium channel blockers are also useful to treat high blood pressure. Because they slow the heart rate, calcium channel blockers may be used to treat rapid heart rhythms such as atrial fibrillation. Calcium channel blockers are also administered to patients after a heart attack and may be helpful in treatment of arteriosclerosis.

Calcium channel blockers which are within the scope of this invention include, but are not limited to: amlodipine (U.S. Pat. No. 4,572,909); bepridil (U.S. Pat. No. 3,962,238); clentiazem (U.S. Pat. No. 4,567,175); diltiazem (U.S. Pat. No. 3,562,257); fendiline (U.S. Pat. No. 3,262,977); gallopamil (U.S. Pat. No. 3,261,859); mibefradil (U.S. Pat. No. 4,808,605); prenylamine (U.S. Pat. No. 3,152,173); semotiadil (U.S. Pat. No. 4,786,635); terodiline (U.S. Pat. No. 3,371,014); verapamil (U.S. Pat. No. 3,261,859); aranidipine (U.S. Pat. No. 4,446,325); bamidipine (U.S. Pat. No. 4,220,649): benidipine (European Patent Application Publication No. 106,275); cilnidipine (U.S. Pat. No. 4,672,068); efonidipine (U.S. Pat. No. 4,885,284); elgodipine (U.S. Pat. No. 4,952,592); felodipine (U.S. Pat. No. 4,264,611); isradipine (U.S. Pat. No. 4,466,972); lacidipine (U.S. Pat. No. 4,801,599); lercanidipine (U.S. Pat. No. 4,705,797); manidipine (U.S. Pat. No. 4,892,875); nicardipine (U.S. Pat. No. 3,985,758); nifedipine (U.S. Pat. No. 3,485,847); nilvadipine (U.S. Pat. No. 4,338,322); nimodipine (U.S. Pat. No. 3,799,934); nisoldipine (U.S. Pat. No. 4,154,839); nitrendipine (U.S. Pat. No. 3,799,934); cinnarizine (U.S. Pat. No. 2,882,271); flunarizine (U.S. Pat. No. 3,773,939); lidoflazine (U.S. Pat. No. 3,267,104); lomerizine (U.S. Pat. No. 4,663,325); bencyclane (Hungarian Patent No. 151,865); etafenone (German Patent No. 1,265,758); and perhexiline (British Patent No. 1,025,578). The disclosures of all such patents and patent applications are incorporated herein by reference.

Preferred calcium channel blockers comprise amlodipine, diltiazem, isradipine, nicardipine, nifedipine, nimodipine, nisoldipine, nitrendipine, and verapamil, or, e.g., dependent on the specific calcium channel blockers, a pharmaceutically acceptable salt thereof.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having at least one acid group (for example COOH) can also form salts with bases. Corresponding internal salts may furthermore be formed, if a compound of formula comprises e.g., both a carboxy and an amino group.

In accordance with one embodiment a second generation calcium antagonist, such as amlodipine, is the pharmaceutical agent which is administered in addition to iloprost. In some embodiments, both the iloprost and the calcium antagonist are administered in a sustained release dosage form. Preferably, the dosages of iloprost and the calcium antagonist and their release form are optimized for the treatment of hypertensive patients.

The following examples are meant to illustrate but not limit the invention.

### EXAMPLES

In the examples below, where porosity of microparticles is determined, the following procedure may be used: TAP Density (Transaxial Pressure Density as a measure of tap density) for the microparticles is determined using a Micromeritics GeoPyc Model 1360 or other suitable device. Envelope density for the microparticles is estimated from the TAP density (EQ. 5). Absolute density is determined via helium pycnometry using a Micromeritics AccuPyc Model 1330 or another suitable device. The absolute densities of the polymer, pharmaceutical agent, and phospholipid is determined, and a weighted average value is used for the absolute density of the microparticles. The porosity is calculated based on EQ.6 above. Where percent porosity is to be determined, the value of porosity (based on EQ.6) is multiplied by 100%.

In some embodiments, the in vitro release rate of the iloprost or another pharmaceutical agent to be administered in addition to iloprost may be determined using the following procedure. Microparticles comprising the iloprost and/or another pharmaceutical agent to be administered in addition to iloprost are suspended in PBS-SDS (Phosphate Buffered Saline—0.05% Sodium Dodecyl Sulfate) such that the nominal concentration of the iloprost and/or another pharmaceutical agent to be administered in addition to iloprost in the suspension is 1 mg/mL. A sample of the suspension is then added to a large volume of PBS-SDS at 37 degrees C., such that the theoretical pharmaceutical agent concentration at 100% release is 0.75 micrograms/mL. The resulting diluted suspension is maintained at 37 degrees C. in an incubator on a rocker. To determine the release rate of pharmaceutical agent from the microparticles, samples of the release media are taken over time, the microparticles are separated from the solution, and the solution pharmaceutical agent concentration is monitored via HPLC with detection at a wavelength appropriate for detection of iloprost and/or a pharmaceutical agent to be administered in addition to iloprost. The HPLC column may be any column suitable for separating iloprost and/or another pharmaceutical agent to be administered in addition to iloprost from other components of the release media. The mobile phase may be any phase suitable for separation of the iloprost and/or another pharmaceutical agent to be administered in addition to iloprost from the other components of the release media.

In the examples below, where geometric particle size is described, the volume average size may be measured using a Coulter Multisizer H with a 50 micrometer aperture or other suitable device.

If desired, powders may be dispersed in an aqueous vehicle containing Pluronic F127 and mannitol using vortexing and sonication. The resulting suspensions are then diluted into electrolyte for analysis.

### Example 1

### In Vitro Analysis of Effect of Microparticle Porosity on Release of Iloprost or Another Pharmaceutical Agent to be Administered in Addition to Iloprost

Microspheres containing iloprost and/or another pharmaceutical agent to be administered in addition to iloprost are prepared, using materials obtained as follows: iloprost is obtained from Schering AG or another suitable supplier; phospholipid (DPPQ is obtained from Avanti Polar Lipids Inc. (Alabaster, Ala.) or another suitable supplier; polymer (PLGA) is obtained from BI Chemicals (Petersburg, Va.) or another suitable supplier; ammonium bicarbonate is obtained from Spectrum Chemicals (Gardena, Calif.); and methylene chloride is obtained from EM Science (Gibbstown, N.J.) or another suitable supplier.

Microparticles having differing levels of porosity and comprising iloprost and/or another pharmaceutical agent to be administered in addition to iloprost are prepared using different combinations of any of the particle components discussed above so as to generate microparticles having differing levels of porosity but containing the same amount of iloprost and/or another pharmaceutical agent to be administered in addition to iloprost. In one example, the microparticles comprising iloprost and/or another pharmaceutical agent to be administered in addition to iloprost and having different levels of porosity are prepared as follows. For a given amount of iloprost and/or another pharmaceutical agent to be administered six different microparticles of varying porosity may be formulated as follows. Microparticles 1-5 are prepared as follows. For each microsphere lot (Particles 1-6) 8.0 g of PLGA, 0.72 g of DPPC, and a desired amount of iloprost and/or another pharmaceutical to be administered in addition to iloprost are dissolved into 364 mL of methylene chloride at 20 degrees C. For reference, Particle 1 is prepared without a pore forming agent, and the process conditions and solids content of the solution to the spray dryer is used to create the porosity of the microspheres. Particles 2-6 are prepared using the pore forming agent, ammonium bicarbonate to create microspheres having porosities greater than Particle 1. For example, for Particles 2-6, a stock solution of the pore forming agent is prepared by dissolving 4.0 g of ammonium bicarbonate into 36 mL of RO/DI water at 20 degrees C. For each lot, a different ratio of the ammonium bicarbonate stock solution is combined with the iloprost and/or other pharmaceutical agent/polymer solution described above and emulsified using a rotor-stator homogenizer. The resulting emulsion is spray dried on a benchtop spray dryer using an air-atomizing nozzle and nitrogen as the drying gas. Spray drying conditions are as follows: 20 mL/min emulsion flow rate, 60 kg/hr drying gas rate and 21 degrees C. outlet temperature. The product collection container is detached from the spray dryer and attached to a vacuum pump, where it is dried for at least 18 hours. For example, the following ratios of volume pore forming agent: pharmaceutical agent/polymer solution may be used: Particle 2: 1:49, Particle 3: 1:24, Particle 4: 1:10, Particle 5: 1:49, Particle 6: 1:19). Alternatively, other ratios of volume pore forming agent: pharmaceutical agent/polymer solution may be used to generate particles having other desired levels of porosity. In addition, it will be appreciated that other pore forming agents and stock solutions compatible with iloprost and/or another pharmaceutical agent to be administered in addition to iloprost may be used.

The release rate of iloprost and/or another pharmaceutical agent to be administered in addition to iloprost may be measured in vitro to identify those formulations having a desired release rate in a given amount of time. Thus, the level of porosity can be used to adjust the amount of pharmaceutical agent released after a certain period of time, and particles having a desired release profile can be further analyzed in vivo.

### Example 2

### Production of Radiolabeled Microparticles Containing Iloprost or Another Pharmaceutical Agent to be Administered in Addition to Iloprost for Use in In Vivo Analysis

Microparticles containing iloprost and/or another pharmaceutical agent to be administered in addition to iloprost are produced as described above in Example 1.

The dried microspheres are then radiolabeled with technetium or another suitable isotope. Alternatively, other suitable detectable labels may be used. The labeled microparticles are transferred to a stainless steel mixing vessel and manually mixed with lactose. The mixed materials are then blended on a Turbula shaker-mixer, and the blended material is manually filled into gelatin capsules, such as size 3 Coni-Snap capsules available from Capsugel, Greenwood, S.C. or other suitable capsules.

### Example 3

### Administration of Labeled Microparticles To Human Subjects by Inhalation

A randomized, open-label, single-dose, single-centre, crossover study or other desired in vivo analysis in healthy volunteers (IO subjects) is conducted comparing pharmacokinetics and pulmonary deposition of the labeled microparticles containing iloprost and/or another pharmaceutical agent to be administered in addition to iloprost produced as described above delivered by dry powder inhaler and an immediate release iloprost formulation or formulation of another pharmaceutical agent to be administered in addition to iloprost (or other desired reference formulation) which are delivered using a commercial dry powder inhaler using a desired number of actuations to provide a desired dosage. For example, if desired, the radiolabeled microparticles prepared as described in Example 2 may be used. If desired, the doses administered for both the microparticle formulation and the reference formulation may be significantly higher than would be administered under therapeutic conditions, to ensure plasma levels of iloprost and/or another pharmaceutical agent to be administered in addition to iloprost which is above the level of detection and thus allow the in vivo release profile of the microspheres to be assessed. Plasma concentrations of iloprost and/or another pharmaceutical agent to be administered in addition to iloprost are measured at 0, 2, 4, 6, 8, 12, 20, 30, 45, 60 minutes, and 1.5, 2, 3, 4, 6, 8, 10 and 12 hours after the final inhalation of each dosing period or at other desired time points. Plasma samples are analyzed using a validated LC/MS/MS method. The plasma profiles adjusted for actual inhaled dose are determined.

Non-compartmental analysis is performed on the plasma curves. The results indicate a significant difference in the mean absorption time following inhalation for the microparticles containing iloprost and/or another pharmaceutical agent to be administered in addition to iloprost (MATi,h) versus the reference formulation. This clearly indicates that the iloprost and/or the pharmaceutical agent to be administered in addition to iloprost is absorbed slowly into the systemic circulation after inhalation of the microparticles as compared to inhalation of the immediate release or other reference formulation.

If desired, the regional distribution of the microparticles in the lung may be determined via gamma scintigraphy.

### Example 4

### Preparation of PLGA:DAPC Drug Delivery Particles

30 grams of PLGA (50:50) (IV 0.4 dL/g Boehringer Ingelheim or another suitable supplier), 1.8 g of diarachidoylphosphatidylcholine (Avanti, Birmingham, Ala.) and 495 mg of Azure A (Sigma Chemicals, St. Louis, Mo. or another suitable supplier) are dissolved in 1000 ml of methylene chloride. The solution is pumped at a flowrate of 20 mL/min and spray dried using a Bucchi Lab spray dryer or other suitable device. The inlet air temperature is 40° C. The dried microparticle powder is collected and stored at −20° C. until analysis. Size of the microparticles is performed using a Coulter multisizer II or other suitable L device. The microparticles have a volume average mean diameter of 5.982 microns.

18 grams of PLGA (50:50) (IV 0.4 dL/g Boehringer Ingelheim or another suitable supplier) and 1.08 g of diarachidoylphosphatidylcholine (Avanti, Birmingham, Ala. or another suitable supplier) are dissolved in 600 mL of methylene chloride. 38.9 mg of Eosin Y (Sigma Chemicals or another suitable supplier) is dissolved in 38.9 mL of a 0.18 g/ml ammonium bicarbonate solution. The eosin solution is emulsified with the polymer solution using a Silverson homogenizer at 7000 rpm for 8 minutes. The solution is pumped at a flowrate of 20 mL/min and spray dried using a Bucchi Lab spray dryer or other suitable device. The inlet air temperature is 40° C. The dried microparticle powder is collected and stored at −20° C. until analysis. Size analysis of the microparticles is performed using a Coulter multisizer II. The microparticles have a volume average mean diameter of 6.119 microns.

Some of the methods and materials employed in Examples 5 and 6 are described in U.S. application Ser. No. 09/211,940, filed Dec. 15, 1998, in U.S. application Ser. No. 08/739,308, filed Oct. 29, 1996, now U.S. Pat. No. 5,874,064, in U.S. application Ser. No. 08/655,570, filed May 24, 1996, in U.S. application Ser. No. 09/194,068, filed May 23, 1997, in PCT/US97/08895 application filed May 23, 1997, in U.S. application Ser. No. 08/971,791, filed Nov. 17, 1997, in U.S. application Ser. No. 08/784,421, filed Jan. 16, 1997, now U.S. Pat. No. 5,855,913 and in U.S. application Ser. No. 09/337,245, filed on Jun. 22, 1999, all of which are incorporated herein by reference in their entirety.

Materials

Leucine is obtained from Spectrum Chemical Company. DPPC is obtained from Avanti Polar Lipids (Alabaster, Ala.) or another suitable supplier.

Spray Drying

A Mobile Minor spray-drier from Niro or other suitable spray drier is used. The gas employed is dehumidified air. The gas temperature may range from about 80 to about 150 degrees C. or may be any other suitable temperature. The atomizer speed may range from about 15,000 to about 50,000 RPM or may be any other suitable speed. The gas rate may be 70 to 92 kg/hour or any other suitable gas rate and the liquid feed rate may range from about 50 to about 100 ml/minute or may be any other suitable feed rate.

Geometric Size Distribution Analysis

Size distributions are determined using a Coulter Multisizer II or other suitable device. Approximately 5-10 mg of powder is added to 50 mL isoton II solution until the coincidence of particles is between 5 and 8%. Greater than 500,000 particles are counted for each batch of spheres.

Aerodynamic Size Distribution Analysis

Aerodynamic size distribution is determined using an Aerosizer/Aerodispenser (Amherst Process Instruments, Amherst, Mass. or other suitable device). Approximately 2 mg powder is introduced into the Aerodisperser and the Aerodynamic size is determined by time of flight measurements.

### Example 5

A mixture including 40 weight % of an amino acid and 60 weight % DPPC is formed in a 70/30 vol/vol ethanol-water co-solvent and spray-dried and the geometric aerodynamic diameters for the particles are determined. In addition, the hydrophobicity and tap density may also be determined. Tap density may be determined using the equation provided above. For example, the amino acid may be leucine, isoleucine, phenylalanine, glutamine, or glutamate. The characteristics of the particles may be as described in Table 3.

TABLE 3

| Amino acid | hydrophobicity | MMGD | MMAD | Est. tap density |
|---|---|---|---|---|
| Leucine | 0.943 | 7.9 | 3.0 | 0.11 |
| Isoleucine | 0.943 | 8.1 | 2.7 | 0.14 |
| Phenylalanine | 0.501 | 7.9 | 3.8 | 0.23 |
| Glutamine | 0.251 | 6.5 | 4.4 | 0.45 |
| Glutamate | 0.043 | 5.1 | 4.1 | 0.64 |

Microparticles having desired characteristics may be selected for further analysis according to Examples 1-3.

### Example 6

Microparticles containing iloprost and/or another pharmaceutical agent to be administered in addition to iloprost and a desired amino acid or group of amino acids are prepared as described above. The mass median geometric diameter and the mass median aerodynamic diameter is determined. In vitro and in vivo analyses are performed as described in Examples 1-3.

### Example 7

### Methods of Making Powder SP Vitreous Solid Dose Delivery Systems

a) Incorporation of Active in SP Vitreous Delivery Vehicle to Yield Micronized Powders

Glasses are formed by drying 20% solutions of either trehalose, lactitol, palatinit, GPM or GPS, containing a MWPB and iloprost and/or another pharmaceutical agent to be administered in addition to iloprost by freeze-drying under vacuum (80 mTorr) for 16 hrs. The glasses are powdered using a Trost air-jet mill. Particle size in the micronized powders is measured using a Malvern Mastersizer laser particle sizer. The results obtained with micronized powders obtained from an original solution of 0.5 M trehalose and 0.5 M calcium lactate show a monodisperse particle distribution with mean particle diameters of 1.1 microns. The powders containing MWPB remain a free-flowing powder and show no change in particle size or clumping and uptake of water on extended exposure to ambient temperatures and humidities.

b) Incorporation of Active in SP Vitreous Delivery Vehicle to Yield Spray-Dried Powders

20% solutions of trehalose containing MWPB salts and iloprost and/or another pharmaceutical agent to be administered in addition to iloprost are dried in a Buchi or Lab-Plant spray drier at a pump speed of 500-550 ml/hr and an inlet temperature of 180° C. Particle size is measured using a SympaTec laser particle sizer. The spray-dried powders show a monodisperse particle distribution with a sufficiently narrow peak size distribution for effective use as particles in a powder ballistic device. Particle size analysis of a spray-dried powder produced by spray drying a mixture of 0.5 M trehalose and 0.5 M calcium lactate on a Lab-Plant spray drier shows a mean particle diameter of 8.55 microns and illustrates the tight peak distribution obtained. Variation of the mean particle size can be achieved by varying either the composition of the mixture to be spray dried or the characteristics of the spray drier nozzle assembly used. The peak distribution shows a narrow range with a mean particle size of 7.55 microns.

Particles obtained by different spray-drying processes are equally suitable to provide compositions for ballistic delivery. The ability to vary particle size results in compositions with different penetrative characteristics.

c) Incorporation of Active in SP Vitreous Delivery Vehicle by Drying from Organic Solvents

A solution of iloprost and/or another pharmaceutical agent to be administered in addition to iloprost in a 1.1 mixture of ethanol:water, containing 20% trehalose, is air-dried at ambient temperature to form a clear trehalose glass containing iloprost and/or another pharmaceutical agent to be administered in addition to iloprost in solid suspension or solution. The glass is ground to give a powder and remains a free-flowing powder at ambient temperature and humidities. Addition of the powder to water results in the dissolution of the trehalose and the formation of a uniform aqueous suspension of iloprost and/or another pharmaceutical agent to be administered in addition to iloprost.

d) Incorporation of Active in SP Vitreous Delivery Vehicle by Co-Precipitation

20% solutions of trehalose, lactitol, palatinit, GPM or GPS, containing MWPB and iloprost and/or another pharmaceutical agent to be administered in addition to iloprost are dried by spraying into an acetone-solid carbon dioxide freezing bath. The precipitated powders are separated by centrifugation or filtration and air dried to remove residual solvent. The powders again show a monodisperse particle distribution and those containing buffer formulation salts remain dry at ambient temperatures and humidities.

e) Formation of Composite Vitreous Solid Dose Delivery Vehicle of Hydrophobic Active in SP by Drying from Organic Solvents

Two different solvent systems are used to produce composite glasses. In the first case, iloprost and/or another pharmaceutical agent to be administered in addition to iloprost is dissolved in ethanol and an equal volume of water is then added slowly so that the iloprost and/or another pharmaceutical agent to be administered in addition to iloprost which precipitated on each addition is allowed to redissolve. Trehalose is then dissolved in the 50% v/v ethanol solution to a final concentration of 50% w/v. Composite glasses are produced by evaporating the mixed solvent on a hotplate at 70° C. In the second case, iloprost and/or another pharmaceutical agent to be administered in addition to iloprost and trehalose are both dissolved in DMF and again the composite glass is made by evaporation as described above. In both cases, a slightly opalescent glass results. Drops of water are then overlaid on the glass films to study the dissolution and release properties of the glasses.

The results indicate that the glasses behave remarkably differently. Glasses made from DMF are water repellent with an obviously hydrophobic surface. They gradually develop opaque white patches and clumps of precipitated iloprost and/or another pharmaceutical agent to be administered in addition to iloprost where they were in contact with water. Glasses made from 50% ethanol are hydrophilic. They dissolve rapidly in the water and in doing so they release a cloud of very fine particles containing iloprost and/or another pharmaceutical agent to be administered in addition to iloprost. This latter glass appears to contain iloprost and/or another pharmaceutical agent to be administered in addition to iloprost in either a fine solid suspension or a solid solution in the trehalose glass which releases the iloprost and/or another pharmaceutical agent to be administered in addition to iloprost as a precipitate when the trehalose dissolves.

The different behavior of glasses of identical composition after drying from different solvents suggests an interesting and useful process providing precise control over the pattern of deposition of the different glasses during solvent evaporation. Since iloprost is more soluble in DMF than is trehalose, composite glasses of 10-20% iloprost in trehalose prepared from this solvent tend to have hydrophilic trehalose cores and hydrophobic iloprost coatings. In contrast, when 50% ethanol evaporates, the early loss of ethanol in the 97% azeotrope causes iloprost to come out of solution surrounded by trehalose syrup which then solidifies as the continuous phase leading to a iloprost in trehalose glass solid emulsion.

### Example 8

### Protection of Pharmaceutical Agents from Organic Solvents and Temperature Effected by Drying in Trehalose

A solution of iloprost and/or another pharmaceutical agent to be administered in addition to iloprost is dried in an FTS Systems freeze drier with or without 50% trehalose. The drier is used as a vacuum drier and the mixtures dried without freezing. The dried materials are exposed to organic solvents. The contents are redissolved in water, and the activity of the iloprost and/or another pharmaceutical agent to be administered in addition to iloprost is assessed. Iloprost and/or another pharmaceutical agent to be administered in addition to iloprost which is dried with trehalose is more resistant to organic solvents than the sample dried without trehalose.

Iloprost and/or another pharmaceutical agent to be administered in addition to iloprost is freeze-dried in the FTS drier with or without 20% trehalose. The dried iloprost and/or another pharmaceutical agent to be administered in addition to iloprost is stored at room temperature. The sample dried with trehalose exhibits less loss of activity than the sample which was not dried with trehalose.

### Example 9

### Preparation of Vitreous Delivery System with Pharmaceutical Agents Incorporated in Composite SP and/or HDC and/or Carboxylate Glass

a) Coformulation of Vitreous Delivery System of Composite SP and Organic Glasses by Evaporation

Microparticles of trehalose containing MB9 are prepared by spray drying as described in Example 7b. The solution dried contains trehalose and calcium lactate and MB9. These particles are coated by adding them to a saturated solution of zinc palmitate (ZnC₁₆) in toluene and cooling from 60° C. to 30° C. This deposits a layer of ZnC₁₆ on the particles which are then filtered under vacuum to remove the excess ZnC₁₆, washed with acetone and air-dried. The resulting powder remains unwetted in water for at least three days (the particles float in the water without sinking or release MB9 and thereafter slowly release dye into the water). Thus, otherwise water soluble powders may be made water impermeable by coating with metal carboxylates such as ZnC₁₆ to yield slow release formats. Note that the coating material is most likely in crystalline form and not a glass; therefore, the solid phase in which the pharmaceutical agents are suspended need not be in the glass phase to be water impermeable.

b) Coformulation of Vitreous Solid Dose Delivery System of SP Glasses Containing Active and Organic Glasses By Evaporation

A powdered trehalose glass containing iloprost and/or another pharmaceutical agent to be administered in addition to iloprost is added to a mixed carboxylate glass, namely a 1:1 mixture of sodium octanoate and zinc ethylhexanoate, dissolved in an excess of chloroform and evaporated under a stream of N₂ at room temperature to yield a carboxylate glass containing a powder containing the iloprost and/or another pharmaceutical agent to be administered in addition to iloprost in solid suspension or solution. The coformulated glass remains insoluble in water for at least 48 hrs.

c) Coformulation of Vitreous Solid Dose Delivery System of SP Glasses Containing Active and Organic Glasses by Co-Melting

A preformed organic glass formed by quenching a melt of 1:1 mixture of sodium octanoate and zinc ethylhexanoate is softened at 95° C. and a powdered trehalose glass containing iloprost and/or another pharmaceutical agent to be administered in addition to iloprost is added to the melt. The resultant mixture is immediately quenched on an aluminum block precooled to 15° C. A clear carboxylate glass forms containing encapsulated powder containing iloprost and/or another pharmaceutical agent to be administered in addition to iloprost. Varying the nature and ratios of the carbohydrate and organic moieties in the coformulated glasses results in glasses with a range of slow-release characteristics as assessed from their variable dissolution times in water.

d) Coformulation of Vitreous Solid Dose Delivery System of SP Glasses Containing Active and HDC Glasses by Evaporation

The delivery systems are prepared by spray drying using a Buchi B-191 spray drier. Preformulated spray-dried trehalose/MB9 dye is suspended in a solution of TOAC (4 g) and azobenzene (0.029 g) in dichloromethane (100 ml) and spray drier at an inlet temperature of 40° C. A powder is obtained with the TOAC glass The composite delivery vehicle shows delayed release of the iloprost and/or another pharmaceutical agent to be administered in addition to iloprost when immersed in an aqueous solution.

e) Coformulation of Vitreous Solid Dose Delivery System of SP Glasses Containing Active and Plastics by Evaporation

A powdered trehalose glass containing iloprost and/or another pharmaceutical agent to be administered in addition to iloprost is added to a solution of perspex dissolved in an excess of chloroform and evaporated under a stream of N₂ at room temperature to yield a solid perspex block containing the powder containing iloprost and/or another pharmaceutical agent to be administered in addition to iloprost in solid solution.

### Example 10

### Preparation of Solid Dose Delivery Systems of Organic Glasses by Evaporation

a) Preparation of Carboxylate Solid Dose Delivery Systems by Solvent Evaporation

Aluminum hexanoate is dissolved in chloroform (0.5 g/10 ml) together with a fine suspension of 1 wt % MB9 as a tracer dye. A fine amorphous film (100-200 μm thickness) is formed by casting on silicate glass slides and evaporating off the solvent in a warm air-stream. Release of dye into distilled water is monitored over 5 hr. No devitrification of these glasses is observed and the films remain transparent, though they decolorize as the dye diffused out into medium.

Amorphous films are also formed from calcium neodecanoate dissolved in chloroform (0.5 g/10 ml) as described above. Release of dye from these thicker (1-2 nm thickness) films into distilled water is again monitored over 24 hr. In contrast to the Aluminum films, dye release from the calcium neodecanoate films follows the dissolution of the films as monitored by atomic adsorption spectroscopy of Ca²⁺.

b) Preparation of Composite Vitreous Solid Dose Delivery Systems of SP Glass Containing Active Incorporated into Carboxylate Glass by Evaporation

Films of glucose glass incorporating 1 wt % MB9 are formulated by quenching from the melt. These films are coated with thin (100 μm thickness) amorphous metal carboxylate films by evaporation of solution of the carboxylate in chloroform (0.5 g/10 ml). The metal carboxylates used are aluminum hexanoate and octanoate, calcium neodecanoate and magnesium isostearate and neodecanoate. Dissolution of the films is monitored by release of dye into distilled water. These delivery systems delayed dye release for times ranging from minutes to hours, except for those formed from magnesium isostearate which delays release of dye for 10 days.

### Example 11

### Preparation of HDC Solid Dose Systems

Several HDC glasses are prepared by melting and quenching. In the following Examples, the component HDCs are purchased from Aldrich Chemicals with the exception of TOPR which is synthesized according to the method described by Akoh et al. (1987). The components form glasses with little if any decomposition. The fructose, sucrose and to some extent, glucose, melt with noticeable decomposition or polymerization. An ester such as α-D-glucose pentaacetate is stable at its melting point and forms a clear colorless glass as it is being quenched. The greater stability of the ether and ester derivatives is clearly an advantage in the encapsulation of reactive materials.

The HDCs with particularly low melting points form soft waxy glasses after being quenched. The nmr spectrum of vitreous α-D-glucose pentaacetate is identical to that of crystallized α-D-glucose pentaacetate.

The glass formed from β-D-glucose pentaacetate is poorly soluble in water and a disc (20 mm diameter and 2.5 mm thick) prepared from this ester placed in flowing water lost about 33% of its original weight in 10 days. Another glass disc of similar dimensions is prepared from α-D-glucose pentaacetate and placed in 1 l of water, which is replaced daily. After 7 days, the glass loses 20% of its original weight. The rate of release of encapsulated Acid Blue dye from this glass is quite constant. The release rate of the dye is higher in the first day as the release happens mainly from the surface of the glass disc.

Excellent recoveries are obtained in the encapsulation of several organic substances in the glasses. Glass discs of α-D-Glucose pentaacetate containing 2% w/w of the materials listed in Table 4 are formed by melting and quenching and then ground. Photochrome II is 5-chloro-1,3-dihydro-1,3,3-trimethyl spiro[2H-indole-2,3′-[3H]-napth[2,1-b][1,4]-oxazine. The encapsulated materials are extracted by the suitable solvent such as methanol or water. The results are depicted in Table 4.

TABLE 4

| Encapsulated material | b.p. ° C. | m.p. ° C. | Application |
|---|---|---|---|
| Acid yellow 65 | | >300 | Water |
| | | | soluble dye |
| Acid blue 129 | | >300 | Water |
| | | | soluble dye |
| Disperse red 1 | | 161 | Non-linear |
| | | | optical |
| | | | material |
| Mordant blue 9 | | >300 | Water |
| | | | soluble dye |
| Ethyl hexanoate | 168 | | |
| Ethyl octanoate | 207 | | |
| Oxadiazon | | 90 | Pesticide |
| Azobenzene | 293 | | |
| Melatonin | | 117 | veterinary |
| Photochrome II | | 183 | hormone |
| | | | Photochrome |

The rates of release of Acid Blue 129 depend on the dissolution rates and shapes of the glasses.

### Example 12

### Formation and Release Properties of Vitreous HDC Delivery Systems by Quenching from the Melt

a) Formation and Release Properties of Simple and Composite Vitreous HDC Glasses from the Melt

In the following experiments, the delivery system is preformulated, whether as a single material, or as a mixed composition. This is carried out by intimately grinding the component HDCs together, followed by careful, controlled melting in a furnace, between 120-140° C. and with normal atmosphere to form melts. The melts are quenched to glass by pouring over a brass block. This glass is then finely ground.

To assess the release characteristics of the composition, MB9 dye (1 or 5 wt %) is mixed with the ground glass prior to re-melting at 140° C. The melt is quenched to form small glass beads (2.5 mm diameter) which are used in controlled release experiments.

Controlled release of encapsulated dye is monitored by suspending three such beads in 25 or 50 ml of deionized water or PBS solution at ambient temperatures (27-30° C.) or at 37° C., as indicated. The media are undisturbed, except for periodic stirring and are replaced at set intervals with fresh media (generally at 72 hr intervals). Both single HDC glasses and composite HDC glasses are formed. Dye release is measured by Spectrophotometry (516 nm λmax). U.S. Pat. No. 6,586,006, the disclosure of which is incorporated herein by reference in its entirety, describes the release characteristics of various HDC compositions.

b) Incorporation of Pharmaceutical Agents in HDC by Quenching from the Melt

The compatibility of iloprost and/or another pharmaceutical agent to be administered in addition to iloprost with glass may be assessed as follows. TOAC is pre-melted at 150° C., before being quenched to glass. The glass is finely ground with iloprost and/or another pharmaceutical agent to be administered in addition to iloprost before being remelted. The clear melt is again quenched to yield the composite HDC/active glass. Thermal analysis is carried out on a Rheometric Scientific Differential Scanning Calorimeter (DSC) at a heating rate of 10°/min under a nitrogen atmosphere. Samples containing TOAC/Iloprost and/or another pharmaceutical agent, TOAC/iloprost and/or another pharmaceutical agent plus MB, TOAC alone or TOAC/MB9 are prepared.

Release characteristics of the vitreous HDC solid dose delivery systems is studied by monitoring the release of MB9 from glasses containing TOAC/iloprost and/or another pharmaceutical agent. For analysis of stability of iloprost and/or another pharmaceutical agent to be administered in addition to iloprost in the vitreous HDC solid dose delivery systems, Iloprost and/or the other pharmaceutical agent is recovered from the samples by dissolving the glass in acetonitrile and analyzed by HPLC.

### Example 13

### Formation of Vitreous HDC Solid Dose Delivery Systems by Evaporation of Solvent

a) Formation of HDC Glasses by Solvent Evaporation

As described above, TOAC makes a good delivery vehicle by quenching from the melt. Such a delivery system has a low melting point and very little tendency to recrystallize.

Dichloromethane (DCM) and chloroform are standard solvents for TOAC, which is also soluble in other solvents such as acetonitrile. Glasses are made by evaporating DCM on a hotplate set at 65° C. from a 25% solutions of TOAC (50% solutions often deposited crystals in the pipette tip). Drying is carried out for 2 hr to be certain of complete dryness. Uniform glasses are produced by using an Eppendorf-type pipette to deliver 100 μl to a slide recently placed on the hotplate and then removing about 50 μl by using the clear/expel volume of the pipette. Glasses are very clear and adherent when first made but gradually recrystallize over 1 month at room temperature (RT) and 50-60% relative humidity (RRH).

Trehalose glasses similarly made by evaporating water from a 50% trehalose solution are clear when first formed but gradually recrystallize over a period of several weeks.

b) Incorporation of Pharmaceutical Agents into HDC Glasses by Solvent Evaporation: Powders Suitable for By-Inhalation

Iloprost and/or another pharmaceutical agent to be administered in addition to iloprost is incorporated into a TOAC glass by dissolving both crystalline TOAC and iloprost and/or another pharmaceutical agent to be administered in addition to iloprost in DCM and evaporating the solvent at 70° C. on a hotplate. These glasses are perfectly water clear and transparent and are resistant to changes in glass structure. However, when immersed in liquid water, the surface of the glass slowly recrystallizes so that microscopic pyramidal crystals of TOAC can be seen under an inverted microscope. Iloprost and/or another pharmaceutical agent to be administered in addition to iloprost previously incorporated in the glassy TOAC matrix is now released into the liquid phase.

c) Incorporation of Active into HDC Glasses by Solvent Evaporation; Spray Dried Powders Suitable for By-Inhalation

Studies were performed using iloprost and/or another pharmaceutical agent to be administered in addition to iloprost dissolved in DCM. The solution is spray dried in a Buchi B-191 spray drier, using an inlet temperature of 40° C. This results in a powder, that contains the iloprost and/or another pharmaceutical agent to be administered in addition to iloprost.

For analysis the iloprost and/or another pharmaceutical agent to be administered in addition to iloprost is extracted from the spray-dried powder by dissolving the powder and prior to analysis by HPLC. On analysis by HPLC, it is concluded that the iloprost and/or another pharmaceutical agent to be administered in addition to iloprost is effectively being released into aqueous solution. Bioavailability of the iloprost and/or another pharmaceutical agent to be administered in addition to iloprost from the delivery system is tested by immersion in an aqueous solution for a short time. Stability of the iloprost and/or another pharmaceutical agent to be administered in addition to iloprost in the spray-dried formulation is tested at high humidity and elevated temperature. The results indicate a resistance to high humidity and elevated temperature, stability in the glass and ready bioavailability in vitro tests.

d) Formation of Vitreous Solid Dose Delivery Vehicles of Composite HDC Glasses by Solvent Evaporation

In addition to TOAC, two other hydrophobically modified saccharides, α-GPAC and TOPR, may be used in mixtures to provide mixed glasses with desired properties.

Mixed glasses of pairs of these HDCs are produced by mixing the crystalline components in various proportions and then producing glasses either by evaporation of the solvent DCM on a hotplate or by melting at 150° C. and quenching on a brass plate.

The resulting glasses are tested for their utility as controlled release matrices in two ways. First, they are assessed for their ability to resist devitrification on exposure to high RH at RT. Second, they are immersed in water or phosphate-buffered saline (PBS) to study their solubility and rate of erosion by surface recrystallization.

Single component glasses of both α- and β-GPAC can only be made by quenching from the melt. When solvent evaporated, solutions of this HDC always crystallize. Single component glasses of TOAC and TOPR are readily produced by either solvent evaporation or quenching but are very susceptible to devitrification at high RH, showing complete recrystallization of thin glass films on microscope slides and surface recrystallization of quenched disks at RH from 75% to 95% after overnight exposure. The mixed glasses behave as described in Table 5.

TABLE 5

| % GPAC | % TOAC | % TOPR | Initial Form | After RH 24 hr |
|---|---|---|---|---|
| | 100 | | Glass | Cryst + + + + |
| 10 | 90 | | Glass | Glass |
| | 90 | 10 | Glass | Glass |
| 50 | 50 | | Glass | Glass |
| 90 | 10 | | Cryst + + + + | ND |
| 80 | 20 | | Cryst + | Cryst + + + + |
| 90 | | 10 | Cryst + + + + | ND |

The effect of different RHs is very uniform. While the pure TOAC and some of the composite glasses crystallize at all RHs from 75% to 95%, the other composite glasses remain amorphous at all the RHs studied.

The 10% α-GPAC and 10% TOPR in TOAC glasses and the 50:50 molar ration TOAC:α-GPAC glass are also immersed in water to examine their rate of devitrification in liquid water rather than humid air. The first glass recrystallizes within 20-30 min while the second develops a few small crystals after 4 hr while the 50:50 glass does not change over 4 days indicating surprisingly low solubility.

As a vehicle for powder delivery of drugs to the deep lung, the 10% α-GPAC in TOAC glass shows the very desirable properties of resistance to 95% RH such as might be experienced in an inhaler and in the air passages with, at the same time, rapid recrystallization in liquid water such as in the fluid layer lining the alveolae.

Glasses of TOAC with or without the addition of 10% or more of α-glucose pentaacetate or trehalose octapropanoate provide a range of resistance to ambient RH and of solubility rates allowing a degree of tailoring of the controlled release of drugs dispersed in such glasses.

e) Incorporation of Pharmaceutical Agents into Composite Slow Release HDC and/or SP Glasses by Solvent Evaporation

For maximum utility, the slow release characteristics of HDCs should be usable with both hydrophobic and hydrophilic molecules. The former are readily prepared in solid solution in one of the HDCs either by solvent evaporation or by direct dissolution in the melt followed by quenching. Hydrophilic molecules are not directly soluble in HDCs.

The disclosed HDC may be used to incorporate hydrophilic substances in a very uniform and useful distribution in a matrix of HDCs. The process is well illustrated by using trehalose as the hydrophilic substance and TOAC as the hydrophobic matrix. Good solvents for both modified and native trehalose are DMF and DMSO. When a solution of 10% trehalose and 90% TOAC in DMF is evaporated to dryness, a glass with a frosted or opalescent appearance results. Under the microscope, this is seen to be a very uniform distribution of spherical glassy microbeads of uniform size in a continuous matrix (FIGS. 16 and 17). By rough measurement with an eyepiece graticule, the size of the microbeads is about 4 micrometers in diameter.

The identity of the 2 phases may be verified by incorporating a small quantity of the intensely hydrophobic lipid dye, Oil Red O together with a small quantity of the hydrophilic dye, Methylene Green in the solution in DMF before making the glass. The hydrophobic Oil Red O partitions exclusively into the continuous phase, revealing it to be TOAC, whereas the hydrophilic Methylene Green partitions exclusively into the discontinuous uniform particles revealing them to be trehalose (FIG. 18). The composite glass thus formed consists of a very uniform and stable glass in glass “solid emulsion” or “solid suspension” rather than solid solutions such as are seen with the hydrophobic guest substances XPDO, CSA or Oil Red O.

When the same mixtures of trehalose and TOAC is evaporated from solution in DMSO, the appearance of the composite glass is different. In this case, the glass is more transparent and under the microscope the discontinuous trehalose phase is in 2 forms. One form is a very fine dispersion of extremely small trehalose particles uniformly dispersed throughout the continuous matrix. The other form consists of larger spherical beads of trehalose concentrated in a cluster in the center of the composite glass.

Without wishing to be bound by any one theory, it seems likely that the different patterns found reflect differences in the solubility of the two carbohydrates in the solvents used so that their deposit from solution occurred at different stages of the evaporation of the solvent. Confirmation of this explanation may be provided with experiments to produce composite glasses in the opposite orientation i.e. with a hydrophobic guest substance dispersed finely in a hydrophilic continuous matrix.

g) Toxicity of HDC Glasses

A saturated solution of TOAC in deionized distilled water (0.42 g in 20 mls) is tested for toxicity in vitro using the African Green monkey kidney-derived cell line Vero, in either a 10-fold serial dilution or by adding the TOAC powder directly to the tissue culture medium. No toxic effects are observed in the week of culture and cell division is normal.

### Example 14

### Release of Model Pharmaceutical Agent from Single HDC Solid Dose Form into Surface Active Mucosal Milieu Mimic

The release characteristics of a particular HDC formulation into a surface active mucosal milieu may be assessed as follows. Dye disperse red (DR1) is incorporated into the formulation to be assessed and the formulation is introduced into a detergent containing medium [3% (w/v) sodium dodecyl sulphate in 0.9% saline solution] to mimic a surface active mucosal milieu. The release rate of dye from the formulation into the mucosal milieu mimic is assessed in USP (vol. 23) type 2 dissolution studies, using a Distek (Model 2100) dissolution system and the dye released is quantitated spectrophotometrically at 502 nm using a Hewlett-Packard (Model 8453) diode array spectrophotometer.

### Example 15

### Release of Model Pharmaceutical Agent from Mixed HDCs Solid Dosage Form into Surface Active Mucosal Milieu Mimic

The release characteristics of formulations comprising more than one HDC may be assessed using the in vitro model as described in Example 14. A composition L containing a desired combination of more than one HDC and the DR1 dye is prepared. The release of DR1 from the solid dose form into a detergent containing medium [3% (w/v) sodium dodecyl sulphate in 0.9% saline solution] to mimic a surface active mucosal milieu is determined as described in Example 14.

### Example 16

### Release of Hydrophobic Pharmaceutical Agent from HDC Solid Dose Form into Surface Active Mucosal Milieu Mimic

The bioavailability of a hydrophobic pharmaceutical agent may be assessed in the in vitro model described in Example 14. A hydrophobic pharmaceutical agent is incorporated into a formulation comprising the HDC using any of the techniques described above. The release characteristics are assessed using the in vitro assay of Example 14 and an appropriate assay for the pharmaceutical agent.

### Example 17

### Effect of Incorporated Surface Active Agent on Release of Model Hydrophobic Pharmaceutical Agent from HDC Solid Dose Form

The effect of incorporated surfactant on release of hydrophobic model pharmaceutical agent from a solid dose form may be tested using Oil Red O as a model hydrophobic pharmaceutical agent incorporated in a solid dose form comprising an HDC and a surfactant. Basically, 1% (w/w) of the hydrophobic dye oil red O (ORO) is mixed with 10-40% (w/w) surfactant and the desired HDC using any of the methods described above. The release of dye from the solid dose form is assessed using an in vitro USP (volume 23) type 2 dissolution test in 0.1M HC1 as the dissolution medium. The USP 2 dissolution apparatus containing 900 ml 0.1M HC1 at 37° C. is stirred at 100 rpm and samples of approximately 5 ml taken hourly and assayed by UV spectroscopy at 523 mm in 10 mm cell against a 0.1M HC1 reference cell. The assay data are corrected for ongoing media loss during the test.

### Example 18

### Bioavailability Study of Pulmonary Delivery of a Pharmaceutical Agent In Vivo

An HDC formulation comprising iloprost and/or another pharmaceutical agent to be administered in addition to iloprost is prepared using any of the methods described above. Particles having the desired dimensions are prepared and administered as a dry powder solid dose form to the lungs of dogs or pigs. Adsorption of iloprost and/or another pharmaceutical agent to be administered in addition to iloprost is analyzed by chromatographic assay for iloprost and/or another pharmaceutical agent to be administered in addition to iloprost in the blood of the animals at suitable time intervals. The solid dose forms show enhanced pulmonary bioavailability compared to controls of iloprost and/or another pharmaceutical agent to be administered in addition to iloprost administered in lactose formulations.

### Example 19

### Synthesis and Physical Properties of Derivatized Carbohydrates

Carbohydrate derivatives may be routinely synthesized by standard esterification of the carbohydrate with the chloride of the desired hydrocarbon side chain under anhydrous conditions and the resulting derivatives purified by standard techniques of solvent extraction and re-crystallization. For example, trehalose octa-3,3-dimethylbutyrate may be synthesized by reacting 3,3,-dimethylbutyroyl chloride with trehalose in anhydrous pyridine, followed by extraction with ether, hydrochloric acid, potassium carbonate solution and water and finally re-crystallized twice from alcohol to yield colorless, needle-like crystals (˜80% yield) of m.pt 138-140° C., α_{D} 112°. Trehalose hexa-3,3-dimethylbutyrate (THEX) can be prepared by protecting the 6,6′-hydroxy group of trehalose with a bulky group such as trityl or t-butyldiphenylsilyl, for example by heating trehalose and trityl chloride in pyridine. The 6,6′-ditrityltrehalose can be acylated with 3,3-dimethylbutyroyl chloride in pyridine to give 6,6′-ditritylhexa-3,3-dimethylbutyryltrehalose. The trityl group can be removed by strong acid, for example hydrogen bromide in acetic acid, to give THEX. TACT can be prepared by acylating THEX with acetic anhydride in pyridine. Suitable work-up yields the HDC in crystalline form. The physical characteristics, melting points and glass transition temperatures (Tg, ° C.) of selected carbohydrate derivatives are shown in Tables 6-10.

Table 6 shows examples of fully substituted pivalate and tertbutyl acetate derivatives which show Tgs of up to 81° C., much higher than the equivalent straight chain derivatives (butyrate and valerate) which form oily syrups instead of glassy solids. This unusual property of branched-chain derivatives enables more hydrophobic derivatives (compared to the acetates) to be prepared, which permits further reduction of pharmaceutical agent release rates for longer term applications.

Table 7 illustrates that mixed straight and branched chain ester derivatives of trehalose result in glasses with lower water solubilities, yet useful high Tgs. Interestingly, several of these derivatives fail to crystallize during the purification steps, thus illustrating that selected mixed ester derivatives can be difficult to crystallize. Preferred derivatives are those that form stable hydrophobic glasses with high Tgs (greater than about 40° C.), yet have a degree of instability that produces a defined, even crystal growth as the HDC glass interacts with water. The mixed ester derivatives offer a combination of both glass stability and increased hydrophobicity, which are useful to delay release of pharmaceutical agents.

Partially substituted trehalose derivatives, as shown in Table 8, show surprising characteristics of very high Tgs, and in some cases a reluctance to also crystallize. These derivatives also fail to crystallize when immersed in water. For example, trehalose hexa-3,3-dimethylbutyrate (THEX) is stable in the glassy state when immersed in saline medium at 37° C. When the hydroxyl groups are replaced with acetates, as with trehalose diacetate hexa 3,3-dimethylbutyrate (TACT), the glass stability is reduced, though both these glasses are more stable than trehalose octa 3,3 dimethylbutyrate (TOCT). These compounds are thus useful for controlling the release rate of drugs formulated within the respective glasses. To extend this, blends of two or more HDCs permit further variations in controlling the rate of devitrification and hence release of pharmaceutical agents. For example, the pure α,β anomers of lactose isobutyrate heptaacetate crystallize from solution; however, when a small amount of the corresponding anomer is added, the blend fails to crystallize. Thus, using combinations of HDCs and/or anomers thereof, the rate of drug release can be controlled.

Table 9 illustrates some selected properties of other disaccharide ester derivatives Cellobiose octaisobutyrate has a surprisingly high melting temperature, yet is very hard to quench to glass. Lactose and cellobiose derivatives tend to have higher Tgs than trehalose and sucrose derivatives. Lactose derivatives devitrify much more slowly than their corresponding trehalose derivatives despite their similar Tgs. For example, lactose isobutyrate heptaacetate is very stable in the glassy state, when immersed in water. It also has a very high Tg (Table 9).

TABLE 6
Effect of branched versus straight chains

| Derivatized | | | |
|---|---|---|---|
| Carbohydrate | M.P.(° C.) | Tg(° C.) | COMMENT |
| Trehalose | 135.9 | 55 | C2 straight chain |
| octaacetate | | | |
| Trehalose | 47 | 3 | C3 straight chain. Glass not |
| octapropionate | | | stable above ambient |
| | | | temperature |
| Trehalose | syrup | syrup | C4 straight chain. No glass |
| octabutyrate | | | formation |
| Trehalose | 78 | 7 | C4 branched chain. Glass |
| octaisobutyrate | | | formed, but not stable above |
| | | | ambient temperature |
| Trehalose | syrup | syrup | C5 straight chain. No glass |
| octavalerate | | | formation |
| Trehalose | 188 | 81 | C5 branched chain. Glass |
| octapivalate | | | formed now stable above |
| | | | ambient temperature |

TABLE 7
Formation of mixed branched and straight chain derivatives

| Derivatized | | | |
|---|---|---|---|
| Carbohydrate | M.P.(° C.) | Tg(° C.) | COMMENT |
| Trehalose 6,6′di-(2,2- | amorphous | 47 | Material not |
| dimethylbutyrate) hexaacetate | | | isolated in |
| | | | crystalline form |
| Trehalose 6,6′-di-(3,3- | 165 | 50 | |
| dimethylbutyrate) hexaacetate | | | |
| Trehalose 6,6′-diaacetate | 140 | 44 | |
| hexa-3,3-dimethylbutyrate | | | |
| Trehalose 6,6′-di-(2- | 63 | 30 | |
| ethylbutyrate) hexaacetate | | | |
| Trehalose 6,6′-diisobutyrate | 87 | 42 | |
| hexaacetate | | | |
| Trehalose 4,4′-diisobutyrate | 123 | 41 | |
| hexaacetate | | | |
| Trehalose 6,6′-dipropionate | amorphous | 43 | Material not |
| hexaactetate | | | isolated in |
| | | | crystalline form |
| Trehalose 4,4′-dipropionate | amorphous | 42 | Material not |
| hexaactetate | | | isolated in |
| | | | crystalline form |
| Trehalose 6,6′ dipivalate | 159 | 56 | |
| hexaacetate | | | |

TABLE 8
Effect of partially derivatization with branched chains

| Derivatized | M.P. | Tg | |
|---|---|---|---|
| Carbohydrate | (° C.) | (° C.) | COMMENT |
| Trehalose octapivalate | 188 | 81 | Very hydrophobic, most |
| | | | resistant to devitrification |
| | | | in aqueous environment |
| Trehalose heptapivalate | 182 | 73 | |
| Trehalose hexapivalate | 203 | 86 | |
| Trehalose pentapivalate | amorphous | 81 | Material not isolated in |
| | | | crystalline form |
| Trehalose tetrapivalate | 301 | 96 | Most hydrophilic, least |
| | | | resistant to devitrification |
| | | | in aqueous environment |
| Trehalose octa-3,3- | 139 | 42 | |
| dimethylbutyrate | | | |
| Trehalose hexa-3,3- | amorphous | 64 | Material not isolated in |
| dimethylbutyrate | | | crystalline form |
| Trehalose tetra-3,3- | 237 | 82 | |
| dimethylbutyrate | | | |

TABLE 9
Effect of changing carbohydrate backbone

| Derivatized | | | |
|---|---|---|---|
| Carbohydrate | M.P.(° C.) | Tg(° C.) | COMMENT |
| α,β-Lactose | 147 | 67 | Undefined anomeric ratio |
| octaacetate | | | |
| α-Lactose | 119 | 70 | |
| octaacetate | | | |
| β-Lactose | 87 | 63 | |
| octaacetate | | | |
| Lactose isobutyrate | amorphous | 60 | 1:1 ratio of α and β |
| heptaacetate | | | anomers |
| β-Lactose | amorphous | 60 | Mixed straight and |
| isobutyrate | | | branched chain derivative |
| heptaacetate | | | |
| α-Lactose 3-acetyl- | 128 | 48 | Mixed straight and |
| hepta-3,3- | | | branched chain derivative |
| dimethylbutyrate | | | |
| α-Lactose octa-3,3- | 149 | 53 | C5 branched chain. Glass |
| dimethyl-butyrate | | | stable above ambient |
| | | | temperature |
| β-Lactose | 168 | 88 | C5 branched chain. Glass |
| octapivalate | | | stable well above |
| | | | ambient temperature |
| α-Cellobiose | 224 | 65 | Poor glass former |
| octaacetate | | | |
| β-Cellobiose | 193 | 53 | Good glass former |
| octaacetate | | | |
| β-Cellobiose methyl | 138 | 77 | Mixed straight chains |
| heptaacetate | | | derivative |
| β-Cellobiose ethyl | 182 | 52 | Longer straight chain |
| heptaacetate | | | (C2) gives lowers Tg |
| β-Cellobiose | syrup | 15 | C3 straight chain. Glass |
| octapropionate | | | not stable above ambient |
| | | | temperature |
| Raffinose undeca- | 83 | 15 | Branched chain |
| isobutyrate | | | derivative of |
| | | | trisaccharide |

### Example 20

### Incorporation of Pharmaceutical Agents into Single and Composite Formulations of Derivatized Carbohydrates and Controlled Release In Vitro

a. Formulation and Controlled Release of a Model Hydrophilic Drug

A desired hydrophilic pharmaceutical agent to be administered in addition to iloprost is loaded into formulations comprising a desired HDC, combination of HDCs, or one or more HDCs plus a surfactant. Release of the hydrophilic pharmaceutical agent from the HDC solid dose delivery vehicle is assessed using an in vitro USP (volume 23) type 2 dissolution test in saline containing 0.1% sodium cholate as the dissolution medium. Tg measurements for each formulation are also conducted.

b. Formulation and Controlled Release of Iloprost

Iloprost is loaded at a desired level (for example between about 0.01%-about 30%) into formulations comprising a desired HDC, combination of HDCs, one or more HDCs or one or more HDC's plus one or more surfactants. Melt incorporation is carried out by melting the HDCs at 150-170° C. and dissolving the active in the melt at 120-140° C. Rotary evaporation is carried out using a Buchi Rotavapor R-124. Release rates and Tg's are determined for each formulation.

### Example 21

### Synthesis and Characterization of Glycoside of Sugar Alcohol Derivates

Acetyl derivatives of polyols are prepared, wherein the polyols are the following glycosides of sugar alcohols: lactitol, palatinit, and the individual sugar components of palatinit, as described below.

10 g of the polyol is dissolved in 40 mL of acetic anhydride containing 4 g F of sodium acetate. When all the sugar has dissolved, 100 mL of distilled water is added to the solution and the mixture is extracted with dichloromethane to extract the derivatized polyol. The acetylated polyol is recovered by evaporating off the solvent and the derivative is characterized by nuclear magnetic resonance spectroscopy (NMR) and differential scanning calorimetry (DSC). The products are obtained and characterized by NMR and DSC, for the acetyl derivatives of lactitol (4-O-β-D-galactopyranosyl-D-glucitol), palatinit [a mixture of GPS (α-D-glucopyranosyl-1→6-sorbitol) and GPM (α-D-glucopyranosyl-1→6-mannitol)], and its individual sugar alcohol components GPS and GPM. Table 10 shows the melting points and Tgs (glass transition state temperatures) for the acetyl derivatives formed, lactitol nonaacetate, palatinit nonaacetate and GPS nonaacetate and GPM nonaacetate.

The glasses show a range of melt temperatures suitable for the incorporation of labile substances such as iloprost and/or another pharmaceutical agent to be administered in addition to iloprost, without thermal degradation.

TABLE 10

| | Melting Point | |
|---|---|---|
| Derivative | (° C.) | Tg (° C.) |
| Lactitol nonaacetate | 119 | 39.5 |
| Palatinit nonaacetate | 108 | 35.3 |
| GPS nonaacetate | 204 | 17.4 |
| GPM nonaacetate | 87 | 35 |

### Example 22

### Formation of Glasses Using Derivatives of Glycosides of Sugar Alcohols and Analysis of Their Solvent Properties

Glasses are formed of the derivatives of glycosides of sugar alcohols prepared as described above in Example 21 by quenching from the melt according to the method described in PCT GB95/01861, the disclosure of which is incorporated herein by reference in its entirety. Various dyes are added to the melts and then mixed before quenching to form glasses incorporating the dyes. Solubility of the dyes in the melt and in the quenched glass is assessed visually as an increase in dye intensity and the presence of particulate material. The solubility of the dyes in lactitol nonaacetate glasses is shown in Table 11; these glasses are also characterized by DSC. The glasses are found to be good solvents for poorly water soluble solutes. Incorporation of such active substances showed little effect on the Tg of the glasses formed as assessed by DSC and no evidence of devitrification is observed even after 2 months at ambient temperature and humidity. The glasses are thus suitable for the encapsulation and controlled release of iloprost and/or another pharmaceutical agent to be administered in addition to iloprost.

TABLE 11

| Dye | Water Solubility | glass solubility |
|---|---|---|
| Napthol green B | ± | ± |
| Mordant blue 9 | + | − |
| Acid yellow 65 | ++ | −− |
| Disperse red 1 | −− | +++ |

### Example 23

### Formation of Glassy Matrix of Modified Glycosides Containing Pharmaceutical Agents for Controlled Release

Iloprost and/or another pharmaceutical agent to be administered in addition to iloprost are incorporated in a glass of lactitol nonaacetate in a desired amount. For example, the iloprost and/or another pharmaceutical agent to be administered in addition to iloprost may be incorporated at a concentration of between about 0.1% and about 10% by weight. The iloprost and/or another pharmaceutical agent to be administered in addition to iloprost may be incorporated by either quenching from the melt or evaporation from solvent. For glass formation by quenching from the melt, the iloprost is dissolved in the desired modified glycoside at 120° C. and the mix quenched immediately after it went clear. For glass formation by solvent evaporation, the iloprost and/or another pharmaceutical agent to be administered in addition to iloprost are dissolved in an appropriate solvent and the solvent is evaporated off in an air stream. The glasses formed by both methods are optically clear and remain clear on storage at ambient temperatures and humidities for at least a month. The Tgs of the glasses are approximately 39° C.

### Example 24

### Controlled Release of Active Molecules Dissolved in Modified Glycosides

To assess the release characteristics of formulations comprising modified glycosides, disperse Red 1 is incorporated as a model compound in a glass comprising one or more modified glycosides by melt mixing. The release of model active from 0.5 mm and 3 mm thickness layers of glass, into either water or an aqueous solution of 5% Tween 20, is monitored by absorbance at 510 nm.

### Example 25

### Assessment of Stability of Iloprost Formulations

Iloprost Content Assay

To assay iloprost formulation for content and impurities, 50 mg formulation is placed into a 50 ml volumetric flask and 20 ml acetonitrile/water (50/50, v/v) is added to dissolve the solution. If necessary, the solution may be warmed to facilitate dissolution. 20 ml PBS 7.4 is added and the solution is mixed before diluting to volume. The resulting solution is Solution A. The solution is filtered through a 0.2 μm PTFE filter before assay by HPLC as described below.

Iloprost Related Substance Assay

0.5 ml of the above iloprost solution is transferred into a 50 ml volumetric flask. 20 ml acetonitrile/water (50/50,v/v) and 20 ml PBS 7.4 are added. The solution is mixed and diluted to 50 ml with PBS 7.4 before assay by HPLC. The resulting solution is Solution B.

Individual iloprost related peaks detected from Solution A are integrated. The relative percentage of the iloprost related peaks is calculated by comparing the peak areas to the area of the main peaks integrated from Solution B.

A formulation containing 4.0 μg/ml, 1.33 mg/ml TR153, 0.13 mg/ml trehalose and 0.13 mg/ml DPPGNa was prepared as described above. Three samples of this formulation were stored at 4° C. for 8 days. Stability of the iloprost (both isomer 1 and isomer 2) was assessed by weighing 100 mg formulation into a 100 ml volumetric flask. 40 ml acetonitrile/PBS 7.4 (50/50, v/v) was added to dissolve the formulation and the mixture was dilute to a volume of 100 ml using PBS 7.4. The solution was filtered and analyzed by HPLC using the protocol below.
Injection volume: 400 μl
Detector: UV detector, 200 nm
Column: Spherisorb ODS2, 3 μm, steel 125 mm×4.6 mm
Mobile phase: See below for preparation
Flow rate: 1 ml/minute
Column temperature: 20° C.
Auto sampler temperature: 18° C.
Run time: 30 minutes
Mobile phase preparation: Dissolve 8 g β-cyclodextrin in 670 ml water for chromatography then pass through a 5.0 cm diameter regenerated cellulose membrane filter, pore size</=0.2 μm (Sartorius, 18407-50-N). After adding 330 ml acetonitrile, adjust the pH to 2.0 with phosphoric acid (84-90%), then degas for at least 5 minutes by sonication.

As shown in Table 12, the preparation was stable when stored at 4° C. for 8 days.

TABLE 12

| | Day 1 | | | | | | | | |
|---|---|---|---|---|---|---|---|---|---|
| Con | Iloprost 1 | | | Iloprost 2 | | | Total | | |
| (4 ug/ml) | Ind. | Mean 1 | Con 1 | Ind. | Mean 2 | Con 2 | Ind. | Mean | Con |
| Std | 1654738 | 1657264 | | 1499379 | 1501644 | | 3154117 | 3158908 | |
| Sample 1 | 1680363 | 1686005 | 4.056 | 1525050 | 1534058 | 4.062 | 3205413 | 3220062 | 4.059 |
| Sample 2 | 1698124 | | 4.099 | 1546267 | | 4.119 | 3244391 | | 4.108 |
| Sample 3 | 1679527 | | 4.054 | 1530856 | | 4.078 | 3210383 | | 4.065 |
| Std | 1659790 | | | 1503908 | | | 3163698 | | |

| Con | Change in P | | | Change in Con | | |
|---|---|---|---|---|---|---|
| (4 ug/ml) | Iloprost 1 | Iloprost 2 | Total | Iloprost 1 | Iloprost 2 | Total |
| Std | | | | | | |
| Sample 1 | −0.52 | 0.03 | −0.26 | −0.81 | −0.42 | −0.62 |
| Sample 2 | −0.42 | −0.06 | −0.25 | −0.71 | −0.51 | −0.61 |
| Sample 3 | 0.35 | 0.57 | 0.46 | 0.06 | 0.13 | 0.09 |
| Std | | | | | | |

| | Day 8 | | | | | | | | |
|---|---|---|---|---|---|---|---|---|---|
| Con | Iloprost 1 | | | Iloprost 2 | | | Total | | |
| (4 ug/ml) | Ind. | Mean 1 | | Ind. | Mean 2 | | Ind. | Mean | Con |
| Std | 1652805 | 1662088 | | 1497575 | 1508338 | | 3150380 | 3170426 | |
| Sample 1 | 1671596 | 1682686 | 4.023 | 1525486 | 1536789 | 4.045 | 3197082 | 3219475 | 4.034 |
| Sample 2 | 1690993 | | 4.070 | 1545264 | | 4.098 | 3236257 | | 4.083 |
| Sample 3 | 1685468 | | 4.056 | 1539618 | | 4.083 | 3225086 | | 4.069 |
| Std | 1671371 | | | 1519101 | | | 3190472 | | |

The stability of any formulation may be assessed using the above protocol. In some embodiments, the stability of the formulation is assessed after storage of the formulation at an ambient temperature of 20-25 degrees Celsius for a period of at least one month, at least 6 months, at least one year, at least 1.5 years or at least two years. The results demonstrate that the formulations are stable under these conditions for at least one month, at least 6 months, at least one year, at least 1.5 years or at least two years.

### Example 26

### Release Profiles of Iloprost Formulations RDD/05/233, RDD/05/255 and RDD/05/257

Formulations comprising iloprost were prepared as follows.

Dispensing Iloprost for Formulation Preparation

A 5 ml volumetric flask and stainless steel spatula are accurately weighed. ˜300 mg iloprost is transferred into the volumetric flask using the spatula in a glove box. The flask and spatula are accurately weighed after adding iloprost. The amount of iloprost dispensed (W) is calculated. The iloprost on the spatula and in the flask is dissolved by adding ˜2 ml acetone. The spatula is washed by adding acetone drop wise into the flask and the solution is diluted to a desired volume and dispensed into vials such that each vial contains 6 mg iloprost. The sealed vials are stored under refigeration.

Formulation Preparation

To prepare formulations at TR153 and any surfactants, such as DPPC or DPPG-Na, are dissolved in acetone/water (75/25) (30 ml) in a container. If necessary the solution is war med to facilitate dissolution. 6 mg iloprost is added into the solution. The inner side of the vial is washed using a pipette to ensure all iloprost dissolves. The solution is mixed before spray drying.

Spray drying is performed on a customised Mini spray dryer (QDD specification) using the flow parameters listed in the table below. Powder is collected by a cyclone and recovered in a 15 ml glass pot. Process parameters can be converted with larger scale dryers.

The following formulations were prepared.

| | Formulation | | | | | |
|---|---|---|---|---|---|---|
| | 233 | 234 | 237 | 255 | 257 | 262 |
| Iloprost (mg) | 6 | 6 | 6 | 6 | 6 | 12 |
| DPPG-Na (mg) | 10 | 160 | 100 | 100 | 100 | 10 |
| Trehalose (mg) | 0 | 160 | 100 | 1740 | 0 | 0 |
| TR153 (mg) | 1984 | 1674 | 1740 | 100 | 1900 | 1978 |
| Acetone/water (75/25) | 30 | 80* | 80* | 80 | 80 | 30 |
| (mL) | | | | | | |
| Feed stock strength | 6.67 | 2.5 | 2.5 | 2.43 | 2.5 | 205 |
| (mg/mL) | | | | | | |
| Process conditions | | | | | | |
| Inlet temperature (° C.) | 70 | 70 | 70 | 70 | 70 | 70 |
| Corr outlet temperature | 50 | 50 | 50 | 50 | 50 | 50 |
| (° C.) | | | | | | |
| ATM pressure (bar) | 1.75 | 1.5 | 1.25 | 1.25 | 1.25 | 1.75 |
| Corr ATM flowrate | 18 | 17 | 16 | 16 | 16 | 18 |
| (L/min) | | | | | | |
| Dry air flowrate (bar) | 5 | 5 | 5 | 5 | 5 | 5 |
| Corr dry air pressure (L/sec) | 0.8 | 0.8 | 0.8 | 1 | 1 | 0.9 |
| Feed rate (%) | 90 | 90 | 90 | 90 | 90 | 90 |

*Acetone/water (80/20)

The rate of iloprost release was determined using a dissolution test performed as follows.

Dissolution Method for Determination of Release Rate of Iloprost from OED Particles

This method is designed for determining release rate of iloprost from discrete particles. Particles are well dispersed in the dissolution medium. This is to be facilitated by aerosolising the dry powder formulation into a dissolution vessel containing a known volume of the dissolution medium (PBS 7.4) using a PD-4 dry powder insufflator. The dissolution vessel comprises a vessel cap with an internal seal and contains dissolution medium and a stir bar on the bottom.

To measure the release rate a known volume (50 ml) of the dissolution medium is dispensed into the dissolution vessel. A stir bar is placed in the vessel. The vessel is sealed with a seal lined cap and left on a magnetic stirrer (200 rpm) in a 37° C. incubator until the temperature equilibrates. The vessel is removed from the incubator and the cap is replaced with one with a pre-drilled hole. The weight of the PD-4 dry powder insufflator is recorded. The end cap of the PD-4 dry powder insufflator is unscrewed and a dry powder formulation (10 mg) is placed into the dose chamber. The device is assembled and the weight is recorded. The mass of the formulation added is calculated. The device is attached to a compressed air supply through a connector. The outlet of the PD-4 dry powder insufflator is placed into the device by piercing the needle through the pre-drilled hole on the cap. The insufflator is actuated by switching on the compressed air at a pre-set actuation pressure of 1 bar for 0.5 seconds. The device is removed and the weight of the insufflator is recorded. The delivered powder mass is recorded by subtracting the weights before and after delivery.

The vessel is gently shaken and the cap is replaced. The vessel is gently shaken to ensure proper dispersion of powder in the dissolution medium. The time is recorded as TO. The vessel is left on the magnetic stirrer in a 37° C. incubator and later 1.5 ml solution is taken using a 2.5 ml syringe at each assigned time point. Iloprost is assayed after filtering through a 0.2 μm PTFE filter. To calculate the percentage of the iloprost release at each time point, the following equation is used:
%=Con×50 ml/Mass of the formulation delivered×% formulationx1000×100%

HPLC analysis was performed according to the protocol below.
Column: Spherisorb ODS 2, 3 μm, 125 mm×4.6 mm
Injection volume: 500 μl
Temperature: 20° C.
Flow rate: 1 ml/min
Detector: UV 200 nm
Mobile phase A: Acetonitrile/water (pH=2.0, phosphoric acid) (5/95, v/v)
Mobile phase B: Acetonitrile/water (pH=2.0, phosphoric acid) (95/5, v/v)

Gradient Steps:

| Step | Time | MP-A | MP-B | Curve |
|---|---|---|---|---|
| 0 | 0.5 | 50 | 50 | 0 |
| 1 | 5 | 50 | 50 | 0 |
| 2 | 1 | 10 | 90 | 1 |
| 3 | 1 | 10 | 90 | 0 |
| 4 | 1 | 50 | 50 | 1 |
| 5 | 0.3 | 50 | 50 | 0 |

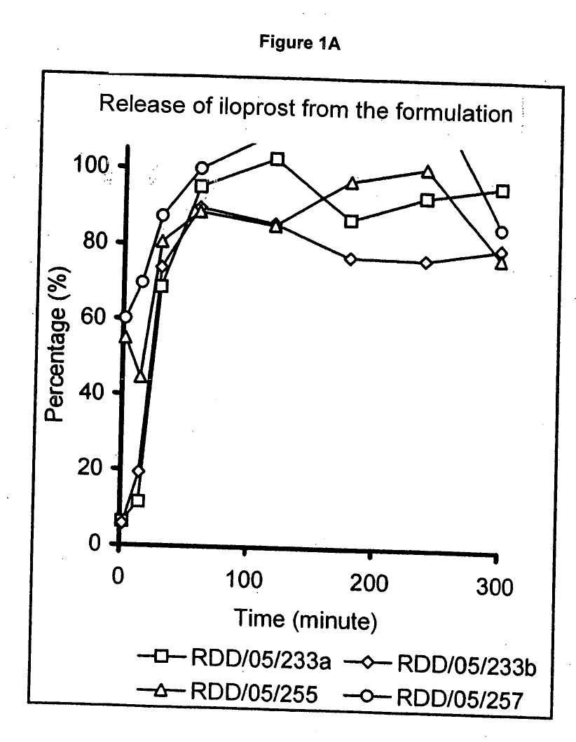

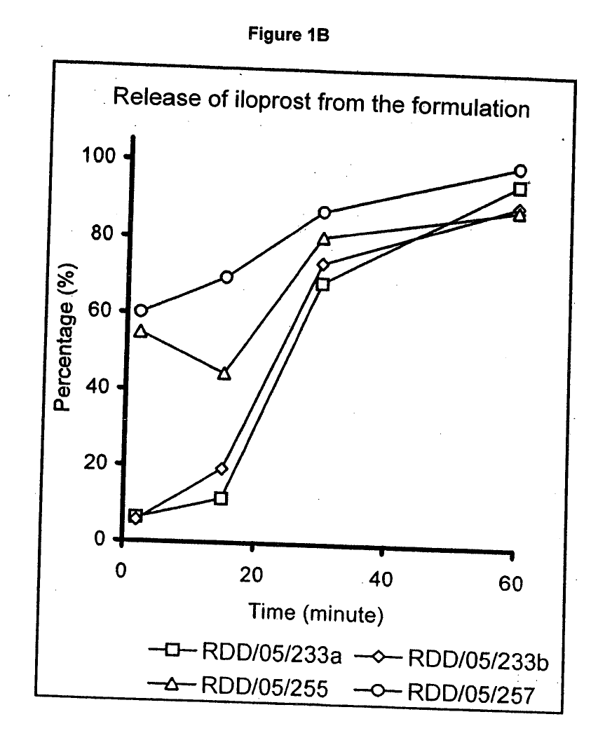

The release profiles for these formulations are indicated in FIG. 1A and FIG. 1B Formulations RDD/05/255 and 257 gave a high “burst” release. RDD/05/233 gave a low “burst” release, which demonstrated encapsulation of the iloprost within the

### Example 27

### Release Profiles of Iloprost Formulations RDD/05/233, RDD/05/234, RDD/05/237, RDD/05/255, RDD/05/257, RDD/05/262 RDD/05/267 RDD/05/270, RDD/05/273 and RDD/05/274

Formulations comprising iloprost were prepared as described in Example 26 above. The compositions of these formulations are described in the table below.

Formulation

| | RDD/05/ | | | | | | | | | |
|---|---|---|---|---|---|---|---|---|---|---|
| Formulation | 233 | 234 | 237 | 255 | 257 | 262 | 267 | 270 | 273 | 274 |
| Iloprost (mg) | 6 | 6 | 6 | 6 | 6 | 12 | 6 | 6 | 6 | 6 |
| DPPG-Na (mg) | 10 | 160 | 100 | 100 | 100 | 10 | 2.5 | 0 | 2.5 | 50 |
| DPPC (mg) | 0 | 0 | 0 | 0 | 0 | 0 | 0 | 20 | 20 | 20 |
| Trehalose (mg) | 0 | 160 | 100 | 1740 | 0 | 0 | 0 | 0 | 0 | 0 |
| TR153 (mg) | 1984 | 1674 | 1740 | 100 | 1900 | 1978 | 1992 | 1974 | 1972 | 1924 |
| Acetone/water | 30 | 80* | 80* | 80 | 80 | 30 | 30 | 30 | 30 | 30 |
| (75/25) (mL) | | | | | | | | | | |
| Feed stock strength | 6.67 | 2.5 | 2.5 | 2.43 | 2.5 | 2.5 | 6.67 | 6.67 | 6.67 | 6.67 |
| (mg/mL) | | | | | | | | | | |

*Acetone/water (80/20)

The rate of iloprost release was determined using a dissolution test performed as described in Example 26 above. Released iloprost was measured as described in Example 26.

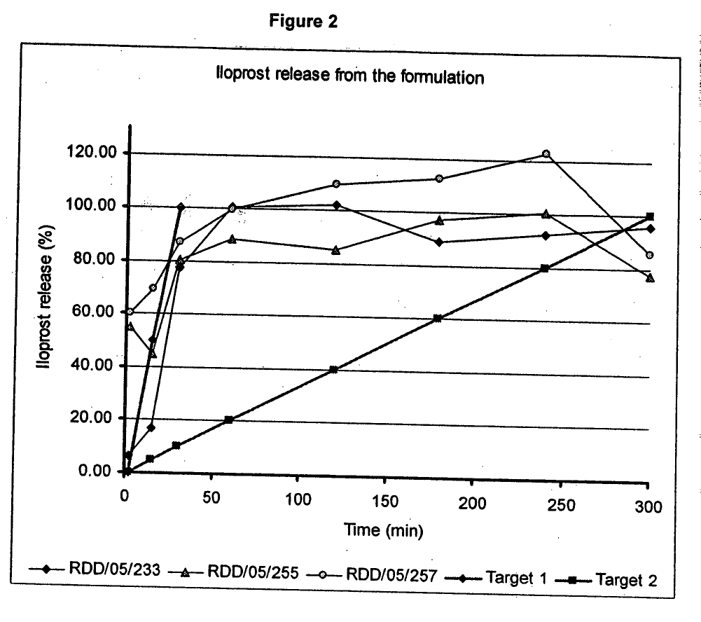

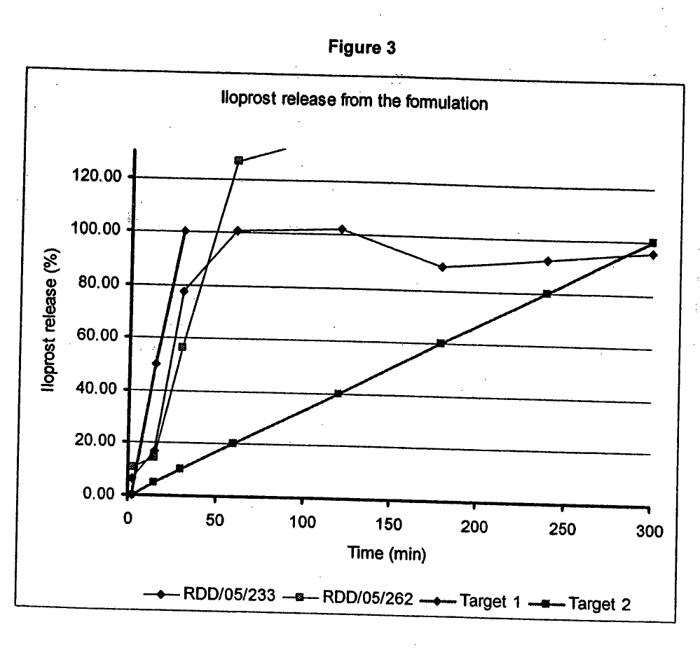

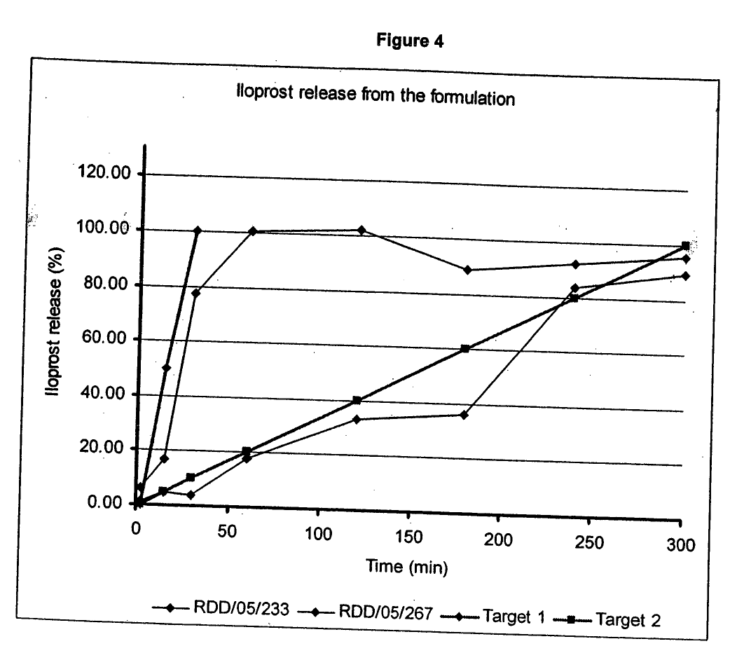

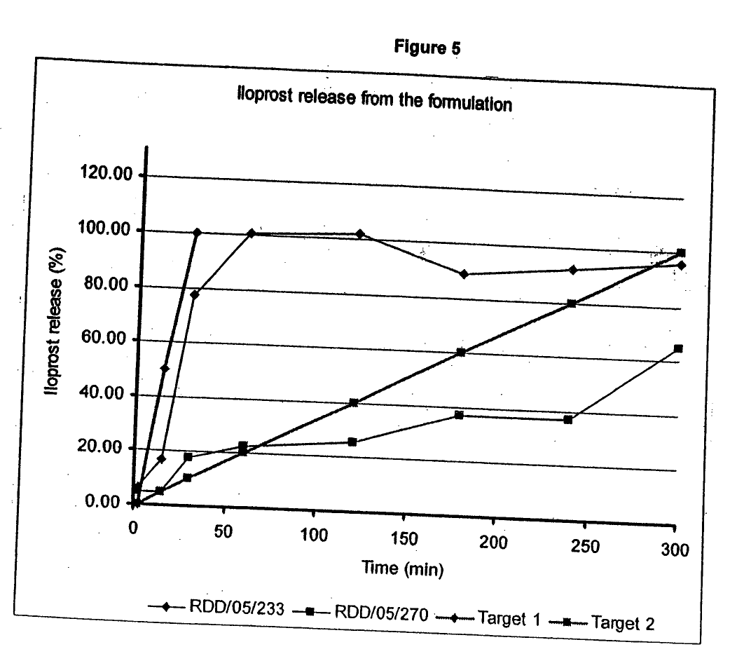

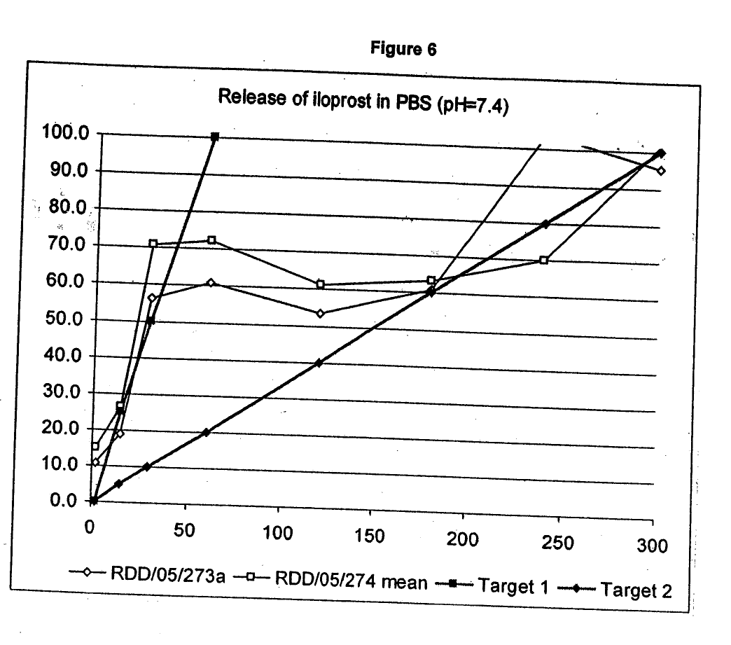

The release profiles for these formulations are indicated in FIGS. 2-6.

As shown in FIG. 2, reducing the stock solution total solid concentration increases the “burst release (CompareRDD/05/257 with RDD/05/233). Increasing iloprost loading from 0.3% to 0.6% did not increase either the “burst” release or the overall release rate (RDD/05/233 and RDD/05/262). Reducing DPPG-Na content reduces the release rate. Replacing DPPG-Na with the more lipophilic lecithin DPPC reduces the release rate. The DPPC formulation dispersed well in the dissolution medium and did not give a high “burst” release even when the weight ratio was doubled compared to DPPG-Na (RDD/05/233 and RDD/05/270). DPPC improves wettability of the formulations. The formulation shows an enhanced release rate. Increasing the DPPG-Na content from 2.5 mg to 50 mg (in 2 g formulation) shows a marginal increase in release rate (RDD/05/273 and RDD/05/274).

### Example 28

### In Vivo Assessment of Formulations Comprising Iloprost and/or Another Pharmaceutical Agent to be Administered in Addition to Iloprost

Formulations containing iloprost and/or another pharmaceutical agent to be administered in addition to iloprost can be assessed in vivo as follows. An amount of microparticles (approximately 1-100 mg) is loaded into a Penn Century Dry Powder Delivery Device. The device is then used to administer the dose intratracheally in a single inhalation to a tracheotomized dog. Blood samples are then drawn at specified time points (e.g., 5, 15, 30, 60, 120, 240, 360, 480 min) and plasma is prepared and processed to enrich for iloprost and remove interfering substances. The processed samples are then analyzed for iloprost concentration using a liquid chromatography tandem mass spectrometry method. Standard pharmacokinetic analysis is then performed on the plasma drug concentration time curves to derive common pharmacokinetic parameters such as bioavailability and plasma drug half-life.

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention, which is delineated by the appended claims.

All of the references cited herein are incorporated in their entirety by reference thereto.

What is claimed is:

1. A composition comprising a solid dose delivery system comprising a vehicle and an effective amount of iloprost wherein the vehicle comprises a hydrophobic derivatized carbohydrate (HDC).

2. The composition according to claim 1, further comprising at least one physiologically acceptable glass selected from the group consisting of carboxylate, nitrate, sulfate, and bisulfate.

3. The composition according to claim 1, wherein the HDC has a carbohydrate backbone and more than one hydroxyl group substituted with a less hydrophilic derivative thereof.

4. The composition according to claim 3, wherein the derivative is an ester or ether of any carbon chain length or type or any functional modifications thereof, wherein the functional modifications are selected from the group consisting of replacing the oxygen atom by a heteroatom.

5. The composition according to claim 4, wherein the HDC is selected from the group consisting of 6:6′-bis(β-Tetraacetyl glucuronyl)hexaacetyl trehalose, sorbitol hexaacetate, α-Glucose pentaacetate, β-Glucose pentaacetate, 1-0-Octyl-β-D-Glucose tetraacetate, trehalose octaacetate, trehalose octapropanoate, sucrose octaacetate, sucrose octapropanoate, cellobiose octaacetate, cellobiose octapropanoate, raffinose undecaacetate and raffinose undecapropanoate.

6. The composition according to claim 1, wherein the guest substance has increased stability in the presence of elevated temperatures or organic solvents.

7. The composition according to claim 1, wherein the form of the solid dose is selected from the group consisting of microparticles, microspheres and powders.

8. The composition according to claim 1 further comprising a pharmaceutical agent in addition to iloprost, wherein said pharmaceutical agent in addition to iloprost is selected from the group consisting of vasodilators, antihypertensive agents, cardiovascular drugs, an endothelin receptor antagonist, a PDE inhibitor, and a calcium channel blocker, wherein the iloprost and the at least one additional agent are provided at dosages sufficient to ameliorate at least one symptom associated with PH.

9. The composition of claim 1, wherein the vehicle comprises a hydrophobic derivatized carbohydrate (HDC) in which the iloprost can be dried and stored.

10. The composition of claim 1, wherein the vehicle comprises a hydrophobic derivatized carbohydrate (HDC) in which the iloprost can be dried and stored without losses in activity.

11. The composition of claim 1, wherein the hydrophobic derivatized carbohydrate (HDC) is non-toxic.

12. The composition of claim 1, wherein the vehicle comprises a hydrophobic derivatized carbohydrate (HDC) which is glassy or amorphous.

13. The composition of claim 1, wherein composition is capable of controlled release of the iloprost.

14. The composition of claim 1, wherein the composition is resistant to devitrification.

15. The composition of claim 1, wherein the HDC is a carbohydrate no greater than a pentasaccharide, and wherein more than one hydroxyl group of the HDC is derivatized as an ester or ether.

16. The composition of claim 1, further comprising a stabilizing polyol.

17. The composition according to claim 16, further comprising at least one physiologically acceptable glass selected from the group consisting of carboxylate, nitrate, sulfate, and bisulfate.

18. The composition according to claim 16, wherein the HDC has a carbohydrate backbone and more than one hydroxyl group substituted with a less hydrophilic derivative thereof.

19. The composition according to claim 18, wherein the HDC is selected from the group consisting of 6:6′-bis(β-Tetraacetyl glucuronyl)hexaacetyl trehalose, sorbitol hexaacetate, α-Glucose pentaacetate, β-Glucose pentaacetate, 1-0-Octyl-β-D-Glucose tetraacetate, trehalose octaacetate, trehalose octapropanoate, sucrose octaacetate, sucrose octapropanoate, cellobiose octaacetate, cellobiose octapropanoate, raffinose undecaacetate and raffinose undecapropanoate.

20. The composition according to claim 16, wherein the iloprost has increased stability in the presence of elevated temperatures or organic solvents.

21. The composition according to claim 16, wherein the form of the solid dose is selected from the group consisting of microparticles, microspheres, and powders.

22. The composition of claim 1, further comprising a surfactant.

23. The composition of claim 22, wherein said surfactant has a hydrophile-lipophile balance of at least about 3.

24. The composition of claim 23, wherein said surfactant is selected from the group consisting of dipalmitoyl phosphatidylglycerol, dipalmitoyl phosphatidylcholine, glyceryl monostearate, sorbitan monolaurate, polyoxyethylene-4-lauryl ether, polyethylene glycol 400 monostearate, polyoxyethylene-4-sorbitan monolaurate, polyoxyethylene-20-sorbitan monopalmitate, polyoxyethylene-40-stearate, sodium oleate and sodium lauryl sulfate and lung surfactants.

25. The composition of claim 23, wherein the composition provides increased bioavailability of the iloprost.

26. The composition according to claim 23, wherein the composition is obtained by dissolving or suspending the iloprost, the surface active agent and the hydrophobically derivatized carbohydrate in at least one solvent therefor and evaporating the solvent from the mixture.

27. The composition according to claim 23, wherein the evaporating is by spray drying.

28. The composition according to claim 23 further comprising a pharmaceutical agent in addition to iloprost, wherein said pharmaceutical agent in addition to iloprost is selected from the group consisting of vasodilators, antihypertensive agents, cardiovascular drugs, an endothelin receptor antagonist, a PDE inhibitor, and a calcium channel blocker, wherein the iloprost and the at least one additional agent are provided at dosages sufficient to ameliorate at least one symptom associated with PH.

29. The composition according to claim 23, wherein the hydrophobically derivatized carbohydrate is selected from the group consisting of sorbitol hexaacetate (SHAC), α-glucose pentaacetate (α-GPAC), β-glucose pentaacetate (β-GPAC), 1-0-Octyl-β-D-glucose tetraacetate (OGTA), trehalose octaacetate (TOAC), trehalose octapropionate (TOP), trehalose octa-3,3,dimethylbutyrate (T033DMB), trehalose diisobutyrate hexaacetate, trehalose octaisobutyrate, lactose octaacetate, sucrose octaacetate (SOAC), cellobiose octaacetate (COAC), raffinose undecaacetate (RUDA), sucrose octapropanoate, cellobiose octapropanoate, raffinose undecapropanoate, tetra-0-methyl trehalose, trehalose octapivalate, trehalose hexaacetate dipivalate and di-0-methyl-hexa-0-actyl sucrose and mixtures thereof.

30. The composition according to claim 23, wherein the hydrophobically derivatized carbohydrate is a trehalose derivative and comprises: 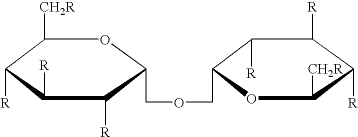
where R represents a hydroxyl group, or less hydrophilic derivative thereof, including an ester or ether or any functional modifications thereof where at least one R is not hydroxyl but a hydrophobic derivative; where functional modifications include where the oxygen atom is replaced by a heteroatom, such as N or S and where R can be of any chain length from C₂ upwards and can be straight, branched, cyclic or modified and mixtures thereof.

31. A composition according to claim 23, comprising the pharmaceutical composition in powder form, suspended in an aqueous solution.

32. The composition of claim 16, wherein said stabilizing polyol is selected from the group consisting of monosaccharides, disaccharides, trisaccharides, oligosaccharides and their corresponding sugar alcohols, polysaccharides and chemically modified carbohydrates such as hydroxyethyl starch and sugar copolymers and Ficoll.

33. The composition of claim 16, wherein said stabilizing polyol is trehalose.

34. The composition of claim 1, wherein said HDC is 6:6′-bis(β-Tetraacetyl glucuronyl)hexaacetyl trehalose.

35. A composition comprising iloprost and a modified glycoside, said modified glycoside having the formula:
(Y)ₙ—X
wherein Y represents a saccharide subunit, n is 1-6, and, when n is greater than 1, the subunits are linked in a linear or branch chain by glycosidic linkages; and
wherein X is a 5 or 6 carbon monosaccharide polyalcohol, and wherein the polyalcohol has a hydroxy group linked via a glycosidic bond to the anomeric carbon of one of the saccharide subunits; and
wherein the glycoside has at least one hydroxy group derivatized in the form of an ester, mixed ester, ether or mixed ether; and
wherein the modified glycoside is in the form of a vitreous glass matrix and has a bioactive substance incorporated therein.

36. The composition of claim 35 wherein the saccharide subunits, Y, are the same or different and are selected from the group consisting of glucose, galactose, fructose, ribulose, mannose, ribose, arabinose, xylose, lyxose, allose, altrose, and gulose.

37. The composition of claim 35 wherein the polyalcohol is selected from the group consisting of erythritol, ribitol, xylitol, galactitol, glucitol and mannitol.

38. The composition of claim 35 wherein the modified glycoside is a hydrogenated maltooligosaccharide or isomaltooligosaccharide.

39. The composition of claim 38, wherein the hydrogenated maltooligosaccharide is selected from the group consisting of maltotritol, maltotetraitol, maltopentaitol, maltohexaitol, maltooctaitol, maltononaitol and maltodecaitol.

40. The composition of claim 35 wherein the modified glycoside is selected from the group consisting of hydrophobic esters, mixed esters, ethers or mixed ethers of a glycoside of a sugar alcohol.

41. The composition of claim 35 wherein said modified glycoside is selected from the group consisting of lactitol nonaacetate, palatinit nonaacetate, glycopyranosyl sorbitol nonaacetate, glucopyranosyl mannitol nonaacetate, maltitol nonaacetate and mixtures thereof.

42. The composition according to claim 35, further comprising at least one physiologically acceptable glass selected from the group consisting of carboxylate, nitrate, sulfate, bisulfate, a hydrophobic carbohydrate derivative, and combinations thereof.

43. The composition according to claim 35, wherein the composition is in the form of a solid delivery system selected from the group consisting of microparticles, microspheres, and powders.

44. The composition of claim 35, further comprising a surfactant.

45. The composition of claim 44, wherein said surfactant has a hydrophile-lipophile balance of at least about 3.

46. The composition of claim 45, wherein said surfactant is selected from the group consisting of dipalmitoyl phosphatidylglycerol, dipalmitoyl phosphatidylcholine, glyceryl monostearate, sorbitan monolaurate, polyoxyethylene-4-lauryl ether, polyethylene glycol 400 monostearate, polyoxyethylene-4-sorbitan monolaurate, polyoxyethylene-20-sorbitan monopalmitate, polyoxyethylene-40-stearate, sodium oleate and sodium lauryl sulfate and lung surfactants.

47. The composition of claim 35, wherein further comprising a stabilizing polyol.

48. The composition of claim 47, wherein said stabilizing polyol is selected from the group consisting of monosaccharides, disaccharides, trisaccharides, oligosaccharides and their corresponding sugar alcohols, polysaccharides and chemically modified carbohydrates such as hydroxyethyl starch and sugar copolymers and Ficoll.

49. The composition of claim 48, wherein said stabilizing polyol is trehalose.

50. The composition according to claim 35 further comprising a pharmaceutical agent in addition to iloprost, wherein said pharmaceutical agent in addition to iloprost is selected from the group consisting of vasodilators, antihypertensive agents, cardiovascular drugs, an endothelin receptor antagonist, a PDE inhibitor, and a calcium channel blocker, wherein the iloprost and the at least one additional agent are provided at dosages sufficient to ameliorate at least one symptom associated with PH.

51. A pharmaceutical composition for pulmonary delivery comprising:
an intimate mixture of a therapeutically effective amount of iloprost, a surface active agent, and a hydrophobically derivatized carbohydrate (HDC) where the composition is in powder form.

52. The composition of claim 51, wherein said composition provides increased bioavailability of the iloprost to the pulmonary system.

53. The composition of claim 51, wherein the surface active agent forms a continuous phase with the HDC.

54. The composition according to claim 51, wherein the surface active agent is a surfactant with a hydrophile-lipophile balance.

55. The composition according to claim 54, wherein the hydrophile-lipophile balance is of at least about 3.

56. The composition according to claim 55, wherein the surfactant is selected from the group consisting of dipalmitoyl phosphatidylglycerol, dipalmitoyl phosphatidylcholine, glyceryl monostearate, sorbitan monolaurate, polyoxyethylene-4-lauryl ether, polyethylene glycol 400 monostearate, polyoxyethylene-4-sorbitan monolaurate, polyoxyethylene-20-sorbitan monopalmitate, polyoxyethylene-40-stearate, sodium oleate sodium lauryl sulfate and lung surfactants.

57. The composition according to claim 51, wherein mucosal delivery is via by-inhalation delivery.

58. The composition according to claim 51, wherein the powder contains particles with a mass median aerodynamic diameter of about 0.1 to 10 microns.

59. The composition according to claim 51, wherein the powder contains particles with a mass median aerodynamic diameter of about 0.5 to 5 microns.

60. The composition according to claim 51, wherein the powder contains particles with a mass median aerodynamic diameter of about 1 to 4 microns.

61. The composition according to claim 51, wherein the intimate mixture is obtained by dissolving or suspending the bioactive agent and the hydrophobically derivatized carbohydrate in at least one solvent therefor and evaporating the solvent from the mixture.

62. The composition according to claim 51, wherein the evaporating is by spray drying.

63. The composition according to claim 51 further comprising a pharmaceutical agent in addition to iloprost, wherein said pharmaceutical agent in addition to iloprost is selected from the group consisting of vasodilators, antihypertensive agents, cardiovascular drugs, an endothelin receptor antagonist, a PDE inhibitor, and a calcium channel blocker, wherein the iloprost and the at least one additional agent are provided at dosages sufficient to ameliorate at least one symptom associated with PH.

64. The composition according to claim 51, wherein the hydrophobically derivatized carbohydrate is selected from the group consisting of sorbitol hexaacetate (SHAC), α-glucose pentaacetate (α-GPAC), β-glucose pentaacetate (β-GPAC), 1-0-Octyl-β-D-glucose tetraacetate (OGTA), trehalose octaacetate (TOAQ, tetralose octapropionate (TOP), trehalose octa-3,3,dimethylbutyrate (TO33DMB), trehalose diisobutyrate hexaacetate, trehalose octaisobutyrate, lactose octaacetate, sucrose octaacetate (SOAC), cellobiose octaacetate (COAC), raffinose undecaacetate (RUDA), sucrose octapropanoate, cellobiose octapropanoate, raffinose undecapropanoate, tetra-0-methyl trehalose, trehalose octapivalate, trehalose hexaacetate dipivalate and di-0-methyl-hexa-0-actyl sucrose and mixtures thereof.

65. The composition according to claim 51, wherein the hydrophobically derivatized carbohydrate is a trehalose derivative and comprises: 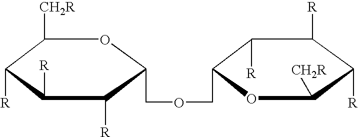
where R represents a hydroxyl group, or less hydrophilic derivative thereof, including an ester or ether or any functional modifications thereof where at least one R is not hydroxyl but a hydrophobic derivative; where functional modifications include where the oxygen atom is replaced by a heteroatom, such as N or S and where R can be of any chain length from C₂ upwards and can be straight, branched, cyclic or modified and mixtures thereof.

66. A composition comprising the powders of claim 51 suspended in an aqueous solution.

67. The composition according to claim 51, wherein the powder contains particles with a mass median aerodynamic diameter of 1.5-3 microns.

68. The composition of claim 51, wherein said HDC is 6:6′-bis(β-Tetraacetyl glucuronyl)hexaacetyl trehalose.

69. A composition comprising iloprost and 6:6′-bis(β-Tetraacetyl glucuronyl)hexaacetyl trehalose.

70. The composition of claim 69, further comprising a surfactant.

71. The composition of claim 70 further comprising trehalose.

72. The composition of claim 69 wherein said iloprost is present at a concentration from about 0.01%-about 30% by weight.

73. The composition of claim 69 wherein said iloprost is present at a concentration from about 0.05%-about 20% by weight.

74. The composition of claim 69 wherein said iloprost is present at a concentration from about 0.1%-about 5% by weight.

75. The composition of claim 70, wherein said surfactant is selected from the group consisting of dipalmitoyl phosphatidylglycerol and dipalmitoyl phosphatidylcholine.

76. The composition of claim 70, wherein said surfactant is present in a concentration of about 0.01%-about 30% by weight.

77. The composition of claim 70, wherein said surfactant is present in a concentration of 0.1%-20% by weight.

78. The composition of claim 70, wherein said surfactant is present in a concentration of about 0.1%-about 10% by weight.

79. The composition of claim 70, wherein said surfactant is present in a concentration of about 0.1%-5% by weight.

80. Microparticles comprising iloprost therein.

81. The microparticles of claim 80, wherein said microparticles provide a dosage of iloprost which provides an efficacious amount of iloprost when said microparticles are administered 1 to 10 times daily.

82. The microparticles of claim 80, wherein said microparticles provide a dosage of iloprost which provides an efficacious amount of iloprost when said microparticles are administered 1 to 4 times daily.

83. The microparticles of claim 80, wherein said microparticles provide a dosage of iloprost which provides an efficacious amount of iloprost when said microparticles are administered 3 to 4 times daily.

84. The microparticles of claim 80, wherein said microparticles are in the form of a dry powder.

85. The microparticles of claim 80, wherein said microparticles release an effective amount of iloprost over a duration of at least two hours from inhalation of said microparticles by a human subject.

86. The microparticles of claim 80, wherein substantially all of the iloprost is released by 24 hours from inhalation of said microparticles by a human subject.

87. The microparticles of claim 80 further comprising a carbohydrate or derivative of a carbohydrate.

88. The microparticles of claim 87, wherein said derivative of a carbohydrate is an ether or ester.

89. The microparticles of claim 88, wherein said derivative of a carbohydrate is an ester.

90. A method of treating PH, comprising administering effective amounts of microparticles comprising iloprost to an individual suffering from PH.

91. An inhalation device comprising microparticles comprising iloprost.

92. The device of claim 91, wherein said device is selected from the group consisting of a dry powder inhalation device and a metered dose inhaler.
